(19) United States
(12) Patent Application Publication
(10) Pub. No.: US 2014/0017308 A1
(43) Pub. Date: Jan. 16, 2014

(54) COMPOSITIONS COMPRISING A FATTY ACID OIL MIXTURE, A FREE FATTY ACID, AND A STATIN

(71) Applicants: Svein Olaf Hustvedt, Oslo (NO); Gunnar Berge, Oslo (NO); Preben Houlberg Olesen, Copenhagen NV (DK); Anette Müllertz, Chartottenlund (DK)

(72) Inventors: Svein Olaf Hustvedt, Oslo (NO); Gunnar Berge, Oslo (NO); Preben Houlberg Olesen, Copenhagen NV (DK); Anette Müllertz, Chartottenlund (DK)

(73) Assignee: Pronova Biopharma Norge AS, Lysaker (NO)

(21) Appl. No.: 13/825,765

(22) PCT Filed: Sep. 8, 2011
(86) PCT No.: PCT/IB11/02755

§ 371 Date: Oct. 3, 2013

Related U.S. Application Data
(60) Provisional application No. 61/381,061, filed on Sep. 8, 2010.

(51) Int. Cl. A61K 31/40; A61K 45/06; A61K 31/202

(52) U.S. Cl. CPC A61K 31/40; A61K 31/202; A61K 45/06
USPC 424/456; 424/493; 514/423; 514/560

(57) ABSTRACT

Compositions comprising a fatty acid oil mixture, at least one free fatty acid, and at least one statin or pharmaceutically acceptable salt, hydrate, solvate, or complex thereof, and uses thereof are disclosed. Further disclosed are preconcentrates capable of forming a self-nanoemulsifying drug delivery system (SNEDDS), a self-microemulsifying drug delivery system (SMEDDS), or self-emulsifying drug delivery systems (SEDDS) in an aqueous solution.

This application claims priority to U.S. Provisional Application No. 61/381,061, filed on Sep. 8, 2010, which is incorporated herein by reference in its entirety.

The present disclosure relates generally to compositions comprising a fatty acid oil mixture, at least one free fatty acid, and at least one statin or pharmaceutically acceptable salt, hydrate, solvate, or complex thereof, and methods of use thereof. The fatty acid oil mixture may comprise omega-3 fatty acids, such as eicosapentaenoic acid (EPA) and docosahexaenoic acid (DHA) in ethyl ester or triglyceride form. Further disclosed are preconcentrate compositions and self-nanoemulsifying drug delivery systems (SNEDDS), self-microemulsifying drug delivery systems (SMEDDS) and self-emulsifying drug delivery systems (SEDDS).

The compositions presently disclosed may be administered, e.g., in capsule, caplet, or tablet form, to a subject for therapeutic treatment and/or regulation of at least one health problem including, for example, irregular plasma lipid levels, cardiovascular functions, immune functions, visual functions, insulin action, neuronal development, hypertriglyceridemia, hypercholesterolemia, mixed dyslipidemia, heart failure, and post myocardial infarction (MI). The present disclosure further relates to a method of increasing hydrolysis, solubility, bioavailability, absorption, and/or any combination thereof.

In humans, cholesterol and triglycerides are part of lipoprotein complexes in the bloodstream and can be separated via ultracentrifugation into high-density lipoprotein (HDL), intermediate-density lipoprotein (IDL), low-density lipoprotein (LDL), and very-low-density lipoprotein (VLDL) fractions. Cholesterol and triglycerides are synthesized in the liver, incorporated into VLDL, and released into the plasma. Conditions characterized by abnormally high blood cholesterol and/or lipid values include hypercholesterolemia, hyperlipidemia (hyperlipoproteinemia), hypertriglyceridemia, and mixed dyslipidemia. High levels of total cholesterol (total-C), LDL-C, and apolipoprotein B (a membrane complex for LDL-C and VLDL-C) may promote human atherosclerosis. Decreased levels of HDL-C and its transport complex, apolipoprotein A, are also associated with the development of atherosclerosis. Cardiovascular morbidity and mortality in humans can vary directly with the level of total-C and LDL-C, and inversely with the level of HDL-C. In addition, research suggests that non-HDL cholesterol is an indicator of hypertriglyceridemia, vascular disease, atherosclerotic disease, and related conditions. In fact, the NCEP ATP III (National Cholesterol Education Program Adult Treatment Panel HO report specifies non-HDL cholesterol reduction as a treatment objective.

Omega-3 fatty acids may regulate plasma lipid levels, cardiovascular and immune functions, insulin action, and neuronal development, and visual function. Marine oils, also commonly referred to as fish oils, are a source of omega-3 fatty acids, including eicosapentaenoic acid (EPA) and docosahexaenoic acid (DHA), which have been found to regulate lipid metabolism. Plant-based oils and microbial oils are also sources of omega-3 fatty acids. Omega-3 fatty acids may have beneficial effects on the risk factors for cardiovascular diseases, for example hypertension and hypertriglyceridemia, and on the coagulation factor VII phospholipid complex activity. Omega-3 fatty acids may also lower serum triglycerides, increase serum HDL cholesterol, lower systolic and diastolic blood pressure and/or pulse rate, and may lower the activity of the blood coagulation factor VII-phospholipid complex. Further, omega-3 fatty acids are generally well-tolerated, without giving rise to severe side effects.

Several formulations omega-3 fatty acids have been developed. For example, one form of omega-3 fatty acid oil mixture is a concentrate of primary omega-3, long chain, polyunsaturated fatty acids from fish oil containing DHA and EPA, such as sold under the trademark Omacor®/Lovaza™/Zodin®/Seacor®. See, e.g., U.S. Pat. Nos. 5,502,077, 5,656,667 and 5,698,594. In particular, each 1000 mg capsule of Lovaza™ contains at least 90% omega-3 ethyl ester fatty acids (84% EPA/DHA); approximately 465 mg EPA ethyl ester and approximately 375 mg DHA ethyl ester.

However, evidence suggests that long chain fatty acids and alcohols of up to at least C₂₄ are reversibly interconverted. Enzyme systems exist in the liver, fibroblasts, and the brain that convert fatty alcohols to fatty acids. In some tissues, fatty acids can be reduced back to alcohols. The carboxylic acid functional group of fatty acid molecules targets binding, but this ionizable group may hinder the molecule from crossing the cell membranes, such as of the intestinal wall. As a result, carboxylic acid functional groups are often protected as esters. The ester is less polar than the carboxylic acid, and may more easily cross the fatty cell membranes. Once in the bloodstream, the ester can be hydrolyzed back to the free carboxylic acid by enzyme esterase in the blood. It may be possible that the plasma enzymes do not hydrolyze the ester fast enough, however, and that the conversion of ester to free carboxylic acid predominantly takes place in the liver. Ethyl esters of polyunsaturated fatty can also be hydrolyzed to free carboxylic acids in vivo.

The biosynthesis of cholesterol by human liver cells is a multistep process starting with acetyl-CoA. In the early part of this process, hydroxymethyl-glutaryl-CoA (HMG-CoA) is reduced forming R-mevalonic acid. This process is catalyzed by the enzyme HMG-CoA reductase. Several compounds inhibit this enzyme and thereby inhibit the biosynthesis of cholesterol (see FIG. 1). These inhibitors are named statins or HMG-CoA reductase inhibitors, and are frequently used as drugs for reduction of plasma cholesterol. Examples of statins include atorvastatin, cerivastatin, fluvastatin, itavastatin, lovastatin, mevastatin, rosuvastatin, simvastatin, pravastatin, and pitavastatin. The chemical formulae for various statins are shown in FIG. 2.

Atorvastatin and atorvastatin-like drugs, and processes of preparation, compositions, and uses thereof are described, for example, in U.S. Pat. Nos. 4,681,893, 5,969,156, 6,262,092, 6,486,182, 6,528,660, 6,600,051, 6,605,636, 6,605,727, 6,613,916, 6,646,133, 6,730,797, 6,737,430, 6,750,353, 6,835,742, 6,867,306, 6,891,047, 6,992,194, 7,030,151, 7,074,818, 7,074,940, 7,112,604, 7,122,681, 7,129,265, 7,144,916, 7,151,183, 7,161,012, 7,186,848, 7,189,861, 7,193,090, 7,256,212, 7,342,120, 7,361,772, 7,411,075, 7,414,141, 7,429,613, 7,456,297, 7,468,444, 7,488,750, 7,501,450, 7,538,136, 7,615,647, 7,645,888, 7,655,692, 7,674,923, 7,732,623, 7,745,480 and U.S. Pat. No. 7,772,273.

Simvastatin and simvastatin-like drugs, and processes of preparation, compositions, and uses thereof are described, for example, in U.S. Pat. Nos. 4,444,784; 5,393,893, 5,763,646, 5,763,653, 6,100,407, 6,252,091, 6,271,398, 6,307,066, 6,331,641, 6,384,238, 6,506,929, 6,521,762, 6,541,511, 6,573,385, 6,573,392, 6,576,775, 6,603,022, 6,686,481, 6,696,086, 6,797,831, 6,825,362, 6,833,461, 6,984,399, 6,995,277, 7,205,415, 7,528,265, 7,678,928 and U.S. Pat. No. 7,700,339.

Pravastatin and pravastatin-like drugs, and processes of preparation, composition, and uses thereof, are described, for example, in U.S. Pat. Nos. 4,346,227; 4,857,522, 5,047,549, 5,140,012, 5,155,229, 5,180,589, 5,260,305; 5,180,589, 5,260,305, 5,942,423, 6,204,032, 6,274,360, 6,306,629, 6,566,120, 6,682,913, 6,696,599, 6,716,615, 6,740,775, 6,750,366, 6,790,984, 6,905,851, 6,936,731, 6,967,218, 7,001,019, 7,056,710, 7,078,558, 7,189,558, 7,223,590, 7,262,218, 7,425,644, 7,582,464 and U.S. Pat. No. 7,642,286.

Fluvastatin and fluvastatin-like drugs, and processes of preparation, composition, and uses thereof, are described, for example, in U.S. Pat. Nos. 6,858,643, 7,241,800, 7,368,468, 7,368,581, 7,414,140, 7,432,380, 7,662,848 and U.S. Pat. No. 7,687,642.

Lovastatin and lovastatin-like drugs, and processes of preparation, composition, and uses thereof, are described, for example, in U.S. Pat. Nos. 4,866,186, 5,082,650, 5,409,820, 5,595,734, 5,712,130, 5,763,646, 6,197,560, 6,472,542, 6,500,651, 6,521,762, 6,696,086, 6,984,399, 7,052,886 and U.S. Pat. No. 7,566,792.

Rosuvastatin and rosuvastatin-like drugs, and processes of preparation, composition, and uses thereof, are described, for example, in U.S. Pat. Nos. 6,858,618, 7,161,004, 7,179,916, 7,244,844, 7,396,927, 7,511,140, 7,566,782, 7,582,759, 7,612,203, 7,692,008, 7,692,009, 7,672,010, 7,741,482 and U.S. Pat. No. 7,777,034.

Cerivastatin and cerivastatin-like drugs, and processes of preparation, composition, and uses thereof, are described, for example, in U.S. Pat. No. 6,511,985.

Itavastatin and itavastatin-like drugs, and processes of preparation, composition, and uses thereof, are described, for example, in Saito et al., Atherosclerosis, 151:1, 154 (July 2000); Teramoto et al., Atherosclerosis, 151:1, 53 (July 2000); Kithhara et al., Atherosclerosis, 151:1, 295 (2000), and further publications in the same issue.

Mevastatin and meavastatin-like drugs, and processes of preparation, composition, and uses thereof, are described, for example, in U.S. Pat. Nos. 6,384,238, 6,531,507, 6,583,295, 6,695,969, 6,806,290, 6,838,566, 7,078,558, 7,141,602 and U.S. Pat. No. 7,582,464.

Pitavastatin and pitavastatin-like drugs, and processes of preparation, composition, and uses thereof, are described, for example, in U.S. Pat. Nos. 6,777,552, 7,238,826, 7,241,800, 7,301,046, 7,459,447, 5,598,233 and U.S. Pat. No. 7,776,881.

Statins may be used in the form of salts; specific examples include calcium salts of atorvastatin, itavastatin, rosuvastatin, and pitavastatin: and sodium salts of pravastatin and fluvastatin. Statins may also be in lactone form, such as simvastatin, mevastatin, and lovastatin. Further, statins may exist in various crystalline forms and/or in amorphous form. For example, atorvastatin calcium salt can exist in an amorphous form or in different crystalline forms. See, e.g., WO 97/3958, WO 97/3959, WO 01/36384, WO 02/41834, WO 02/43732, WO 02/51804, and WO 02/57229. Processes for the preparation of amorphous atorvastatin calcium are described, for example, in WO 97/3960, WO 00/71116, WO 01/28999, WO 01/42209, WO 02/57228, and WO 02/59087.

The oral bioavailability of statins is generally low: atorvastatin (20%), simvastatin (less than 5%), pravastatin (18%) and rosuvastatin (20%). Active drug substances in an amorphous form may be better soluble and dissolve more rapidly than in a crystalline form. Atorvastatin calcium in amorphous form is claimed to have higher bioavailability than crystalline forms of the same salt.

The half-life of statins may vary over a wide range, e.g., pravastatin (about 0.8 hours), simvastatin (about 2-3 hours), atorvastatin (about 20 hours) and rosuvastatin (about 20 hours). The daily clinical dose of various statins may also vary, e.g., atorvastatin (10-80 mg), cerivastatin (0.2-0.3 mg), fluvastatin (20-80 mg), lovastatin (20-80 mg), pravastatin (10-40 mg), and simvastatin (5-80 mg).

Further, statins may be unstable. For example, atorvastatin calcium is susceptible to heat, light, oxygen, moisture, and low pH. At low pH, atorvastatin calcium is converted from the carboxylic acid form to the lactone form, and in presence of oxygen various oxidation products are formed. Problems associated with stability issues in solid drug formulations have been addressed. See, e.g., U.S. Pat. No. 7,772,273 (LifeCyclePharma), U.S. Pat. No. 6,680,341 (LEK), U.S. Pat. No. 6,631,505 (LEK), US 2010/0178338 (Ranbaxy); and U.S. Patent Application Publication Nos. US 2009/0264487 (LEK) and US 2009/0247603 (Orbus Pharma).

Administration of one active agent and/or diet modification may not be sufficient to reach a patient's target cholesterol and/or lipid levels. There remains a need in the art for compositions and/or methods to better regulate abnormal plasma lipid values in subjects in need of such treatment. Such compositions must also be sufficiently stable for pharmaceutical use and provide for sufficient solubilization, digestion, bioavailability and/or absorption of omega-3 fatty acids in vivo while maintaining the ability to cross cell membranes.

It is to be understood that both the foregoing general description and the following detailed description are exemplary and explanatory only and are not restrictive of the present disclosure, as claimed.

The present disclosure is directed to a pharmaceutical composition comprising: a fatty acid oil mixture comprising at least 75% eicosapentaenoic acid (EPA) and docosahexaenoic acid (DHA), by weight of the fatty acid oil mixture, wherein the EPA and DHA are in a form chosen from ethyl ester and triglyceride; at least one free fatty acid; and at least one statin or pharmaceutically acceptable salt; hydrate, solvate, or complex thereof.

The present disclosure is also directed to a pharmaceutical preconcentrate comprising: a fatty acid oil mixture comprising at least 75% eicosapentaenoic acid (EPA) and docosahexaenoic acid (DHA), by weight of the fatty acid oil mixture, wherein the EPA and DHA are in a form chosen from ethyl ester and triglyceride; at least one free fatty acid; at least one surfactant; and at least one statin or pharmaceutically acceptable salt, hydrate, solvate, or complex thereof.

For example, the present disclosure provides for a pharmaceutical preconcentrate comprising: a fatty acid oil mixture comprising from about 80% to about 88% eicosapentaenoic acid (EPA) and docosahexaenoic acid (DHA), by weight of the fatty acid oil mixture, wherein the EPA and DHA are in ethyl ester form; at least one free fatty acid comprising from about 80% to about 88% eicosapentaenoic acid (EPA) and docosahexaenoic acid (DHA), by weight of the at least one free fatty acid, wherein the EPA and DHA are in free fatty acid form; at least one surfactant chosen from polysorbate 20, polysorbate 80, and mixtures thereof; and at least one statin chosen from atorvastatin, rosuvastatin, simvastatin, and a pharmaceutically acceptable salt, hydrate, solvate, or complex thereof.

Further for example, the present disclosure provides for a pharmaceutical preconcentrate comprising: from about 45% to about 70% by weight, relative to the weight of the preconcentrate, of a fatty acid oil mixture comprising from about 80% to about 88% eicosapentaenoic acid (EPA) and docosahexaenoic acid (DHA) by weight of the fatty acid oil mixture, wherein the EPA and DHA are in a form chosen from ethyl ester and triglyceride; from about 5% to about 20% of at least one free fatty acid, by weight relative to the weight of the preconcentrate; from about 10% to about 45% of at least one surfactant, by weight relative to the weight of the preconcentrate; and from about 0.5% to about 15% of at least one statin or pharmaceutically acceptable salt, hydrate, solvate, or complex thereof by weight relative to the weight of the preconcentrate.

The present disclosure is also directed to a pharmaceutical preconcentrate comprising: from about 45% to about 55% by weight, relative to the weight of the preconcentrate, of a fatty acid oil mixture comprising from about 80% to about 88% eicosapentaenoic acid (EPA) and docosahexaenoic acid (DHA) by weight of the fatty acid oil mixture, wherein the EPA and DHA are in a form chosen from ethyl ester and triglyceride; from about 10% to about 15% of at least one free fatty acid, by weight relative to the weight of the preconcentrate; from about 20% to about 30% of at least one surfactant, by weight relative to the weight of the preconcentrate; and from about 1% to about 10% of at least one statin or pharmaceutically acceptable salt, hyrate, solvate, or complex thereof by weight relative to the weight of the preconcentrate.

The present disclosure is further directed to a pharmaceutical preconcentrate comprising: a fatty acid oil mixture comprising from about 80% to about 88% eicosapentaenoic acid (EPA) and docosahexaenoic acid (DHA) by weight of the fatty acid oil mixture, wherein the EPA and DHA are in ethyl ester form; at least one free fatty acid comprising oleic acid; at least one surfactant chosen from polysorbate 20 and polysorbate 80; and at least one statin chosen from atorvastatin, rosuvastatin, simvastatin, and a pharmaceutically acceptable salt, hydrate, solvate, or complex thereof.

The present disclosure is also directed to a self-nanoemulsifying drug delivery system (SNEDDS), self-microemulsifying drug delivery system (SMEDDS), or self-emulsifying drug delivery system (SEDDS) comprising a pharmaceutical preconcentrate comprising: a fatty acid oil mixture comprising at least 75% eicosapentaenoic acid (EPA) and docosahexaenoic acid (DHA), by weight of the fatty acid oil mixture, wherein the EPA and DHA are in a form chosen from ethyl ester and triglyceride; at least one free fatty acid; at least one surfactant; and at least one statin or pharmaceutically acceptable salt, hydrate, solvate, or complex thereof; wherein the preconcentrate forms an emulsion in an aqueous solution.

The present disclosure further provides for a method of treating at least one health problem in a subject in need thereof comprising administering to the subject a pharmaceutical composition comprising: a pharmaceutically-effective amount of a fatty acid oil mixture comprising at least 75% eicosapentaenoic acid (EPA) and docosahexaenoic acid (DHA), by weight of the fatty acid oil mixture, wherein the EPA and DHA are in a form chosen from ethyl ester and triglyceride; at least one free fatty acid; and at least one statin or pharmaceutically acceptable salt, hydrate, solvate, or complex thereof; wherein the at least one health problem is chosen from irregular plasma lipid levels (e.g., hypertriglyceridemia, hypercholesterolemia, and/or mixed dyslipidemia), cardiovascular functions, immune functions, visual functions, insulin action, neuronal development, heart failure, and post myocardial infarction. For example, the composition further comprises at least one surfactant to form a pharmaceutical preconcentrate, such as, the preconcentrate forms a self-nanoemulsifying drug delivery system (SNEDDS), self-microemulsifying drug delivery system (SMEDDS), or self-emulsifying drug delivery system (SEDDS) in an aqueous solution.

The present disclosure is also directed to a method for enhancing at least one parameter chosen from hydrolysis, solubility, bioavailability, absorption, and combinations thereof of eicosapentaenoic acid (EPA) and docosahexaenoic acid (DHA) comprising combining: a fatty acid oil mixture comprising EPA and DHA in a form chosen from ethyl ester and triglyceride; at least one free fatty acid; and at least one statin or pharmaceutically acceptable salt, hydrate, solvate, or complex thereof, For example, a method for enhancing at least one parameter chosen from hydrolysis, solubility, bioavailability, absorption, and combinations thereof of eicosapentaenoic acid (EPA) and docosahexaenoic acid (DHA) comprising combining: a fatty acid oil mixture comprising EPA and DHA in a form chosen from ethyl ester and triglyceride; at least one free fatty acid; at least one surfactant; and at least one statin or pharmaceutically acceptable salt, hydrate, solvate, or complex thereof; wherein the fatty acid oil mixture, the at least one free fatty acid, the at least one surfactant, and the at least one statin or pharmaceutically acceptable salt, hydrate, solvate, or complex thereof form a preconcentrate. In addition, the preconcentrate forms a self-nanoemulsifying drug delivery system (SNEDDS), self-microemulsifying drug delivery system (SMEDDS), or self-emulsifying drug delivery system (SEDDS) in an aqueous solution.

In a further embodiment, the present disclosure is directed to a pharmaceutical composition comprising a fatty acid oil mixture comprising at least 75% eicosapentaenoic acid (EPA) and docosahexaenoic acid (DHA), by weight of the fatty acid oil mixture, wherein the EPA and DHA are in a form chosen from ethyl ester and triglyceride; at least one free fatty acid, and at least one statin or pharmaceutically acceptable salt, hydrate, solvate, or complex thereof for the treatment of at least one health problem chosen from irregular plasma lipid levels (e.g., hypertriglyceridemia, hypercholesterolemia and/or mixed dyslipidemia), cardiovascular functions, immune functions, visual functions, insulin action, neuronal development, heart failure, and post myocardial infarction.

In yet still a further embodiment, the present disclosure provides for a pharmaceutical preconcentrate comprising a fatty acid oil mixture comprising at least 75% eicosapentaenoic acid (EPA) and docosahexaenoic acid (DHA), by weight of the fatty acid oil mixture, wherein the EPA and DHA are in a form chosen from ethyl ester and triglyceride; at least one free fatty acid; at least one surfactant; and at least one statin or pharmaceutically acceptable salt, hydrate, solvate, or complex thereof for the treatment of at least one health problem chosen from irregular plasma lipid levels (e.g., hypertriglyceridemia, hypercholesterolemia and/or mixed dyslipidemia), cardiovascular functions, immune functions, visual functions, insulin action, neuronal development, heart failure, and post myocardial infarction.

The present disclosure is also directed to a self-nanoemulsifying drug delivery system (SNEDDS), self-microemulsifying drug delivery system (SMEDDS), or self-emulsifying drug delivery system (SEDDS) comprising a pharmaceutical preconcentrate comprising: a fatty acid oil mixture comprising at least 75% eicosapentaenoic acid (EPA) and docosahexaenoic acid (DHA), by weight of the fatty acid oil mixture, wherein the EPA and DHA are in a form chosen from ethyl ester and triglyceride; at least one free fatty acid; at least one surfactant, and at least one statin or pharmaceutically acceptable salt, hydrate, solvate, or complex thereof; wherein the preconcentrate forms an emulsion in an aqueous solution for the treatment of at least one health problem chosen from irregular plasma lipid levels levels (e.g., hypertriglyceridemia, hypercholesterolemia and/or mixed dyslipidemia), cardiovascular functions, immune functions, visual functions, insulin action, neuronal development, heart failure, and post myocardial infarction.

### BRIEF DESCRIPTION OF THE DRAWINGS

FIG. 1 shows biosynthesis of cholesterol and a mechanism of action of statins (Jo Klaveness, Compendium in Medicinal Chemistry, Oslo, Norway (2009)).

FIG. 2 shows the chemical formulae of simvastatin, lovastatin, pravastatin, fluvastatin, and atorvastatin.

FIG. 3 shows the viscosity of preconcentrates A-L.

FIG. 4 shows the average particle size distribution for preconcentrates A-F, I, and J in gastric media and intestinal media.

FIG. 5 shows the read out from the Malvern zetasizer for four consecutive measurements on preconcentrate A in gastric media.

FIG. 6 shows the read out from the Malvern zetasizer for four consecutive measurements on preconcentrate B in gastric media.

FIG. 7 shows the read out from the Malvern zetasizer for four consecutive measurements on preconcentrate C in gastric media.

FIG. 8 shows the read out from the Malvern zetasizer for four consecutive measurements on preconcentrate in gastric media.

FIG. 9 shows the read out from the Malvern zetasizer for four consecutive measurements on preconcentrate E in gastric media.

FIG. 10 shows the read out from the Malvern zetasizer for four consecutive measurements on preconcentrate F in gastric media.

FIG. 11 shows the read out from the Malvern zetasizer for four consecutive measurements on preconcentrate I in gastric media.

FIG. 12 shows the read out from the Malvern zetasizer for four consecutive measurements on preconcentrate J in gastric media.

FIG. 13 shows the read out from the Malvern zetasizer for four consecutive measurements on preconcentrate A in intestinal media.

FIG. 14 shows the read out from the Malvern zetasizer for four consecutive measurements on preconcentrate B in intestinal media.

FIG. 15 shows the read out from the Malvern zetasizer for four consecutive measurements on preconcentrate C in intestinal media.

FIG. 16 shows the read out from the Malvern zetasizer for four consecutive measurements on preconcentrate D in intestinal media.

FIG. 17 shows the read out from the Malvern zetasizer for four consecutive measurements on preconcentrate E in intestinal media.

FIG. 18 shows the read out from the Malvern zetasizer for four consecutive measurements on preconcentrate F in intestinal media.

FIG. 19 shows the read out from the Malvern zetasizer for four consecutive measurements on preconcentrate I in intestinal media.

FIG. 20 shows the read out from the Malvern zetasizer for four consecutive measurements on preconcentrate J in intestinal media.

FIG. 21 shows the disappearance of EPA-EE and DHA-EE and the appearance of EPA-FA and DHA-FA during lipolysis of Omacor®

FIG. 22 shows the percent recovery of EPA+DHA at different time-points for Omacor®.

FIG. 23 shows the percent lipolysis of EPA-EE, DHA-EE and total K85EE at different time points for Omacor®.

FIG. 24 shows the disappearance of EPA-EE and DHA-EE and the appearance of EPA-FA and DHA-FA during lipolysis of preconcentrate A.

FIG. 25 shows the percent recovery of EPA+DHA at different time-points for preconcentrate A.

FIG. 26 shows the percent lipolysis of EPA-EE, DHA-EE and total K85EE at different time points for preconcentrate A.

FIG. 27 shows the disappearance of EPA-EE and DHA-EE and the appearance of EPA-FA and DHA-FA during lipolysis of preconcentrate B.

FIG. 28 shows the percent recovery of EPA+DHA at different time-points for preconcentrate B.

FIG. 29 shows the percent lipolysis of EPA-EE, DHA-EE and total K85EE at different time points for preconcentrate B.

FIG. 30 shows the disappearance of EPA-EE and DHA-EE and the appearance of EPA-FA and DHA-FA during lipolysis of preconcentrate C.

FIG. 31 shows the percent recovery of EPA+DHA at different time-points for preconcentrate C.

FIG. 32 shows the percent lipolysis of EPA-EE, DHA-EE and total K85EE at different time points for preconcentrate C.

FIG. 33 shows the disappearance of EPA-EE and DHA-EE and the appearance of EPA-FA and DHA-FA during lipolysis of preconcentrate D.

FIG. 34 shows the percent recovery of EPA+DHA at different time-points for preconcentrate D.

FIG. 35 shows the percent lipolysis of EPA-EE, DHA-EE and total K85EE at different time points for preconcentrate D.

FIG. 36 shows the disappearance of EPA-EE and DHA-EE and the appearance of EPA-FA and DHA-FA during lipolysis preconcentrate E.

FIG. 37 shows the percent recovery of EPA+DHA at different e-points for preconcentrate E.

FIG. 38 shows the percent lipolysis of EPA-EE, DHA-EE and total K85EE at different time points for preconcentrate E.

FIG. 39 shows the plasma concentration versus time profile of the total lipid concentration of EPA for Example 14.

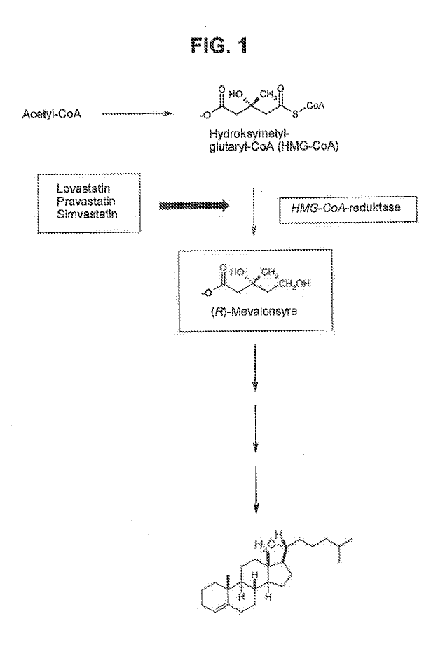

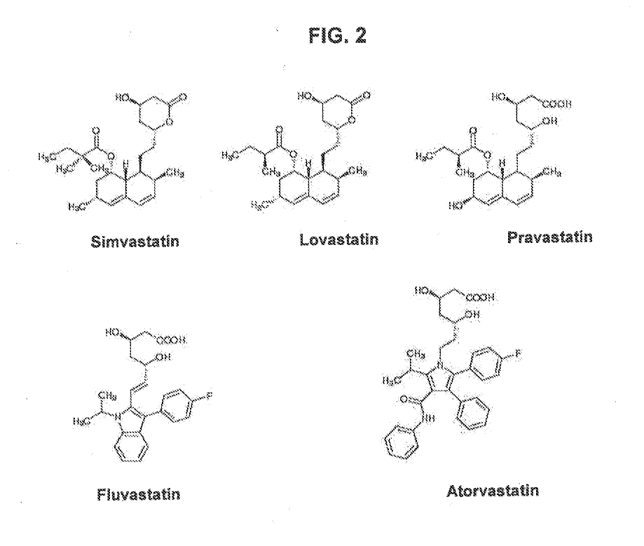

### DESCRIPTION

Particular aspects of the disclosure are described in greater detail below. The terms and definitions as used in the present application and as clarified herein are intended to represent the meaning within the present disclosure. The patent and scientific literature referred to herein is hereby incorporated by reference, The terms and definitions provided herein control, if in conflict with terms and/or definitions incorporated by reference.

The singular forms “a,” “an,” and “the” include plural reference unless the context dictates otherwise.

The terms “approximately” and “about” mean to be nearly the same as a referenced number or value. As used herein, the terms “approximately” and “about” should be generally understood to encompass ±10% of a specified amount, frequency or value.

The terms “administer,” “administration” or “administering” as used herein refer to (1) providing, giving, dosing and/or prescribing by either a health practitioner or his authorized agent or under his direction a composition according to the disclosure, and (2) putting into, taking or consuming by the patient or person himself or herself, a composition according to the disclosure.

The present disclosure provides for pharmaceutical compositions comprising a fatty acid oil mixture, at least one free fatty acid, and at least one statin or pharmaceutically acceptable salt, hydrate, solvate, or complex thereof, and methods of use thereof. The compositions can further comprise at least one surfactant to form a preconcentrate. The preconcentrates of the present disclosure can produce dispersions of low or very low mean particle size when mixed with an aqueous medium. Such dispersions can be characterized as nanoemulsions, microemulsions, or emulsions. For example, upon delivery, the preconcentrates are thought to produce dispersions with gastric or other physiological fluids generating self-nanoemulsifying drug delivery systems (SNEDDS), self-microemulsifying drug delivery systems (SMEDDS), or self emulsifying drug delivery systems (SEDDS).

Fatty Acid Oil Mixture

Compositions of the present disclosure comprise at least one fatty acid oil mixture. The fatty acid oil mixture comprises eicosapentaenoic acid (EPA) and docosahexaenoic acid (DHA). As used herein, the term “fatty acid oil mixture” includes fatty acids, such as unsaturated (e.g., monounsaturated, polyunsaturated) or saturated fatty acids, as well as pharmaceutically-acceptable esters, free acids, mono-, di- and triglycerides, derivatives, conjugates, precursors, salts, and mixtures thereof. In at least one embodiment, the fatty acid oil mixture comprises fatty acids, such as omega-3 fatty acids, in a form chosen from ethyl ester and triglyceride.

The term “omega-3 fatty acids” includes natural and synthetic omega-3 fatty acids, as well as pharmaceutically-acceptable esters, free acids, triglycerides, derivatives, conjugates (see, e.g., Zaloga et al., U.S. Patent Application Publication No. 2004/0254357, and Horrobin et al., U.S. Pat. No. 6,245,811, each hereby incorporated by reference), precursors, salts, and mixtures thereof. Examples of omega-3 fatty acid oils include, but are not limited to, omega-3 polyunsaturated, long-chain fatty acids such as eicosapentaenoic acid (EPA), docosahexaenoic acid (DHA), α-linolenic acid (ALA), heneicosapentaenoic acid (HPA), docosapentaenoic acid (DPA), eicosatetraenoic acid (ETA), eicosatrienoic acid (ETE), and octadecatetraenoic acid (i.e., stearidonic acid, STA); esters of omega-3 fatty acids with glycerol such as mono-, di- and triglycerides, and esters of the omega-3 fatty acids and a primary, secondary and/or tertiary alcohol, such as, for example, fatty acid methyl esters and fatty acid ethyl esters. The omega-3 fatty acids, esters, triglycerides, derivatives, conjugates, precursors, salts and/or mixtures thereof according to the present disclosure can be used in their pure form and/or as a component of an oil, for example, as marine oil (e.g., fish oil and purified fish oil concentrates), algae oils, microbial oils and plant-based oils.

In some embodiments of the present disclosure, the fatty acid oil mixture comprises EPA and DHA. Further for example, the fatty acid oil mixture comprises EPA and DHA in a form chosen from ethyl ester and triglyceride.

The fatty acid oil mixture of the present disclosure may further comprise at least one fatty acid other than EPA and DHA. Examples of such fatty acids include, but are not limited to, omega-3 fatty acids other than EPA and DHA and omega-6 fatty acids. For example, in some embodiments of the present disclosure, the fatty acid oil mixture comprises at least one fatty acid other than EPA and DHA chosen from α-linolenic acid (ALA), heneicosapentaenoic acid (HPA), docosapentaenoic acid (DPA), eicosatetraenoic acid (ETA), eicosatrienoic acid (ETE), and stearidonic acid (STA). In some embodiments, the at least one fatty acid other than EPA and DHA is chosen from linoleic acid, gamma-linolenic acid (GLA), arachidonic acid (AA), docosapentaenoic acid (i.e., osbond acid), and mixtures thereof. In some embodiments, the at least one fatty acid other than EPA and DHA is in a form chosen from ethyl ester and triglyceride.

Examples of further fatty acids, or mixtures thereof (fatty acid oil mixtures) encompassed by the present disclosure include, but are not limited to, the fatty acids defined in the European Pharamacopoeia Omega-3 Ethyl Esters 90 or the USP omega-3 EE Monograph. Commercial embodiments provide for various omega-3 fatty acids, combinations, and other components as a result of the transesterification process or method of preparation in order to obtain the omega-3 fatty acid(s) from various sources, such as marine, algae, microbial, and plant-based sources.

The fatty acid oil mixture according to the present disclosure may be derived from animal oils and/or non-animal oils. In some embodiments of the present disclosure, the fatty acid oil mixture is derived from at least one oil chosen from marine oil, algae oil, plant-based oil, and microbial oil. Marine oils include, for example, fish oil, krill oil, and lipid composition derived from fish. Plant-based oils include, for example, flaxseed oil, canola oil, mustard seed oil, and soybean oil. Microbial oils include, for example, products by Martek. In at least one embodiment of the present disclosure, the fatty acid oil mixture is derived from a marine oil, such as a fish oil. In at least one embodiment, the marine oil is a purified fish oil.

In some embodiments of the present disclosure, the fatty acids, such as omega-3 fatty acids, of the fatty acid oil mixture are esterified, such as alkyl esters and further for example, ethyl ester. In other embodiments, the fatty acids are chosen from mono-, di-, and triglycerides.

In some embodiments, the fatty acid oil mixture is obtained by a transesterification of the body oil of a fat fish species coming from, for example, anchovy or tuna oil, and subsequent physico-chemical purification processes, including urea fractionation followed by molecular distillation. In some embodiments, the crude oil mixture may also be subjected to a stripping process for decreasing the amount of environmental pollutants and/or cholesterol before the transesterification.

In another embodiment, the fatty acid oil mixture is obtained by using supercritical CO₂ extraction or chromatography techniques, for example to up-concentrate primary EPA and DHA from fish oil concentrates.

In some embodiments of the present disclosure, at least one of the omega-3 fatty acids of the fatty acid oil mixture has a cis configuration. Examples include, but are not limited to, (all-Z)-9,12,15-octadecatrienoic acid (ALA), (all-Z)-6,9,12,15-octadecatetraenoic acid (STA), (all-Z)-11,14,17-eicosatrienoic acid (ETE), (all-Z)-5,8,11,14,17-eicosapentaenoic acid (EPA), (all-Z)-4,7,10,13,16,19-docosahexaenoic acid (DHA), (all-Z)-8,11,14,17-eicosatetraenoic acid (ETA), (all-Z)-7,10,13,16,19-docosapentaenoic acid (DPA), (all-Z)-6,9,12,15,19-heneicosapentaenoic acid (HPA); (all-Z)-5,8,11,14-eicosatetraenoic acid, (all-Z)-4,7,10,13,16-docosapentaenoic acid (osbond acid), (all-Z)-9,12-octadecadienoic acid (linoleic acid), (all-Z)-5,8,11,14-eicosatetraenoic acid (AA), (all-Z)-6,9,12-octadecatrienoic acid (GLA); (Z)-9-octadecenoic acid (oleic acid), 13(Z)-docosenoic acid (erucic acid), (R-(Z))-12-hydroxy-9-octadecenoic acid (ricinoleic acid).

In some embodiments of the present disclosure, the weight ratio of EPA:DHA of the fatty acid oil mixture ranges from about 1:10 to about 10:1, from about 1:8 to about 8:1, from about 1:6 to about 6:1, from about 1:5 to about 5:1, from about 1:4 to about 4:1, from about 1:3 to about 3:1, or from about 1:2 to 2 about:1. In at least one embodiment, the weight ratio of EPA:DHA of the fatty acid oil mixture ranges from about 1:2 to about 2:1. In at least one embodiment, the weight ratio of EPA:DHA of the fatty acid oil mixture ranges from about 1:1 to about 2:1. In at least one embodiment, the weight ratio of EPA:DHA of the fatty acid oil mixture ranges from about 1.2 to about 1.3.

Free Fatty Acid (FFA)

The compositions presently disclosed comprise at least one free fatty acid. Without being bound by theory, it is believed that the addition of at least one free fatty acid may enhance or improve lipolysis of the fatty acid oil mixture in the body, e.g., the interconversion of fatty acid esters and/or triglycerides to the free fatty acid form for efficient uptake. The addition of at least one free fatty acid may, for example, provide for enhanced or improved hydrolysis, solubility, bioavailability, absorption, or any combinations thereof of fatty acids of the fatty acid oil mixture in vivo.

Examples of free fatty acids include, but are not limited to, EPA, DHA, α-linolenic acid (ALA), heneicosapentaenoic acid (HPA), docosapentaenoic acid (DPA), eicosatetraenoic acid (ETA), eicosatrienoic acid (ETE), stearidonic acid (STA), linoleic acid, gamma-linolenic acid (GLA), arachidonic acid (AA), osbond acid, oleic acid, ricinoleic acid, erucic acid, and mixtures thereof. In at least one embodiment, the at least one free fatty acid is a polyunsaturated fatty acid.

In some embodiments, the at least one free fatty acid is chosen from oleic acid, ricinoleic acid, linoleic acid, and erucic acid. In one embodiment, the at least one free fatty acid comprises oleic acid or linoleic acid.

In some embodiments, the at least one free fatty acid comprises at least 80% omega-3 fatty acids by weight of the at least one free fatty acid, such as at least 90% omega-3 fatty acids by weight of the at least one free fatty acid.

In some embodiments, the at least one free fatty acid comprises at least 75% EPA and DHA by weight of the at least one free fatty acid. For example, in some embodiments, the at least one free fatty acid comprises at least 80% by weight, at least 85% by weight, at least 90% by weight, or at least 95% EPA and DHA, by weight of the at least one free fatty acid. In some embodiments, the at least one free fatty acid comprises about 80% EPA and DHA by weight of the at least one free fatty acid, such as about 85%, about 90%, about 95%, or any number in between, by weight of the at least one free fatty acid. The at least one free fatty acid can be used in a pure form and/or as a component of an oil, for example, as marine oil (e.g., fish oil and purified fish oil concentrates), microbial oil and plant-based oils.

In some embodiments, the at least one free fatty acid comprises from about 75% to about 95% EPA and DHA by weight of the at least one free fatty acid, such as from about 75% to about 90%, from about 75% to about 85%, from about 75% to about 80%, from about 80% to about 95%, from about 80% to about 90%, from about 80% to about 85%, from about 85% to about 95%, from about 85% to about 90%, and further for example, from about 90% to about 95% by weight of the at least one free fatty acid, or any number in between. In at least one embodiment, the at least one free fatty acid comprises from about 80% to about 88% EPA and DHA, by weight of the at least one free fatty acid, such as from about 80% to about 85% EPA and DHA by weight, such as about 84%, by weight of the at least one free fatty acid.

Commercial embodiments of at least one free fatty acid encompassed by the present disclosure include, but are not limited to, K85FA (Pronova BioPharma Norge AS).

Statin

The compositions presently disclosed comprise at least one statin or pharmaceutically acceptable salt, hydrate, solvate, or complex thereof. As used herein, the term “statin” includes statins, pharmaceutically acceptable salts thereof, hydrates thereof, solvates thereof, and complexes thereof. Any regulatory approved statin may be suitable for the compositions, preconcentrates, and/or SNEDDS/SMEDDS/SEDDS presently disclosed. Examples include, but are not limited to, atorvastatin, cerivastatin, fluvastatin, itavastatin, lovastatin, mevastatin, rosuvastatin, simvastatin, pravastatin, and pitavastatin.

Statins according to the present disclosure may be used in the free acid form or in the form of a pharmaceutically acceptable salt, hydrate, solvate, or complex thereof. Typical salts of statins suitable for the present disclosure include, for example, ammonia salts. L-arginine salts, benethamine salts, benzathine salts, calcium salts, choline salts, deanol salts, diethanolamine salts, diethylamine salts, 2-(diethylamino)-ethanol salts, ethanolamine salts, ethylenediamine salts. N-methyl-glucamine salts, hydravamine salts, 1H-imidazole salts, L-lysine salts, magnesium salts, 4-(2-hydroxyethyl)-morpholine salts, piperazine salts, potassium salts, 1-(2-hydroxyethyl)-pyrrolidine salts, sodium salts, triethanolamine salts, tromethamine salts, zinc salts, and meglumin salts. Statins according to the present disclosure may also be in lactone form, for example simvastatin, mevastatin, and/or lovastatin. Complexes according to the present disclosure include, for example, complexes comprising a statin and at least one of meglumin CD, meglumin beta-CD, calcium CD, calcium beta-CD, crysmeb, beta cyclodextrin, and kleptose. In some embodiments, the statin complex may be crystallized.

In some embodiments of the present disclosure, the at least one statin is chosen from atorvastatin, cerivastatin, fluvastatin, itavastatin, lovastatin, mevastatin, rosuvastatin, simvastatin, pravastatin, pitavastatin, pharmaceutically acceptable salts, hydrates, solvates, and complexes thereof. For example, in some embodiments, the at least one statin is chosen from simvastatin, atorvastatin, rosuvastatin, and pharmaceutically acceptable salts, hydrates, solvates, and complexes thereof. In at least one embodiment, the at least one statin is chosen from atorvastatin, for example atorvastatin calcium, rosuvastatin, for example rosuvastatin calcium, and simvastatin.

Commercial embodiments of statins encompassed by the present disclosure include, but are not limited to, Lipitor® (atorvastatin), Lescol® (fluvastatin), Mevacor® (lovastatin), Crestor® (rosuvastatin), Zocor® (simvastatin), Pravachol® (pravastatin), and Livalo® (pitavastatin), or regulatory approved generics thereof.

The statins according to the present disclosure may be amorphous or in crystalline form. In at least one embodiment, the at least one statin is in amorphous form.

The amount of the at least one statin in the compositions and/or preconcentrates presently disclosed may range from about 0.1 mg to about 100 mg, such as from about 5 mg to about 80 mg, from about 10 mg to about 80 mg, or from about 10 mg to about 40 mg. In at least one embodiment, the at least one statin is chosen from atorvastatin, such as atorvastatin calcium, rosuvastatin, such as rosuvastatin calcium, and simvastatin, in an amount ranging from about 10 mg to about 80 mg.

In some embodiments of the present disclosure, the fatty acid oil mixture acts as an active pharmaceutical ingredient (API), i.e., the at least one statin or pharmaceutically acceptable salt, hydrate, solvate, or complex thereof and the fatty acid oil mixture both act as APIs. For example, the present disclosure provides for a pharmaceutical composition comprising a fatty acid oil mixture, at least one free fatty acid, and at least one statin or pharmaceutically acceptable salt, hydrate, solvate, or complex thereof. In some embodiments, the fatty acid oil mixture is present in a pharmaceutically-acceptable amount. As used herein, the term “pharmaceutically-effective amount” means an amount sufficient to treat, e.g., reduce and/or alleviate the effects, symptoms, etc., at least one health problem in a subject in need thereof. In at least some embodiments of the present disclosure, the fatty acid oil mixture does not comprise an additional active agent. For example, in some embodiments, the pharmaceutical composition comprises a fatty acid oil mixture, at least one free fatty acid, and at least one statin or pharmaceutically acceptable salt, hydrate, solvate, or complex thereof, wherein the fatty acid oil mixture and the statin are the sole active agents in the composition.

In the pharmaceutical compositions presently disclosed, the fatty acid oil mixture may comprise at least 75% EPA and DHA by weight of the fatty acid oil mixture. For example, in one embodiment, the fatty acid oil mixture comprises at least 80% EPA and DHA by weight of the fatty acid oil mixture, such as at least 85%, at least 90%, or at least 95%, by weight of the fatty acid oil mixture. In some embodiments, the fatty acid oil mixture comprises about 80% EPA and DHA by weight of the fatty acid oil mixture, such as about 85%, about 90%, about 95%, or any number in between, by weight of the fatty acid oil mixture.

For example, in some embodiments, the fatty acid oil mixture comprises from about 75% to about 95% EPA and DHA by weight of the fatty acid oil mixture, such as from about 75% to about 90%, from about 75% to about 88%, from about 75% to about 85%, from about 75% to about 80%, from about 80% to about 95%, from about 80% to about 90%, from about 80% to about 85%, from about 85% to about 95%, from about 85% to about 90%, and further for example, from about 90% to about 95% EPA and DHA, by weight of the fatty acid oil mixture, or any number in between. In at least one embodiment, the fatty acid oil mixture comprises from about 80% to about 85% EPA and DHA, by weight of the fatty acid oil mixture, such as from about 80% to about 88%, such as about 84%, by weight of the fatty acid oil mixture.

in some embodiments, the fatty acid oil mixture comprises at least 95% of EPA or DHA, or EPA and DHA, by weight of the fatty acid oil mixture, wherein the EPA and DHA are in ethyl ester form.

In a further embodiment, the fatty acid oil mixture may comprise other omega-3 fatty acids. For example, the present disclosure encompasses at least 90% omega-3 fatty acids, by weight of the fatty acid oil mixture.

In one embodiment, for example, the fatty acid oil mixture comprises from about 75% to about 88% EPA and DHA, by weight of the fatty acid oil mixture, wherein the EPA and DHA are in ethyl ester form; wherein the fatty acid oil mixture comprises at least 90% of omega-3 fatty acids in ethyl ester form, by weight of the fatty acid oil mixture.

In another embodiment, the fatty acid oil mixture comprises from about 75% to about 88% EPA and DHA, by weight of the fatty acid oil mixture, wherein the EPA and DHA are in ethyl ester form; wherein the fatty acid oil mixture comprises at least 90% of omega-3 fatty acids in ethyl ester form, by weight of the fatty acid oil mixture, and wherein the fatty acid oil mixture comprises α-linolenic acid (ALA).

In one embodiment, the fatty acid oil mixture comprises from about 80% to about 88% EPA and DNA by weight of the fatty acid oil mixture, wherein the EPA and DNA are in ethyl ester form, and further comprises docosapentaenoic acid (DPA) in ethyl ester form.

In another embodiment, the fatty acid oil mixture comprises from about 80% to about 88% EPA and DNA by weight of the fatty acid oil mixture, wherein the EPA and DHA are in ethyl ester form, and further comprises from about 1% to about 4% (all-Z omega-3)-6,9,12,15,18-heneicosapentaenoic acid (HPA) in ethyl ester form, by weight of the fatty acid oil mixture.

In another embodiment, the fatty acid oil mixture comprises from about 80% to about 88% EPA and DNA by weight of the fatty acid oil mixture, wherein the EPA and DNA are in ethyl ester form; and from 1% to about 4% fatty acid ethyl esters other than EPA and DNA, by weight of the fatty acid oil mixture, wherein the fatty acid ethyl esters other than EPA and DNA have C₂₀, C₂₁, or C₂₂ carbon atoms.

In one embodiment, the fatty acid oil mixture may comprise K85EE or AGP 103 (Pronova BioPharma Norge AS). In another embodiment, the fatty acid oil mixture may comprise K85TG (Pronova BioPharma Norge AS).

In one embodiment, the pharmaceutical composition comprising at least K85EE, K85-FA, and Tween 20 or 80, for example, provide for enhanced bioavailability. For example, the bioavailability may be increased >about 40%, such as, about 80%.

In one embodiment, the pharmaceutical composition comprises K85EE, K85-FA, and at least one surfactant chosen from Tween-20 and Tween-80 in a fixed dose combination with atorvastatin (Lipitor®).

In another embodiment, the pharmaceutical composition comprises K85EE, oleic acid, and Tween-20 in a fixed dose combination with atorvastatin (Lipitor®).

EPA and DHA Products

In at least one embodiment, the fatty acid oil mixture comprises at least 75% EPA and DHA by weight of the fatty acid oil mixture, of which at least 95% is EPA. In another embodiment, the fatty acid oil mixture comprises at least 80% EPA and DNA by weight of the fatty acid oil mixture, of which at least 95% is EPA. In yet another embodiment, the fatty acid oil mixture comprises at least 90% EPA and DHA by weight of the fatty acid oil mixture, of which at least 95% is EPA.

In another embodiment, the fatty acid oil mixture comprises at least 75% EPA and DHA by weight of the fatty acid oil mixture, of which at least 95% is DHA. For example, in one embodiment, the fatty acid oil mixture comprises at least 80% EPA and DHA by weight of the fatty acid oil mixture, of which at least 95% is DHA. In another embodiment, the fatty acid oil mixture comprises at least 90% EPA and DHA by weight of the fatty acid oil mixture, of which at least 95% is DHA.

In some embodiments, the fatty acid oil mixture comprises from about 50% to about 95% by weight and the at least one free fatty acid comprises from about 5% to about 50% by weight, each relative to the total weight of the composition.

Additional Oils

In some embodiments, preconcentrates of the present disclosure further comprise at least one additional oil, such as medium chain triglyceride (MCT) oil and long chain triglyceride (LCT) oil, including sesame oil. Further examples can include ethyl oleate.

Superdisintegrant

The compositions presently disclosed may further comprise at least one superdistintegrant. Superdisintegrants may, for example, improve disintegrant efficiency resulting in decreased use levels in comparison to traditional disintegrants. Examples of superdisintegrants include, but are not limited to, crosscarmelose (a crosslinked cellulose), crospovidone (a crosslinked polymer), sodium starch glycolate (a crosslinked starch), and soy polysaccharides. Commercial examples of superdisintegrants include Kollidon® (BASF), Polyplasdone® XL (ISP), and Ac-Di-Sol (FMC BioPolymer).

In some embodiments of the present disclosure, the composition comprises from about 1% to about 25% of at least one superdisintegrant by weight of the composition, such as from about 1% to about 20% by weight, or from about 1% to about 15% by weight of the composition. In some embodiments, the compositions comprising at least one superdisintegrant are in a tablet form.

Surfactant/Preconcentrate

The present disclosure further provides for a preconcentrate composition. In some embodiments of the present disclosure, the composition further comprises at least one surfactant to form a preconcentrate. As used herein, the term “preconcentrate” refers to a composition comprising at least the combination of a fatty acid oil mixture, at least one free fatty acid, and at least one surfactant. In some embodiments, for example, the preconcentrate comprises a fatty acid oil mixture, at least one free fatty acid, at least one surfactant, and at least one statin or pharmaceutically acceptable salt, hydrate, solvate, or complex thereof.

A surfactant may, for example, lower the surface tension of a liquid or the surface tension between two liquids. For example, surfactants according to the present disclosure may lower the surface tension between the fatty acid oil mixture and/or the at least one free fatty acid and an aqueous solution.

Chemically speaking, surfactants are molecules with at least one hydrophilic part and at least one hydrophobic (i.e., lipophilic) part. Surfactant properties may be reflected in the hydrophilic-lipophilic balance (HLB) value of the surfactant, wherein the HLB value is a measure of the degree of hydrophilic versus lipophilic properties of a surfactant. The HLB value normally ranges from 0 to 20, where a HLB value of 0 represents high hydrophilic character, and a HLB of 20 represents high lipophilic character. Surfactants are often used in combination with other surfactants, wherein the HLB values are additive. The HLB value of surfactant mixtures may be calculated as follows:

HLB_{A}(fraction of surfactant A)+HLB_{B}(fraction of surfactant B)=HLB_{A+B mixture}

Surfactants are generally classified as ionic surfactants, e.g., anionic or cationic surfactants, and nonionic surfactants. If the surfactant contains two oppositely charged groups, the surfactant is named a zwitterionic surfactant. Other types of surfactants include, for example, phospholipids.

In at least one embodiment of the present disclosure, the composition comprises at least one surfactant chosen from nonionic, anionic, cationic, and zwitterionic surfactants.

Non-limiting examples of nonionic surfactants suitable or the present disclosure are mentioned below.

Pluronic® surfactants are nonionic copolymers composed of a central hydrophobic polymer (polyoxypropylene(poly(propylene oxide))) with a hydrophilic polymer (polyoxyethylene(poly(ethylene oxide))) on each side. Various commercially-available Pluronic® products are listed in Table 1.

TABLE 1
Examples of Pluronic ® surfactants.

| | | Average Molecular | |
|---|---|---|---|
| | Type | Weight (D) | HLB Value |
| Pluronic ® L-31 | Non-ionic | 1100 | 1.0-7.0 |
| Pluronic ® L-35 | Non-ionic | 1900 | 18.0-23.0 |
| Pluronic ® L-61 | Non-ionic | 2000 | 1.0-7.0 |
| Pluronic ® L-81 | Non-ionic | 2800 | 1.0-7.0 |
| Pluronic ® L-64 | Non-ionic | 2900 | 12.0-18.0 |
| Pluronic ® L-121 | Non-ionic | 4400 | 1.0-7.0 |
| Pluronic ® P-123 | Non-ionic | 5800 | 7-9 |
| Pluronic ® F-68 | Non-ionic | 8400 | >24 |
| Pluronic ® F-108 | Non-ionic | 14600 | >24 |

Brij® are nonionic surfactants comprising polyethylene ethers. Various commercially-available Brij® products are listed in Table 2.

TABLE 2
Examples of Brij ® surfactants.

| | | | HLB |
|---|---|---|---|
| | Type | Compound | Value |
| Brij ® 30 | Non-ionic | polyoxyethylene(4) lauryl ether | 9.7 |
| Brij ® 35 | Non-ionic | polyoxyethylene (23) lauryl ether | 16.9 |
| Brij ® 52 | Non-ionic | polyoxyethylene (2) cetyl ether | 5.3 |
| Brij ® 56 | Non-ionic | polyoxyethylene (10) cetyl ether | 12.9 |
| Brij ® 58 | Non-ionic | polyoxyethylene (20) cetyl ether | 15.7 |
| Brij ® 72 | Non-ionic | polyoxyethylene (2) stearyl ether | 4.9 |
| Brij ® 76 | Non-ionic | polyoxyethylene (10) stearyl ether | 12.4 |
| Brij ® 78 | Non-ionic | polyoxyethylene (20) stearyl ether | 15.3 |
| Brij ® 92V | Non-ionic | polyoxyethylene (2) oleyl ether | 4.9 |
| Brij ® 93 | Non-ionic | polyoxyethylene (2) oleyl ether | 4 |
| Brij ® 96V | Non-ionic | polyethylene glycol oleyl ether | 12.4 |
| Brij ® 97 | Non-ionic | polyoxyethylene (10) oleyl ether | 12 |
| Brij ® 98 | Non-ionic | polyoxyethylene (20) oleyl ether | 15.3 |
| Brij ® 700 | Non-ionic | polyoxyethylene (100) stearyl ether | 18 |

Span® are nonionic surfactants comprising sorbitan esters. Span® is available from different sources including Aldrich. Various commercially-available Span® products are listed in Table 3.

TABLE 3
Examples of Span ® surfactants.

| | Type | Compound | HLB Value |
|---|---|---|---|
| Span ® 20 | Non-ionic | sorbitan monolaurate | 8.6 |
| Span ® 40 | Non-ionic | sorbitan monopalmitate | 6.7 |
| Span ® 60 | Non-ionic | sorbitan monostearate | 4.7 |
| Span ® 65 | Non-ionic | sorbitan tristearate | 2.1 |
| Span ® 80 | Non-ionic | sorbitan monooleate | 4.3 |
| Span ® 85 | Non-ionic | sorbitan trioleate | 1.8 |

Tween® (polysorbates) are nonionic surfactants comprising polyoxyethylene sorbitan esters. Various commercially-available Tween® products are listed in Table 4.

TABLE 4
Examples of Tween ® surfactants.

| | Type | Compound | HLB Value |
|---|---|---|---|
| Tween ® 20 | Non-ionic | polyoxyethylene (20) | 16.0 |
| | | sorbitan monolaurate | |
| Tween ® 40 | Non-ionic | polyoxyethylene (20) | 15.6 |
| | | sorbitan monopalmitate | |
| Tween ® 60 | Non-ionic | polyoxyethylene sorbitan | 14.9 |
| | | monostearate | |
| Tween ® 65 | Non-ionic | polyoxyethylene sorbitan | 10.5 |
| | | tristearate | |
| Tween ® 80 | Non-ionic | polyoxyethylene(20) | 15.0 |
| | | sorbitan monooleate | |
| Tween ® 85 | Non-ionic | polyoxyethylene sorbane | 11.0 |
| | | trioleate | |

Myrj® are nonionic surfactants comprising polyoxyethylene fatty acid esters. Various commercially-available Myrj® products are listed in Table 5.

TABLE 5
Examples of Myrj ® surfactants.

| | Type | Compound | HLB Value |
|---|---|---|---|
| Myrj ® 45 | Non-ionic | polyoxyethylene monostearate | 11.1 |
| Myrj ® 49 | Non-ionic | polyoxyethylene monostearate | 15.0 |
| Myrj ® 52 | Non-ionic | polyoxyethylene monostearate | 16.9 |
| Myrj ® 53 | Non-ionic | polyoxyethylene monostearate | 17.9 |

Cremophor® are nonionic surfactants. Various commercially-available Cremophor® products are listed in Table 6.

TABLE 6
Examples of Cremophor ® surfactants.

| | Type | Compound | HLB Value |
|---|---|---|---|
| Cremophor ® REL | Non-ionic | polyoxyethylated | 2-14 |
| | | castor oil | |
| Cremophor ® RH40 | Non-ionic | hydrogenated | 14-16 |
| | | polyoxyethylated | |
| | | castor oil | |
| Cremophor ® RH60 | Non-ionic | hydrogenated | 15-17 |
| | | polyoxyethylated | |
| | | castor oil | |
| Cremophor ® RO | Non-ionic | hydrogenated | 16.1 |
| | | polyoxyethylated | |
| | | castor oil | |

According to the present disclosure, other exemplary nonionic surfactants include, but are not limited to, diacetyl monoglycerides, diethylene glycol monopalmitostearate, ethylene glycol monopalmitostearate, glyceryl behenate, glyceryl distearate, glyceryl monolinoleate, glyceryl mono-oleate, glyceryl monostearate, macrogol cetostearyl ether such as cetomacrogol 1000 and polyoxy 20 cetostearyl ether, macrogol 15 hydroxystearate (Solutol HS 15), macrogol lauril ethers such as laureth 4 and lauromacrogol 400, macrogol monomethyl ethers, macrogol oleyl ethers such as polyoxyl 10 oleyl ether, macrogol stearates such as polyoxyl 40 stearate, menfegol, mono and diglycerides, nonoxinols such as nonoxinol-9, nonoxinol-10 and nonoxinol-11, octoxinols such as octoxinol 9 and oxtoxinol 10, polyoxamers such as polyoxalene, polyoxamer 188, polyoxamer 407, polyoxyl castor oil such as polyoxyl 35 castor oil, polyoxyl hydrogenated castor oil such as polyoxyl 40 hydrogenated castor oil, propylene glycol diacetate, propylene glycol laurates such as propylene glycol dilaurate and propylene glycol monolaurate. Further examples include propylene glycol monopalmitostearate, quillaia, sorbitan esters, and sucrose esters.

Anionic surfactants suitable for the present disclosure include, for example, salts of perfluorocarboxylic acids and perfluorosulphonic acid, alkyl sulphate salts such as sodium dodecyl sulphate and ammonium lauryl sulphate, sulphate ethers such as sodium lauryl ether sulphate, and alkyl benzene sulphonate salts.

Cationic surfactants suitable for the present disclosure include, for example, quaternary ammonium compounds such as benzalkonium chloride, cetylpyridinium chlorides, benzethonium chlorides, and cetyl trimethylammonium bromides or other trimethylalkylammonium salts.

Zwitterionic surfactants include, but are limited to, for example dodecyl betaines, coca amphoglycinates and cocamidopropyl betaines.

In some embodiments of the present disclosure, the surfactant may comprise a phospholipid, derivative thereof, or analogue thereof. Such surfactants may, for example, be chosen from natural, synthetic, and semisynthetic phospholipids, derivatives thereof, and analogues thereof. Exemplary phospholipids surfactants include phosphatidylcholines with saturated, unsaturated and/or polyunsaturated lipids such as dioleoylphosphatidylcholine, dipentadecanoylphosphatidylcholine, dilauroylphosphatidylcholine, dimyristoylphosphatidylcholine, dipalmitoylphosphatidylcholine, distearoylphosphatidylcholine, di-eicopentaenoyl(EPA)choline, didocosahexaenoyl(DHA)choline, phosphatidylethanolamines, phosphatidylglycerols, phosphatidylserines and phosphatidylinositols. Other exemplary phospholipid surfactants include soybean lecithin, egg lecithin, diolelyl phosphatidylcholine, distearoyl phosphatidyl glycerol, PEG-ylated phospholipids, and dimyristoyl phosphatidylcholine.

Phospholipids may be “natural” or from a marine origin chosen from, e.g. phosphatidylcholine, phosphatidylethanolamine, phosphatidylserine and phosphatidylinosytol. The fatty acid moiety may be chosen from 14:0, 16:0, 16:1n-7, 18:0, 18:1n-9, 18:1n-7, 18:2n-6, 18:3n-3, 18:4n-3, 20:4n-6, 20:5n-3, 22:5n-3 and 22:6n-3, or any combinations thereof. In one embodiment, the fatty acid moiety is chosen from palmitic acid, EPA and DHA.

Other exemplary surfactants suitable for the present disclosure are listed in Table 7.

TABLE 7
Other surfactants

| Surfactant | Type | HBL Value |
|---|---|---|
| Ethylene glycol distearate | Nonionic | 1.5 |
| Glyceryl monostearate | Nonionic | 3.3 |
| Propylene glycol monostearate | Nonionic | 3.4 |
| Glyceryl monostearate | Nonionic | 3.8 |
| Diethylene glycol monolaurate | Nonionic | 6.1 |
| Acacia | Anionic | 8.0 |
| Cetrimonium bromide | Cationic | 23.3 |
| Cetylpyridinium chloride | Cationic | 26.0 |
| Polyoxamer 188 | Nonionic | 29.0 |
| Sodium lauryl sulphate | Anionic | 40 |

In some embodiments of the present disclosure, the at least one surfactant does not comprise Labrasol, Cremophor RH40, or the combination of Cremophor and Tween-80.

In some embodiments, the at least one surfactant has a hydrophilic-lipophilic balance (HLB) of less than about 10, such as less than about 9, or less than about 8.

Co-Surfactant

In some embodiments, compositions of the present disclosure further comprise at least one co-surfactant. As used herein the term “co-surfactant” means a substance added to, e.g., the preconcentrate in combination with the at least one surfactant to affect, e.g., increase or enhance, emulsification and/or stability of the preconcentrate, for example to aid in forming an emulsion. In some embodiments, the at least one co-surfactant is hydrophilic.

Examples of co-surfactants suitable for the present disclosure include, but are not limited to, short chain alcohols comprising from 1 to 6 carbons (e.g., ethanol), benzyl alcohol, alkane diols and triols (e.g., propylene glycol, glycerol, polyethylene glycols such as PEG and PEG 400), glycol ethers such as tetraglycol and glycofurol (e.g., tetrahydrofurfuryl PEG ether), pyrrolidine derivatives such as N-methylpyrrolidone (e.g., Pharmasolve®) and 2-pyrrolidone (e.g., Soluphor® P), and bile salts, for example sodium deoxycholate.

In some embodiments, the at least one co-surfactant comprises from about 1% to about 10%, by weight relative to the weight of the preconcentrate.

Solvent

In some embodiments, compositions of the present disclosure, such as the preconcentrate, further comprises at least one solvent. Hydrophilic solvents suitable for the present disclosure include, but are not limited to, alcohols, including water-miscible alcohols, such as absolute ethanol and/or glycerol, and glycols, for example glycols obtainable from an oxide such as ethylene oxide, such as 1,2-propylene glycol. Other non-limiting examples include polyols, such as polyalkylene glycol, e.g., poly(C₂₋₃)alkylene glycol such as polyethylene glycol.

In some embodiments of the present disclosure, the preconcentrate comprises at least one substance that acts both as a co-surfactant and a solvent, for example an alcohol such as ethanol. In other embodiments, the preconcentrate comprises at least one co-surfactant and at least one solvent that are different substances. For example, in some embodiments the preconcentrate comprises ethanol as the co-surfactant and glycerol as the solvent.

In some embodiments of the present disclosure, the preconcentrate is a pharmaceutical preconcentrate comprising a fatty acid oil mixture comprising at least 75% eicosapentaenoic acid (EPA) and docosahexaenoic acid (DHA), by weight of the fatty acid oil mixture, wherein the EPA and DHA are in a form chosen from ethyl ester and triglyceride; at least one free fatty acid; at least one surfactant; and at least one statin or pharmaceutically acceptable salt, hydrate, solvate, or complex thereof.

In one embodiment, the pharmaceutical preconcentrate comprises: a fatty acid oil mixture comprising at least 95% of EPA ethyl ester, DHA ethyl ester, or mixtures thereof, by weight of the fatty acid oil mixture; at least one free fatty acid chosen from linoleic, α-linolenic acid (ALA), γ-linoleic acid (GLA), and oleic acid; at least one surfactant chosen from polysorbate 20, polysorbate 80, and mixtures thereof; and at least one statin chosen from atorvastatin, simvastatin, rosuvastatin, and pharmaceutically acceptable salts, hydrates, solvates, and complexes thereof.

In another embodiment, the pharmaceutical preconcentrate comprises: a fatty acid oil mixture comprising from about 80% to about 88% EPA and DHA by weight of the fatty acid oil mixture, wherein the EPA and DHA are in ethyl ester form; at least one free fatty acid comprising oleic acid; at least one surfactant chosen from polysorbate 20, polysorbate 80, and mixtures thereof and at least one statin chosen from atorvastatin, simvastatin, rosuvastatin, and pharmaceutically acceptable salts, hydrates, solvates, and complexes thereof: wherein the at least one surfactant comprises less than 40%, by weight relative to the weight of the preconcentrate.

In another embodiment, the pharmaceutical preconcentrate comprises: a fatty acid oil mixture comprising from about 80% to about 88% EPA and DHA by weight of the fatty acid oil mixture, wherein the EPA and DHA are in ethyl ester form; at least one free fatty acid comprising linoleic acid; at least one Surfactant chosen from polysorbate 20, polysorbate 80, and mixtures thereof and at least one statin chosen from atorvastatin, simvastatin, rosuvastatin, and pharmaceutically acceptable salts, hydrates, solvates, and complexes thereof; wherein the at least one surfactant comprises less than 35%, by weight relative the weight of the preconcentrate.

In another embodiment, the pharmaceutical preconcentrate comprises: a fatty acid oil mixture comprising from about 80% to about 88% EPA and DHA by weight of the fatty acid oil mixture, wherein the EPA and DHA are in ethyl ester form; at least one free fatty acid comprising from about 80% to about 88% EPA and DHA, by weight of the at least one free fatty acid, wherein the EPA and DHA are in free acid form; at least one surfactant chosen from polysorbate 20, polysorbate 80, and mixtures thereof; and at least one statin chosen from atorvastatin, simvastatin, rosuvastatin, and pharmaceutically acceptable salts, hydrates, solvates, and complexes thereof. For example, the pharmaceutical preconcentrate may comprise K85EE as the fatty acid oil mixture, K85FA as the at least one free fatty acid, at least one surfactant chosen from polysorbate 20, polysorbate 80, and mixtures thereof; and atorvastatin calcium as the at least one statin or pharmaceutically acceptable salt, hydrate, solvate, or complex thereof.

In another embodiment, the pharmaceutical preconcentrate may comprise K85EE as the fatty acid oil mixture, K85FA as the at least one free fatty acid, at least one surfactant chosen from polysorbate 20 or polysorbate 80, and atorvastatin as the at least one statin or pharmaceutically acceptable salt, hydrate, solvate, or complex thereof, wherein the [K85EE]:[Tween]:[K85FA] ranges from e.g. about 5:2:0.5 to 5:4:2. In a further embodiment, the ratio between [K85EE]:[Tween]:[K85FA] is about [4-5]:[3-4]:[1-1.5].

In another embodiment, minimum of about 5-10% up to maximum of about 50% of fatty acid oil mixture comprising from about 80% to about 88% EPA and DHA by weight of the fatty acid oil mixture, wherein the EPA and DHA are in ethyl ester form, is substituted by a free fatty acid chosen from a K85-FA composition (corresponding to a K85-FA fatty acid profile achieved by hydrolyzing a K85-EE fatty acid ethyl ester composition) EPA, DPA, DHA, and combinations thereof. For example, the EPA-EE and DHA-EE content from 400 mg/g to 840 mg/g of total fatty acid oil mixture is replaced by 40 to 440 mg/g free fatty acid chosen from a K85-FA composition.

In some embodiments, the weight ratio of fatty acid oil mixture:surfactant of the preconcentrate ranges from about 1:1 to about 10:1, from about 1.1 to about 8:1, from 1:1 to about 7:1, from 1:1 to about 6:1, from 1:1 to about 5:1, from 1:1 to about 4:1, from 1:1 to about 3:1, or from 1:1 to about 2:1.

In some embodiments, the at least one surfactant comprises from about 5% to about 55%, by weight relative to the total weight of the preconcentrate. For example, in some embodiments, the at least one surfactant comprises from about 5% to about 35%, from about 10% to about 35%, from about 15% to about 35%, from about 15% to about 30%, or from about 20% to about 30%, by weight, relative to the total weight of the preconcentrate.

SNEDDS/SMEDDS/SEDDS

The preconcentrate of the present disclosure may be in a form of a self-nanoemulsifying drug delivery system (SNEDDS), a self-microemulsifying drug delivery system (SMEDDS), or a self emulsifying drug delivery system (SEDDS), wherein the preconcentrate forms an emulsion in an aqueous solution.

Without being bound by theory, it is believed that the preconcentrate forms a SNEDDS, SMEDDS, and/or SEDDS upon contact with gastric and/or intestinal media in the body, wherein the preconcentrate forms an emulsion comprising micelle particles. The emulsion may, for example, provide for increased or improved stability of the fatty acids for uptake in the body and/or provide increased or improved surface area for absorption. SNEDDS/SMEDDS/SEDDS may thus provide for enhanced or improved hydrolysis, solubility, bioavailability, absorption, or any combinations thereof of fatty acids in vivo.

Generally, known SNEDDS/SMEDDS/SEDDS formulations comprise ˜10 mg of a drug and ˜500 mg of surfactants/co-surfactants. The SNEDDS/SMEDDS/SEDDS presently disclosed may have the opposite relationship, i.e., the amount of API (e.g., the fatty acid oil mixture and the at least one statin or pharmaceutically acceptable salt, hydrate, solvate, or complex thereof) is greater than the amount of surfactant.

The SNEDDS/SMEDDS/SEDDS presently disclosed may comprise a particle size (i.e., particle diameter) ranging from about 5 am to about 10 μm. For example, in some embodiments, the particle size ranges from about 5 am to about 1 μm, such as from about 50 nm to about 750 nm, from about 100 nm to about 500 nm, from about 150 nm to about 350 nm.

Excipients

The compositions, preconcentrates, and/or SNEDDS/SMEDDS/SEDDS presently disclosed may further comprise at least one non-active pharmaceutical ingredient, e.g., excipient. Non-active ingredients may solubilise, suspend, thicken, dilute, emulsify, stabilize, preserve, protect, color, flavor, and/or fashion active ingredients into an applicable and efficacious preparation, such that it may be safe, convenient, and/or otherwise acceptable for use. Examples of excipients include, but are not limited to, carriers, fillers, extenders, binders, humectants, disintegrating agents (e.g., disintegrants and/or superdisintegrants), solution-retarding agents, absorption accelerators, wetting agents, absorbents, lubricants, coloring agents, buffering agents, chelating agents, dispersing agents, basic substances, and preservatives. Excipients may have more than one role or function, or may be classified in more than one group; classifications are descriptive only and are not intended to be limiting. In some embodiments, the excipient may be chosen from colloidal silicon dioxide, crospovidone, lactose monohydrate, lecithin, microcrystalline cellulose, polyvinyl alcohol, povidone, sodium lauryl sulfate, sodium stearyl fumarate, talc, titanium dioxide, and xanthum gum.

In some embodiments, the compositions, preconcentrates, and/or SNEDDS/SMEDDS/SEDDS presently disclosed further comprise at least one chelating agent. Examples of suitable chelating agents include, but are not limited to, aminopolycarboxylic acids such as EDTA and DTPA or pharmaceutically acceptable salts thereof including disodium EDTA and sodium calcium DTPA, and citric acid and pharmaceutically acceptable salts thereof. The at least one chelating agent may comprise from about 0.001% to about 10% by weight, such as from about 0.005% to about 5% by weight, or from about 0.01% to about 3% by weight.

In some embodiments, the compositions, preconcentrates, and/or SNEDDS/SMEDDS/SEDDS presently disclosed further comprise at least one basic substance. Examples of suitable basic substances include, but are not limited to, any pharmaceutically acceptable basic material such as L-arginine, benethamine, benzathine, basic calcium salts, choline, deanol, diethanolamine, diethylamine, 2-(diethylamino)-ethanol, ethanolamine, ethylenediamine. N-methyl-glucamine, hydravamine, 1H-imidazole, L-lysine, basic magnesium salts, 4-(2-hydroxyethyl)-morpholine, piperazine, basic potassium salts, 1-(2-hydroxyethyl)-pyrrolidine, basic sodium salts, triethanolamine, tromethamine, basic zinc salts, and other organic pharmaceutically acceptable bases.

In some embodiments, the compositions, preconcentrates, and/or SNEDDS/SMEDDS/SEDDS presently disclosed further comprise at least one buffering agent. Examples of suitable basic substances include, but are not limited to, any pharmaceutically acceptable buffering material such as pharmaceutically acceptable salts of inorganic acids, salts of organic acids, and salts of organic bases. Examples of salts of pharmaceutically acceptable inorganic acids include salts with phosphoric acid such as sodium or potassium phosphate or hydrogen phosphate, dibasic sodium phosphate, sodium, potassium, magnesium or calcium carbonate or hydrogen carbonate, sulphate, or mixtures thereof. Examples for salts of organic acids include potassium or sodium salts of acetic acid, citric acid, lactic acid, ascorbic acid, fatty acids like for eample EPA/DHA salts, maleic acid, benzoic acid, lauryl sulphuric acid.

The compositions, preconcentrates, and/or SNEDDS/SMEDDS/SEDDS presently disclosed may further comprise at least one antioxidant. Examples of antioxidants suitable for the present disclosure include, but are not limited to, α-tocopherol (vitamin E), calcium disodium EDTA, alpha tocoferyl acetates, butylhydroxytoluenes (BHT), and butylhydroxyanisoles (BHA). Other examples of antioxidants include ascorbic acid and pharmaceutically acceptable salts thereof such as sodium ascorbate, pharmaceutically acceptable esters of ascorbic acid including fatty acid ester conjugates, propyl gallate, citric acid and pharmaceutically acceptable salts thereof, malic acid and pharmaceutically acceptable salts thereof, and sulfite salts such as sodium sulfite and mixtures thereof.

The compositions, preconcentrates, and/or SNEDDS/SMEDDS/SEDDS presently disclosed may comprise from about 0.001% to about 10% by weight of at least one antioxidant with respect to the total weight of the composition and/or preconcentrate, such as from about 0.005% to about 5% by weight, or from about 0.01% to about 3% by weight.

In some embodiments, the compositions, preconcentrates, and/or SNEDDS/SMEDDS/SEDDS presently disclosed further comprise at least one antioxidant and at least one excipient. In one embodiment, for example, the compositions, preconcentrates, and/or SNEDDS/SMEDDS/SEDDS comprise a mixture of at least three compounds chosen from antioxidants, basic substances, chelating agents, and buffering agents. In one embodiment, the compositions, preconcentrates, and/or SNEDDS/SMEDDS/SEDDS comprise at least one antioxidant and at least one excipient chosen from chelating agents, bufferent agents, and basic materials. In one embodiment, the compositions, preconcentrates, and/or SNEDDS/SMEDDS/SEDDS comprise at least one chelating agent, at least one basic material, and at least one buffering agent. In another embodiment, the compositions, preconcentrates, and/or SNEDDS/SMEDDS/SEDDS comprise at least one chelating agent and at least one basic material. In yet another embodiment, the compositions, preconcentrates, and/or SNEDDS/SMEDDS/SEDDS comprise at least one chelating agent and at least one buffering agent. All of the aforementioned compositions and/or preconcentrates may be sufficiently stable for pharmaceutical use. For example, the compositions, preconcentrates, and/or SNEDDS/SMEDDS/SEDDS presently disclosed may have a shelf-life of at least 2 years, e.g., no more than 2% degradation of statin and no more than 5% degradation of EPA/DHA ethyl ester over a period of 12 months according to ICH (International Conference on Harmonization) Guidelines (i.e., temperature, humidity).

In further embodiments of the present disclosure, the pharmaceutical preconcentrate comprises a fatty acid oil mixture, at least one surfactant chosen from Tween-20 and Tween-80, at least one fatty acid, at least one antioxidant; and at least one statin or pharmaceutically acceptable salt, hydrate, solvate, or complex thereof. For example, the fatty acid oil mixture is present in an amount ranging from about 45% to about 70% by weight, such as from about 45% to about 55% by weight, relative to the weight of the preconcentrate and/or composition; the at least one surfactant is present in an amount ranging from about 5% to about 55% by weight, such as from about 10% to about 30%, such as from about 10% to about 25%, such as about 20% by weight, relative to the weight of the preconcentrate and/or composition; the at least one fatty acid is present in an amount ranging from about 5% to about 20% by weight, such as from about 10% to about 15% by weight, relative to the weight of the preconcentrate and/or composition; and the at least one antioxidant is present in an amount ranging from about 0.001% to about 10% by weight, such as from about 0.005% to about 5%, such as from about (101% to about 3% by weight, relative to the weight of the preconcentrate and/or composition. Further for example, the pharmaceutical preconcentrate comprises about 50% K85-EE; about 38% Tween-20, about 13% oleic acid, about 0.03% BHA, and at least one statin or pharmaceutically acceptable salt, hydrate, solvate, or complex thereof.

In other embodiments of the present disclosure, the pharmaceutical preconcentrate comprises a fatty acid oil mixture, at least one surfactant chosen from Tween-20 and Tween-80, at least one fatty acid; and at least one statin or pharmaceutically acceptable salt, hydrate, solvate, or complex thereof. For example, the fatty acid oil mixture is present in an amount ranging from about 45% to about 70% by weight, such as from about 45% to about 55% by weight, relative to the weight of the preconcentrate and/or composition; the at least one surfactant is present in an amount ranging from about 5% to about 55% by weight, such as from about 10% to about 30%, such as from about 10% to about 25%, such as about 20% by weight, relative to the weight of the preconcentrate and/or composition; and the at least one fatty add is present in an amount ranging from about 5% to about 20% by weight, such as from about 10% to about 15% by weight, relative to the weight of the preconcentrate and/or composition. Further for example, the pharmaceutical preconcentrate comprises about 400 mg K85-EE, about 300 mg Tween-20, about 100 mg K85-FA; and at least one statin or pharmaceutically acceptable salt, hydrate, solvate, or complex thereof.

### Formulations

The compositions and/or preconcentrates presently disclosed may be administered, e.g., in capsule, caplet, tablet, or any other forms suitable for drug delivery.

In some embodiments, for example, the compositions and/or preconcentrates are loaded into a tablet. When the dosage form is in the form of tablets, the tablets may be, for example, disintegrating tablets, fast dissolving tablets, effervescent tablets, fast melt tablets, and/or mini-tablets. Tablet formulations are described, for example, in patent publication WO 2006/000229.

Further, the dosage form can be of any shape suitable for oral administration, such as spherical, oval, ellipsoidal, cube-shaped, regular, and/or irregular shaped. The dosage forms can be prepared according to processes known in the art and can include one or more additional pharmaceutically-acceptable excipients as discussed above.

The compositions and/or preconcentrates presently disclosed may be encapsulated, such as a gelatin capsule. In some embodiments, the compositions and/or preconcentrates presently disclosed comprise microcapsules encapsulated with a material chosen from cyclodextrin, and gelatin. Examples of cyclodextrins include, but are not limited to, substituted and unsubstituted cyclodextrins, e.g., alpha-cyclodextrin, beta-cyclodextrin, gamma-cyclodextrin, alkylated cyclodextrins such as methylated cyclodextrins and 2-hydroxypropyl-cyclodextrins. In at least one embodiment, the compositions and/or preconcentrates are polymer-free.

In one embodiment, the composition and/or preconcentrate comprises two compartments wherein a first compartment comprises at least a first API (e.g., fatty acid oil mixture), and a second compartment comprises at least a second API (e.g., statin or pharmaceutically acceptable salt, hydrate, solvate, or complex thereof). In one embodiment, the first API comprises a fatty acid oil mixture comprising EPA and DHA, and the second API comprises atorvastatin calcium. For example, the composition presently disclosed may comprise a two compartment capsule, wherein a first compartment comprises a fatty acid oil mixture and at least one free fatty acid, and a second compartment comprises at least one statin or pharmaceutically acceptable salt, hydrate, solvate, or complex thereof. Further, for example, the preconcentrate presently disclosed may comprise a two compartment capsule, wherein a first compartment comprises a fatty acid oil mixture, at least one free fatty acid, and at least one surfactant; and a second compartment comprises at least one statin or pharmaceutically acceptable salt, hydrate, solvate, or complex thereof.

The two compartment capsule may comprise two compartments adjacent to each other, or may comprise one compartment inside a second compartment. Examples of two compartment capsules include, but are not limited to, a DuoCap™ capsule delivery system (Encap Drug Delivery).

The DuoCap™ is a single oral dosage unit that comprises a capsule-in-a-capsule. The inner and outer capsules may contain the same active agent providing multiple release profiles from the dosage unit, for example the outer capsule comprises an immediate release formulation and the inner capsule comprises a controlled release formulation. In addition to modifying the release profiles, it is also possible to formulate the inner and outer capsules to target release at different areas of the GI tract (small intestine or colon). Alternatively, the two compartment capsule may comprise different active agents for use in combination therapies, or for actives that may be incompatible in a single capsule.

In one embodiment of the present disclosure, the capsule comprises an inner compartment (e.g., inner capsule) comprising a fatty acid oil mixture and an outer compartment (e.g., outer capsule) comprising at least one statin or pharmaceutically acceptable salt, hydrate, solvate, or complex thereof. For example, the capsule may comprise an inner capsule comprising a fatty acid oil mixture and at least one free fatty acid, and an outer capsule comprising at least one statin chosen from atorvastatin, rosuvastatin, simvastatin, and a pharmaceutically acceptable salt, hydrate, solvate, or complex thereof. In another embodiment, the capsule comprises an inner capsule comprising a fatty acid oil mixture, at least one free fatty acid, and at least one surfactant; and an outer capsule comprising at least one statin chosen from atorvastatin, rosuvastatin, simvastatin, and a pharmaceutically acceptable salt, hydrate, solvate, or complex thereof. In other embodiments, the capsule comprises an inner capsule comprising at least one statin chosen from atorvastain, rosuvastatin, simvastatin, and a pharmaceutically acceptable salt, hydrate, solvate, or complex thereof; and the outer capsule comprises a fatty acid mixture. In some embodiments, the compartment comprising the fatty acid oil mixture is formulated in a form chosen from liquid, semi-solid, powder and pellet form. Moreover, the two compartment capsule can further be coated with at least one enteric coating or with Encap's colonic delivery system, ENCODE™.

In some embodiments, the at least one statin or pharmaceutically acceptable salt, hydrate, solvate, or complex thereof is dissolved in the fatty acid oil mixture with no crystal formation of statin before administration. In other embodiments, the compositions and/or preconcentrates comprise an emulsion or suspension, such as a nanoemulsion or a microemulsion, wherein the at least one statin or pharmaceutically acceptable salt, hydrate, solvate, or complex thereof is suspended in the fatty acid oil mixture with little to no statin dissolved in the oil.

Further, in some embodiments, the compositions and/or preconcentrates comprise an emulsion comprising microcapsules of at least one statin or pharmaceutically acceptable salt, hydrate, solvate, or complex thereof. For example, in at least one embodiment of the present disclosure, the composition comprises statin microcapsules suspended in a combination of a fatty acid oil mixture and at least one free fatty acid. In another embodiment, the preconcentrate comprises statin microcapsules suspended in a combination of a fatty acid oil mixture, at least one free fatty acid, and at least one surfactant. The statin microcapsules may be encapsulated, for example, in a material chosen from cyclodextrin and alginate. The compositions and/or preconcentrates comprising the statin microcapsules may be encapsulated in a material that may be the same or different from that of the statin microcapsules. For example, in some embodiments, the compositions and/or preconcentrates comprise gelatin capsules that comprise statin microcapsules, wherein the at least one statin is encapsulated in a material chosen from cyclodextrin and alginate.

In other embodiments, the compositions and/or preconcentrates comprise an encapsulated fatty acid oil mixture wherein the capsule shell wall, such as a gelatin shell, comprises at least one statin or pharmaceutically acceptable salt, hydrate, solvate, or complex thereof, such as atorvastatin, rosuvastatin, or simvastatin. The statin may be added to the encapsulation material during preparation of the capsule shell, or may also be spray-dried onto the outside of a prepared capsule shell.

The present disclosure also provides for one or more enteric coating layer(s) formed from gastro-resistant materials, such as pH-dependent and/or pH-independent polymers. Coatings with pH-independent profiles generally erode or dissolve away after a predetermined period, and the period is generally directly proportional to the thickness of the coating. Coatings with pH-dependent profiles, on the other hand, can generally maintain their integrity while in the acid pH of the stomach, but erode or dissolve upon entering the more basic upper intestine. Such coatings generally serve the purpose of delaying the release of a drug for a predetermined period. For example, such coatings can allow the dosage form to pass through the stomach without being substantially subjected to stomach acid or digestive juices for delayed release outside of the stomach.

Examples of enteric coating materials include, but are not limited to, acrylic and cellulosic polymers and copolymers, e.g., methacrylic acid, copolymers between methacrylic acid and methyl methacrylate or methyl acrylate, copolymers between metacrylic acid and ethyl methacrylate or ethyl acrylate, polysaccharides like cellulose acetate phthalate, hydroxypropyl methyl cellulose phthalate, hydroxypropyl methyl cellulose acetate succinate, and polyvinyl acetate phthalate. Additional useful enteric coating materials include pharmaceutically acceptable acidic compounds that may not dissolve at the low pH in the stomach, but at higher pH in the lower part of the gastrointestinal system.

The enteric coating material may comprise one or more plasticizer(s) to improve the mechanical properties of pH-sensitive material(s). Typical plasticizers include triethyl citrate, triacetin, polyethylene glycols, propylene glycol, phthalates, sorbitol and glycerin. The amount of plasticizer suitable for enteric coating according to the present disclosure may vary depending upon the chemical composition of the enteric coating, the chemical nature of the encapsulating material(s), and the size and the shape of the capsules. In some embodiments, for example, the plasticizer for capsules comprising EPA and DHA ethyl esters comprises from about 10% to about 60% by weight of the enteric coating material.

In some embodiments, the compositions and/or preconcentrates comprise one or more sub-layer(s) between the capsule shell and an enteric coating and/or one or more top-layer(s) and/or top-layer(s) over the enteric coating. The chemical composition of sub-layers and top-layers may vary depending upon the overall composition of the capsule. Typical sub-layers and top-layers comprise one or more film-forming agent(s) such as polysaccharides, e.g., hydroxypropyl methyl cellulose.

In some embodiments of the present disclosure, the capsule fill content ranges from about 0.400 g to about 1.600 g. For example, in some embodiments, the capsule fill content ranges from about 0.400 g to about 1.300 g, from about 0.600 g to about 1.200 g, from about 0.600 g to about 0.800 g, from about 0.800 g to about 1.000, from about 1.000 g to about 1.200 g, or any amount in between. For example, in some embodiments the capsule fill content is about 0,600 g, about 0.800 g, about 1.000 g, or about 1.200 g.

The capsules presently disclosed may be manufactured in low oxygen conditions to inhibit oxidation during the manufacturing process. Preparation of capsules and/or microcapsules in accordance with the present disclosure may be carried out following any of the methods described in the literature. Examples of such methods include, but are not limited to, simple coacervation methods (see, e.g., ES 2009346, EP 0052510, and EP 0346879), complex coacervation methods (see, e.g., GB 1393805), double emulsion methods (see, e.g., U.S. Pat. No. 4,652,441), simple emulsion methods (see, e.g., U.S. Pat. No. 5,445,832), and solvent evaporation methods (see, e.g., GB 2209937). Those methods may, for example, provide for continuous processing and flexibility of batch size.

in other embodiments, the compositions and/or preconcentrates are loaded into a tablet, wherein the tablet is coated by at least one of a film coating, a sub-layer, and an enteric coating. Suitable sub-layer and enteric coating materials are described above. Suitable coating materials for the film coating include, for example, methylcellulose, hydroxypropylmethylcellulose, hydroxypropylcellulose, acrylic polymers, ethylcellulose, cellulose acetate phthalate, polyvinyl acetate phthalate, hydroxypropyl methylcellulose phthalate, polyvinylalcohol, sodium carboxymethylcellulose, cellulose acetate, cellulose acetate phthalate, gelatin, methacrylic acid copolymer, polyethylene glycol, shellac, sucrose, titanium dioxide, carnauba wax, microcrystalline wax, and zein.

Methods or Uses

The present disclosure further encompasses methods of treating at least one health problem in a subject in need thereof. The compositions presently disclosed may be administered, e.g., in capsule, caplet, tablet or any other drug delivery forms, such as the formulations described above, to a subject for therapeutic treatment of at least one health problem including, for example, irregular plasma lipid levels, cardiovascular functions, immune functions, visual functions, insulin action, neuronal development, heart failure, and post myocardial infarction. In some embodiments, the at least one health problem is chosen from mixed dyslipidemia, dyslipidemia, hypertriglyceridemia, and hypercholesterolemia.

The present disclosure further provides for a method for treating at least one health problem while enhancing at least one parameter chosen from hydrolysis, solubility, bioavailability, absorption, and combinations thereof of eicosapentaenoic acid (EPA) and docosahexaenoic acid (DHA) comprising combining: a fatty acid oil mixture comprising EPA and DHA in a form chosen from ethyl ester and triglyceride, at least one free fatty acid, and at least one statin or pharmaceutically acceptable salt, hydrate, solvate, or complex thereof. In some embodiments, the method comprises combining: a fatty acid oil mixture comprising EPA and DHA in a form chosen from ethyl ester and triglyceride; at least one free fatty acid; at least one surfactant; and at least one statin or pharmaceutically acceptable salt, hydrate, solvate, or complex thereof; wherein the fatty acid oil mixture, the at least one free fatty acid, the at least one surfactant, and the at least one statin or pharmaceutically acceptable salt, hydrate, solvate, or complex thereof form a preconcentrate. In addition, the preconcentrate can form a self-nanoemulsifying drug delivery system (SNEDDS), self-microemulsifying drug delivery system (SMEDDS), or self-emulsifying drug delivery system (SEDDS) in an aqueous solution. The bioavailablity may be increased.

In some embodiments, the composition further comprises at least one surfactant to form a preconcentrate for administration to a subject in need thereof to treat at least one health problem. In some embodiments, the preconcentrate forms a self-nanoemulsifying drug delivery system (SNEDDS), a self-microemulsifying drug delivery system (SMEDDS), or a self-emulsifying drug delivery system (SEDDS) in an aqueous solution. In some embodiments, the aqueous solution is gastric media and/or intestinal media.

The total daily dosage of the fatty acid oil mixture may range from about 0.600 g to about 6.000 g. For example, in some embodiments, the total dosage of the fatty acid oil mixture ranges from about 0.800 g to about 4.000 g, from about 1.000 g to about 4.000 g, or from about 1,000 g to about 2.000 g. In one embodiment, the fatty acid oil mixture is chosen from K85EE and AGP 103 fatty acid oil compositions.

The composition and/or preconcentrates presently disclosed may be administered in from 1 to 10 dosages, such as from 1 to 4 times a day, such as once, twice, three times, or four times per day, and further for example, once, twice or three times per day. The administration may be oral or any other form of administration that provides a dosage of fatty acids, e.g., omega-3 fatty acids, and at least one statin or pharmaceutically acceptable salt, hydrate, solvate, or complex thereof to a subject.

The following examples are intended to illustrate the present disclosure without, however, being limiting in nature. It is understood that the skilled artisan will envision additional embodiments consistent with the disclosure provided herein.

### EXAMPLES

### Example 1

### Preconcentrates

Different preconcentrates comprising a fatty acid oil mixture, a free fatty acid, and a surfactant were prepared as described in Table 9. To prepare the preconcentrates, the components were mixed according to the schemes identified below on a weight to weight basis. The preconcentrates were visually inspected after mixing and again after being stored for 24 hours at room temperature. Under the Preconcentrate heading, a “clear” designation represents a transparent homogenous mixture; an “unclear” designation represents a nonhomogenous mixture, where some turbidity can be observed by visual inspection. The degree of turbidity was not determined.

All clear preconcentrates were emulsified in gastric media, by adding gastric media (2 ml) to approximately 100 mg of the preconcentrate. The composition of the gastric media is shown in Table 8.

TABLE 8
Composition of Gastric Media.
Gastric Media

| | |
|---|---|
| Bile salts, Porcine (mM) | 0.08 |
| Lechitin(mM) | 0.02 |
| Sodium chloride (mM) | 34.2 |
| Pepsin (mg/ml) | 0.1 |
| pH | 1.6 (adjust with 1M HCl) |
| Osmolarity(mOsm/kg) | 120 |

The outcome of the emulsification was recorded approximately 3 hours after mixing. A majority of the preconcentrates formed milky emulsions immediately after mixing. Emulsions that stayed milky and homogenous after 3 hours are described as “milky,” under the Emulsion heading. Emulsions that separated or became nonhomogenous or where oil drops were observed are described as “separates,” under the Emulsion heading.

Selected emulsions were further characterized by determining the particle size. Particle size was measured using a Malvern Zetasizer (Malvern Instrument, Worcestershire, UK) with particle size measuring range of 0, 5-6000 nm and Zeta potential of particle range of 3 nm-10 μm. The particle size was measured in triplicate. The K85EE (EE=ethyl ester) fatty acid composition used herein is sold in a gelatin capsule and branded primarily under the trademarks Lovaza™ or Omacor®.

TABLE 9
Preconcentrates.

| | K85- | Tween- | | Total | | | | Particle |
|---|---|---|---|---|---|---|---|---|
| | EE | 20 | Oleic Acid | vol. | | Pre- | | Size |
| No. | (mg) | (mg) | (mg) | (mg) | Ratio | conc. | Emulsion | (nm) |
| 1 | 451.4 | 234.3 | 99 | 784.7 | 57:29:12 | Unclear | — | — |
| 2 | 448.8 | 299.7 | 53.8 | 802.3 | 55:37:6 | Unclear | — | — |
| 3 | 451.2 | 324.7 | 24.7 | 800.6 | 56:40:3 | Unclear | — | — |
| 10 | 400 | 300 | 100 | 800 | 50:37:12 | Clear | Milky | 271 |
| 11 | 404 | 298 | 97 | 799 | 50:37:12 | Clear | Milky | — |
| 12 | 500 | 300 | 217 | 1017 | 49:29:21 | Clear | Separates | — |
| 13 | 398 | 300 | 99 | 797 | 49:37:12 | Clear | Milky | 257 |
| 14 | 399 | 252 | 98 | 749 | 53:33:13 | Clear | Separates | 226 |
| 15 | 400 | 204 | 102 | 706 | 56:28:14 | Clear | Separates | 199 |
| 21 | 450 | 198 | 133 | 781 | 57:25:17 | Clear | Separates | — |
| 23 | 549 | 204 | 169 | 922 | 59:22:18 | Clear | Separates | — |
| 24 | 600 | 200 | 178 | 978 | 61:20:18 | Clear | Separates | — |
| 26 | 453 | 214 | 121 | 788 | 57:27:15 | Clear | Separates | — |
| 27 | 456 | 220 | 121 | 797 | 57:27:15 | Clear | Separates | — |
| 28 | 452 | 228 | 144 | 824 | 54:27:17 | Clear | Separates | — |
| 29 | 448 | 230 | 122 | 800 | 56:28:15 | Clear | Separates | — |
| 30 | 452 | 242 | 124 | 818 | 55:29:15 | Clear | Separates | — |
| 31 | 449 | 251 | 124 | 824 | 54:30:15 | Clear | Milky | — |
| 32 | 448 | 260 | 123 | 831 | 53:31:14 | Clear | Separates | — |
| 33 | 452 | 270 | 121 | 843 | 53:32:14 | Clear | Separates | — |
| 34 | 449 | 281 | 123 | 853 | 52:32:14 | Clear | Separates | — |
| 35 | 448 | 290 | 121 | 859 | 52:33:14 | Clear | Separates | — |
| | K85- | | | Total | | | | Particle |
| | EE | Tween- | Ricinoleic | Vol. | | Pre- | | Size |
| No. | (mg) | 20 (mg) | Acid (mg) | (mg) | Ratio | conc. | Emulsion | (nm) |
| 36 | 402 | 298 | 98 | 798 | 50:37:12 | Clear | Milky | 277 |
| 37 | 402 | 250 | 100 | 752 | 53:33:13 | Clear | Milky | 268 |
| 38 | 400 | 200 | 100 | 700 | 57:28:14 | Unclear | — | — |
| 39 | 450 | 250 | 100 | 800 | 56:31:12 | Clear | Milky | — |
| 43 | 400 | 110 | 100 | 610 | 65:18:16 | Clear | Separates | — |
| 44 | 500 | 270 | 105 | 875 | 57:30:12 | Clear | Separates | — |
| 45 | 505 | 295 | 103 | 903 | 55:32:11 | Clear | Milky | — |
| 46 | 525 | 250 | 143 | 918 | 57:27:15 | Clear | Separates | — |
| 47 | 500 | 252 | 118 | 870 | 57:28:13 | Clear | Separates | — |
| 48 | 297 | 293 | 145 | 735 | 40:39:19 | Clear | Separates | — |
| 49 | 500 | 260 | 127 | 887 | 56:29:14 | Clear | Separates | — |
| 50 | 499 | 285 | 106 | 890 | 56:32:11 | Clear | Separates | — |
| 51 | 403 | 298 | 193 | 894 | 45:33:21 | Clear | Milky | — |
| 52 | 460 | 250 | 90 | 800 | 57:31:11 | Clear | — | — |
| | K85- | Tween- | Ricinoleic | Total | | | | Particle |
| | EE | 40 | acid | Vol. | | | | Size |
| No. | (mg) | (mg) | (mg) | (mg) | Ratio | Pre-conc. | Emulsion | (nm) |
| 53 | 450 | 255 | 98 | 803 | 56:31:12 | Clear | Milky | 237 |
| 55 | 498 | 220 | 98 | 816 | 61:26:12 | Clear | Milky | 226 |
| 56 | 505 | 202 | 106 | 813 | 62:24:13 | Clear | Separates | — |
| 57 | 500 | 200 | 100 | 800 | 62:25:12 | Clear | Separates | — |
| 58 | 552 | 152 | 102 | 806 | 68:18:12 | Clear | Separates | — |
| | K85- | Tween- | Ricinoleic | Total | | | | Particle |
| | EE | 60 | Acid | Vol. | | | | Size |
| No. | (mg) | (mg) | (mg) | (mg) | Ratio | Pre-conc. | Emulsion | (nm) |
| 70 | 500 | 200 | 100 | 800 | 62:25:12 | Clear | Milky | — |
| 71 | 500 | 150 | 100 | 750 | 66:20:13 | Clear | Separates | — |
| 72 | 529 | 180 | 104 | 813 | 65:22:12 | Clear | Separates | — |
| 73 | 518 | 200 | 102 | 820 | 63:24:12 | Clear | Separates | — |
| | K85- | Tween- | Ricinoleic | Total | | | | Particle |
| | EE | 80 | Acid | Vol. | | | | Size |
| No. | (mg) | (mg) | (mg) | (mg) | Ratio | Pre-conc. | Emulsion | (nm) |
| 54 | 450 | 270 | 105 | 825 | 54:32:12 | Clear | Separates | — |

| | K85- | Cremophor | Ricinoleic | Total | | | | Particle |
|---|---|---|---|---|---|---|---|---|
| | EE | EL | Acid | Vol. | | | | Size |
| No. | (mg) | (mg) | (mg) | (mg) | Ratio | Pre-conc. | Emulsion | (nm) |
| 40 | 399.9 | 300 | 106.4 | 806.3 | 49:37:13 | Unclear | — | — |
| 41 | 400 | 256.9 | 137 | 793.9 | 50:32:17 | Unclear | — | — |

| | K85- | | Ricinoleic | Total | | | | Particle |
|---|---|---|---|---|---|---|---|---|
| | EE | Soritol | Acid | Vol. | | Pre- | | Size |
| No. | (mg) | (mg) | (mg) | (mg) | Ratio | conc. | Emulsion | (nm) |
| 42 | 400 | 211 | 104 | 715 | 55:29:14 | Clear/solid | — | — |
| | | | | | | when | | |
| | | | | | | cooled | | |
| | K85- | PEG- | Ricinoleic | Total | | | | Particle |
| | EE | 400 | Acid | Vol. | | | | Size |
| No. | (mg) | (mg) | (mg) | (mg) | Ratio | Pre-conc. | Emulsion | (nm) |
| 16 | 399.9 | 310.2 | 162.6 | 872.7 | 45:35:18 | Clear | Separates | — |
| 17 | 398.3 | 256.8 | 157.9 | 813 | 48:31:19 | Clear | Separates | — |
| 18 | 402.4 | 198.7 | 147.5 | 748.6 | 53:26:19 | Clear | Separates | — |
| | K85- | Tween- | | Total | | | | Particle |
| | EE | 20 | PEG-400 | Vol. | | | | Size |
| No. | (mg) | (mg) | (mg) | (mg) | Ratio | Pre-conc. | Emulsion | (nm) |
| 19 | 398.2 | 297.9 | 214.7 | 910.8 | 43:32:23 | Unclear | — | — |
| 20 | 403 | 248.2 | 145.3 | 796.5 | 50:31:18 | Unclear | — | — |
| | K85- | | α-Linoleic | Total | | | | Particle |
| | EE | Tween- | Acid | Vol. | | | | Size |
| No. | (mg) | 20 (mg) | (mg) | (mg) | Ratio | Pre-conc. | Emulsion | (nm) |
| 74 | 402 | 300 | 100 | 802 | 50:37:12 | Clear | Milky | — |
| 75 | 454 | 249 | 98 | 801 | 56:31:12 | Slightly | Separates | — |
| | | | | | | dense | | |
| 76 | 502 | 204 | 103 | 809 | 62:25:12 | Slightly | Separates | — |
| | | | | | | dense | | |
| | K85- | Tween- | α-Linoleic | Total | | | | Particle |
| | EE | 40 | Acid | Vol. | | | | Size |
| No. | (mg) | (mg) | (mg) | (mg) | Ratio | Pre-conc. | Emulsion | (nm) |
| 77 | 403 | 299 | 108 | 810 | 49:36:13 | Clear/ | Separates | — |
| | | | | | | Precipitate | | |
| 78 | 456 | 252 | 110 | 818 | 55:30:13 | Clear/ | Separates | — |
| | | | | | | Precipitate | | |
| 79 | 503 | 217 | 103 | 823 | 61:26:12 | Clear/ | Separates | — |
| | | | | | | Precipitate | | |
| | K85- | Tween- | α-Linoleic | Total | | | | Particle |
| | EE | 60 | Acid | Vol. | | | | Size |
| No. | (mg) | (mg) | (mg) | (mg) | Ratio | Pre-conc. | Emulsion | (nm) |
| 80 | 402 | 313 | 104 | 819 | 49:38:12 | Clear | Separates | — |
| 81 | 459 | 205 | 100 | 764 | 60:26:13 | Clear | Separates | — |
| 82 | 498 | 198 | 106 | 802 | 62:24:13 | Clear | Separates | — |
| | K85- | Tween- | α-Linoleic | Total | | | | Particle |
| | EE | 80 | Acid | Vol. | | | | Size |
| No. | (mg) | (mg) | (mg) | (mg) | Ratio | Pre-conc. | Emulsion | (nm) |
| 83 | 407 | 317 | 102 | 826 | 49:38:12 | Clear | Milky | 261.3 |
| 84 | 455 | 256 | 110 | 821 | 55:31:13 | Clear | Milky | 260.8 |
| 85 | 498 | 208 | 102 | 808 | 61:25:12 | Clear | Milky | 274.5 |
| | K85- | Tween- | Erucuc | Total | | | | Particle |
| | EE | 20 | Acid | Vol. | | | | Size |
| No. | (mg) | (mg) | (mg) | (mg) | Ratio | Pre-conc. | Emulsion | (nm) |
| 86 | 401 | 300 | 99 | 800 | 50:37:12 | Clear | Semi | — |
| | | | | | | | Milky | |
| 87 | 451 | 250 | 105 | 806 | 55:31:13 | Clear | Separates | — |
| 88 | 504 | 204 | 102 | 810 | 62:25:12 | Clear | Separates | — |
| | K85- | Tween- | Erucuc | Total | | | | Particle |
| | EE | 40 | Acid | Vol. | | | | Size |
| No. | (mg) | (mg) | (mg) | (mg) | Ratio | Pre-conc. | Emulsion | (nm) |
| 89 | 401 | 298 | 102 | 801 | 50:37:12 | Clear | Separates | — |
| 90 | 451 | 254 | 99 | 804 | 56:31:12 | Clear | Separates | — |
| 91 | 504 | 219 | 103 | 826 | 61:26:12 | Clear | Separates | — |
| | K85- | Tween- | Erucuc | Total | | | | Particle |
| | EE | 60 | Acid | Vol. | | | | Size |
| No. | (mg) | (mg) | (mg) | (mg) | Ratio | Pre-conc. | Emulsion | (nm) |
| 92 | 401 | 301 | 104 | 806 | 49:37:12 | Clear | Separates | — |
| 93 | 454 | 267 | 101 | 822 | 55:32:12 | Clear | Separates | — |
| 94 | 497 | 202 | 100 | 799 | 62:25:12 | Clear | Separates | — |
| | K85- | Tween- | Erucuc | Total | | | | Particle |
| | EE | 60 | Acid | Vol. | | | | Size |
| No. | (mg) | (mg) | (mg) | (mg) | Ratio | Pre-conc. | Emulsion | (nm) |
| 95 | 406 | 298 | 100 | 804 | 50:37:12 | Clear | Separates | — |
| 96 | 450 | 251 | 102 | 803 | 56:31:12 | Clear | Separates | — |
| 97 | 502 | 205 | 122 | 829 | 60:24:14 | Clear | Separates | — |
| | | | α- | | | | | |
| | K85- | Tween- | Linolenic | Total | | | | Particle |
| | EE | 20 | acid | Vol. | | | | Size |
| No. | (mg) | (mg) | (mg) | (mg) | Ratio | Pre-conc. | Emulsion | (nm) |
| 98 | 401 | 308 | 105 | 814 | 49:37:12 | Clear | Milky, | — |
| | | | | | | | beginning | |
| | | | | | | | separation | |
| 102 | 450 | 264 | 108 | 822 | 54:32:13 | Clear | Milky, | — |
| | | | | | | | beginning | |
| | | | | | | | separation | |
| 106 | 501 | 200 | 111 | 812 | 61:24:13 | Clear | Milky, with | — |
| | | | | | | | separation | |
| | | | α- | | | | | |
| | K85- | Tween- | Linolenic | Total | | | | Particle |
| | EE | 40 | Acid | Vol. | | | | Size |
| No. | (mg) | (mg) | (mg) | (mg) | Ratio | Pre-conc. | Emulsion | (nm) |
| 99 | 402 | 302 | 102 | 806 | 49:37:12 | Clear | Milky, | — |
| | | | | | | | beginning | |
| | | | | | | | separation | |
| 103 | 452 | 254 | 101 | 807 | 56:31:12 | Clear | Milky, with | — |
| | | | | | | | separation | |
| 107 | 502 | 206 | 108 | 816 | 61:25:13 | Clear | Milky, with | — |
| | | | | | | | separation | |
| | | | α- | | | | | |
| | K85- | Tween- | Linolenic | Total | | | | Particle |
| | EE | 60 | Acid | Vol. | | | | Size |
| No. | (mg) | (mg) | (mg) | (mg) | Ratio | Pre-conc. | Emulsion | (nm) |
| 100 | 403 | 303 | 103 | 809 | 49:37:12 | Clear | Milky, | — |
| | | | | | | | beginning | |
| | | | | | | | separation | |
| 104 | 450 | 249 | 102 | 801 | 56:31:12 | Clear | Milky, with | — |
| | | | | | | | separation | |
| 108 | 506 | 200 | 100 | 806 | 62:24:12 | Unclear | Milky, | — |
| | | | | | | | beginning | |
| | | | | | | | separation | |
| | | | α- | | | | | |
| | K85- | Tween- | Linolenic | Total | | | | Particle |
| | EE | 80 | Acid | Vol. | | | | Size |
| No. | (mg) | (mg) | (mg) | (mg) | Ratio | Pre-conc. | Emulsion | (nm) |
| 101 | 403 | 308 | 106 | 817 | 49:37:12 | Clear | Milky, | — |
| | | | | | | | beginning | |
| | | | | | | | separation | |
| 105 | 452 | 253 | 102 | 807 | 56:31:12 | Clear | Milky, with | — |
| | | | | | | | separation | |
| 109 | 507 | 203 | 112 | 822 | 61:24:13 | Clear | Milky, with | — |
| | | | | | | | separation | |
| | K85- | Tween- | | Total | | | | Particle |
| | EE | 20 | KE85-FA | Vol. | | | | Size |
| No. | (mg) | (mg) | (mg) | (mg) | Ratio | Pre-conc. | Emulsion | (nm) |
| 110 | 398.5 | 300.5 | 98.6 | 797.6 | 49:37:12 | Clear | Milky | |
| | | | | | | | (<10 min | |
| | | | | | | | waiting | |
| | | | | | | | time) | |
| 111 | 448 | 245.9 | 110.4 | 804.3 | 55:30:13 | Unclear | — | — |
| 112 | 498.3 | 197.9 | 106.2 | 802.4 | 62:24:13 | Unclear | — | — |
| | K85- | Tween- | | Total | | | | Particle |
| | EE | 40 | KE85-FA | Vol. | | | | Size |
| No. | (mg) | (mg) | (mg) | (mg) | Ratio | Pre-conc. | Emulsion | (nm) |
| 113 | 405.7 | 303.7 | 105.8 | 815.2 | 49:37:12 | Clear | Milky | — |
| | | | | | | | (<10 min | |
| | | | | | | | waiting | |
| | | | | | | | time) | |
| 114 | 452.8 | 261.6 | 101.8 | 816.2 | 55:32:12 | Clear | Milky | — |
| | | | | | | | (<10 min | |
| | | | | | | | waiting | |
| | | | | | | | time) | |
| 115 | 499 | 212.2 | 114.7 | 825.9 | 60:25:13 | Clear | Milky | — |
| | | | | | | | (<10 min | |
| | | | | | | | waiting | |
| | | | | | | | time) | |
| | K85- | Tween- | | Total | | | | Particle |
| | EE | 60 | KE85-FA | Vol. | | | | Size |
| No. | (mg) | (mg) | (mg) | (mg) | Ratio | Pre-conc. | Emulsion | (nm) |
| 116 | 395 | 296.2 | 100 | 791.2 | 49:37:12 | Clear | Milky | — |
| | | | | | | | (<10 min | |
| | | | | | | | waiting | |
| | | | | | | | time) | |
| 117 | 450.3 | 253.1 | 98.2 | 801.6 | 56:31:12 | Clear | Milky | — |
| | | | | | | | (<10 min | |
| | | | | | | | waiting | |
| | | | | | | | time) | |
| 118 | 500.8 | 206 | 105.7 | 812.5 | 61:25:13 | Clear | Milky | — |
| | | | | | | | (<10 min | |
| | | | | | | | waiting | |
| | | | | | | | time) | |
| | K85- | Tween- | | Total | | | | Particle |
| | EE | 80 | KE85-FA | Vol. | | | | Size |
| No. | (mg) | (mg) | (mg) | (mg) | Ratio | Pre-conc. | Emulsion | (nm) |
| 119 | 402 | 308.3 | 100.8 | 811.1 | 49:38:12 | Clear | Milky, | — |
| | | | | | | | sticky | |
| | | | | | | | (<10 min | |
| | | | | | | | waiting | |
| | | | | | | | time) | |
| 120 | 456.6 | 260.3 | 103.5 | 820.4 | 55:31:12 | Clear | Milky, | — |
| | | | | | | | sticky | |
| | | | | | | | (<10 min | |
| | | | | | | | waiting | |
| | | | | | | | time) | |
| 121 | 502.3 | 202.2 | 104 | 808.5 | 62:25:12 | Clear | Milky, | — |
| | | | | | | | sticky | |
| | | | | | | | (<10 min | |
| | | | | | | | waiting | |
| | | | | | | | time) | |

Of the preconcentrates prepared, formulation number 85 facilitated a load of 60% K85EE into the preconcentrate and gave a stable emulsion in gastric media with a particle size determined to be about 275 nm. Attempts to prepare preconcentrates with saturated fatty acids, stearic acid and decanoic acid failed. Although homogenous preconcentrates could be obtained by heating, a precipitation of stearic acid or decanoic acid was observed upon cooling of the preconcentrate to room temperature.

### Example 2

### Additional Preconcentrates

Additional preconcentrates were prepared to determine an optimized amount of surfactant with K85EE and K85FA. The preconcentrates described in Table 10 were prepared as provided in Example 1. The preconcentrates were visually inspected after mixing and again after being stored for 24 hours at room temperature. Under the Preconcentrate heading, a “clear” designation represents a transparent homogenous mixture; a “turbid” designation represents a nonhomogenous mixture, where some turbidity can be observed by visual inspection. The degree of turbidity was not determined.

TABLE 10
Additional Preconcentrates.

| K85-EE | Tween20 | K85FA | |
|---|---|---|---|
| (mg) | (mg) | (mg) | Preconcentrate |
| 107 | 307 | 62 | Turbid |
| 107 | 307 | 76 | Turbid |
| 107 | 307 | 102 | Turbid |
| 107 | 307 | 200 | Clear |
| 107 | 307 | 401 | Clear |
| 107 | 307 | 803 | Clear |
| 107 | 307 | 1608 | Clear |
| 26 | 300 | 99 | Clear |
| 104 | 300 | 99 | Clear |
| 201 | 300 | 99 | Clear |
| 316 | 300 | 99 | Clear |
| 400 | 300 | 99 | Clear |
| 497 | 300 | 99 | Turbid |
| 618 | 300 | 99 | Turbid |
| 405 | 42 | 101 | Clear |
| 405 | 99 | 101 | Clear |
| 405 | 202 | 101 | Clear |
| 405 | 299 | 101 | Clear |
| 405 | 400 | 101 | Clear |
| 405 | 618 | 101 | Clear |
| 405 | 1000 | 101 | Clear |
| K85-EE | Tween80 | K85FA | |
| (mg) | (mg) | (mg) | Preconcentrate |
| 407 | 306 | 57 | Clear |
| 407 | 306 | 80 | Clear |
| 407 | 306 | 103 | Clear |
| 407 | 306 | 202 | Clear |
| 407 | 306 | 401 | Clear |
| 28 | 299 | 101 | Clear |
| 57 | 299 | 101 | Clear |
| 99 | 299 | 101 | Clear |
| 233 | 299 | 101 | Clear |
| 316 | 299 | 101 | Clear |
| 414 | 299 | 101 | Clear |
| 510 | 299 | 101 | Clear |
| 569 | 299 | 101 | Clear |
| 627 | 299 | 101 | Clear |
| 688 | 299 | 101 | Clear |
| 769 | 299 | 101 | Clear |
| 402 | 32 | 106 | Clear |
| 402 | 126 | 106 | Clear |
| 402 | 229 | 106 | Clear |
| 402 | 326 | 106 | Clear |
| 402 | 410 | 106 | Clear |
| 402 | 997 | 106 | Clear |
| K85-EE | Tween40 | K85FA | |
| (mg) | (mg) | (mg) | Preconcentrate |
| 111 | 311 | 59 | Turbid |
| 111 | 311 | 70 | Clear |
| 111 | 311 | 95 | Clear |
| 111 | 311 | 135 | Clear |
| 111 | 311 | 244 | Clear |
| 111 | 311 | 798 | Clear |
| 111 | 311 | 1567 | Clear |
| 30 | 309 | 98 | Clear |
| 110 | 309 | 98 | Clear |
| 208 | 309 | 98 | Clear |
| 322 | 309 | 98 | Clear |
| 404 | 309 | 98 | Clear |
| 501 | 309 | 98 | Turbid |
| 618 | 309 | 98 | Turbid |
| 408 | 38 | 99 | Clear |
| 408 | 105 | 99 | Clear |
| 408 | 210 | 99 | Clear |
| 408 | 301 | 99 | Clear |
| 408 | 398 | 99 | Clear |
| 408 | 616 | 99 | Clear |
| 408 | 1001 | 99 | Clear |

### Example 3

### Compatibility of Preconcentrates with Solvents

The compatibility of solvents and a preconcentrate having a fixed amount of K85EE and Tween-80 were evaluated. The preconcentrates described in Table 11 were prepared as provided in Example 1, but with the addition of the solvent identified below. The preconcentrates were visually inspected after mixing and again after being stored for 24 hours at room temperature. Under the Preconcentrate heading, a “clear” designation represents a transparent homogenous mixture; a “turbid” designation represents a non-homogenous mixture, where some turbidity can be observed by visual inspection. The degree of turbidity was not determined.

TABLE 11
Compatibility of Solvent and Preconcentrates.

| K85-EE | Tween-80 | 96% ethanol | 96% ethanol | Preconcen- |
|---|---|---|---|---|
| (mg) | (mg) | (mg) | (%) | trate |
| 400 | 110 | 10.7 | 2.1 | Turbid |
| 400 | 110 | 18.7 | 3.5 | Turbid |
| 400 | 110 | 28.4 | 5.3 | Turbid |
| 400 | 110 | 32.1 | 5.9 | Turbid |
| 400 | 110 | 45.7 | 8.2 | Turbid |
| 400 | 110 | 53.5 | 9.5 | Turbid |
| 400 | 110 | 61.5 | 10.8 | Turbid |
| 400 | 110 | 69.8 | 12.0 | Turbid |
| 400 | 110 | 79.9 | 13.5 | Turbid |
| 400 | 110 | 91.3 | 15.2 | Turbid |
| 400 | 110 | 102.5 | 16.7 | Turbid |
| | | Propylene | Propylene | |
| K85-EE | Tween-80 | glycol | glycol | Preconcen- |
| (mg) | (mg) | (mg) | (%) | trate |
| 400 | 110 | 11.1 | 2.1 | Turbid |
| 400 | 110 | 16.7 | 3.2 | Turbid |
| 400 | 110 | 23.1 | 4.3 | Turbid |
| 400 | 110 | 32.9 | 6.1 | Turbid |
| 400 | 110 | 41.5 | 7.5 | Turbid |
| 400 | 110 | 48.6 | 8.7 | Turbid |
| 400 | 110 | 59.9 | 10.5 | Turbid |
| 400 | 110 | 72.9 | 12.5 | Turbid |
| 400 | 110 | 81.5 | 13.8 | Turbid |
| 400 | 110 | 93.5 | 15.5 | Turbid |
| 400 | 110 | 104.6 | 17.0 | Turbid |
| K85-EE | Tween-80 | PEG 300 | PEG 300 | Preconcen- |
| (mg) | (mg) | (mg) | (%) | trate |
| 400 | 110 | 13.9 | 2.7 | Turbid |
| 400 | 110 | 23.7 | 4.4 | Turbid |
| 400 | 110 | 35.6 | 6.5 | Turbid |
| 400 | 110 | 47.1 | 8.5 | Turbid |
| 400 | 110 | 55.0 | 9.7 | Turbid |
| 400 | 110 | 68.7 | 11.9 | Turbid |
| 400 | 110 | 81.8 | 13.8 | Turbid |
| 400 | 110 | 90.3 | 15.0 | Turbid |
| 400 | 110 | 104.0 | 16.9 | Turbid |
| | | Benzyl | Benzyl | |
| K85-EE | Tween-80 | alcohol | alcohol | Preconcen- |
| (mg) | (mg) | (mg) | (%) | trate |
| 400 | 110 | 0 | 0 | Clear |
| 400 | 110 | 11.4 | 2.2 | Turbid |
| 400 | 110 | 18.1 | 3.4 | Turbid |
| 400 | 110 | 30.9 | 5.7 | Clear |
| 400 | 110 | 45.5 | 8.2 | Clear |
| 400 | 110 | 55.6 | 9.8 | Clear |
| 400 | 110 | 66.7 | 11.6 | Clear |
| 400 | 110 | 77.4 | 13.2 | Clear |
| 400 | 110 | 92.1 | 15.3 | Clear |
| 400 | 110 | 99.0 | 16.3 | Clear |
| K85-EE | Tween-80 | Triacetin | Triacetin | Preconcen- |
| (mg) | (mg) | (mg) | (%) | trate |
| 400 | 110 | 12.3 | 2.4 | Turbid |
| 400 | 110 | 24.3 | 4.5 | Turbid |
| 400 | 110 | 35.8 | 6.6 | Turbid |
| 400 | 110 | 45.3 | 8.2 | Turbid |
| 400 | 110 | 57.0 | 10.1 | Turbid |
| 400 | 110 | 68.1 | 11.8 | Turbid |
| 400 | 110 | 80.9 | 13.7 | Turbid |
| 400 | 110 | 90.0 | 15.0 | Turbid |
| 400 | 110 | 101.7 | 16.6 | Turbid |
| | | 1-octa- | 1-octa- | |
| K85-EE | Tween-80 | decanol 99% | decanol 99% | Preconcen- |
| (mg) | (mg) | (mg) | (%) | trate |
| 400 | 110 | 8.6 | 1.7 | Precipitate |
| | | oleyl | oleyl | |
| K85-EE | Tween-80 | alcohol 85% | alcohol 85% | Preconcen- |
| (mg) | (mg) | (mg) | (%) | trate |
| 400 | 100 | 13.0 | 2.5 | Turbid |
| 400 | 100 | 26.5 | 4.9 | Turbid |
| 400 | 100 | 37.3 | 6.8 | Turbid |
| 400 | 100 | 49.5 | 8.8 | Turbid |
| 400 | 100 | 62.6 | 10.9 | Turbid |
| 400 | 100 | 77.7 | 13.2 | Turbid |
| 400 | 100 | 92.2 | 15.3 | Turbid |
| 400 | 100 | 105.7 | 17.2 | Turbid |
| | | 1-tetra- | 1 tetra- | |
| K85-EE | Tween-80 | decanol 97% | decanol 97% | Preconcen- |
| (mg) | (mg) | (mg) | (%) | trate |
| 400 | 100 | 1.7 | 0.3 | Turbid |
| 400 | 100 | 10.3 | 2.0 | Turbid |
| 400 | 100 | 22.7 | 4.3 | Turbid |
| 400 | 100 | 35.8 | 6.6 | Precipitate |
| K85-EE | Tween-80 | glycerol | glycerol | Preconcen- |
| (mg) | (mg) | (mg) | (%) | trate |
| 400 | 100 | 17.7 | 3.4 | Turbid |
| 400 | 100 | 28.0 | 5.2 | Turbid |
| 400 | 100 | 41.7 | 7.6 | Turbid |
| 400 | 100 | 52.8 | 9.4 | Turbid |
| 400 | 100 | 71.2 | 12.3 | Turbid |
| 400 | 100 | 85.4 | 14.3 | Turbid |
| 400 | 100 | 92.3 | 15.3 | Turbid |
| 400 | 100 | 105.7 | 17.2 | Turbid |
| | | Oleic | Oleic | |
| K85-EE | Tween-80 | acid 90% | acid 90% | Preconcen- |
| (mg) | (mg) | (mg) | (%) | trate |
| 400 | 100 | 13.2 | 2.5 | Turbid |
| 400 | 100 | 23.9 | 4.5 | Turbid |
| 400 | 100 | 31.5 | 5.8 | Turbid |
| 400 | 100 | 41.4 | 7.5 | Turbid |
| 400 | 100 | 51.8 | 9.2 | Turbid |
| 400 | 100 | 65.2 | 11.3 | Clear |
| 400 | 100 | 79.8 | 13.5 | Clear |
| 400 | 100 | 87.2 | 14.6 | Clear |
| 400 | 100 | 102.2 | 16.7 | Clear |
| | | 1-docosanol | 1-docosanol | |
| K85-EE | Tween-80 | 98% | 98% | Preconcen- |
| (mg) | (mg) | (mg) | (%) | trate |
| 400 | 100 | 9.6 | 1.8 | Precipitate |

### Example 4

### Characterization of Preconcentrates and SNEDDS/SMEDDS/SEDDS

Preconcentrates A-L described in Table 12 were prepared as provided in Example 1.

TABLE 12
Preconcentrates A-L.

| Precon- | K85-EE | Surfactant | | Total vol. | |
|---|---|---|---|---|---|
| centrate | (mg) | (mg) | FFA (mg) | (mg) | Ratio |
| A | 5002.7 | Tween-20 | Oleic Acid | 10016.4 | 49:36:13 |
| | | 3705.8 | 1307.9 | | |
| B | 5004.9 | Tween-80 | Oleic Acid | 10015.1 | 49:37:13 |
| | | 3707.9 | 1302.3 | | |
| C | 5003.2 | Tween-20 | Ricioleic acid | 10013.4 | 49:36:13 |
| | | 3702.1 | 1308.1 | | |
| D | 5003.5 | Tween-80 | Ricioleic acid | 10010 | 49:36:13 |
| | | 3703.1 | 1303.4 | | |
| E | 5000.4 | Tween-20 | Linoleic acid | 10013.1 | 49:37:13 |
| | | 3707.4 | 1305.3 | | |
| F | 5001 | Tween-80 | Linoleic acid | 10011.3 | 49:37:13 |
| | | 3706 | 1304.3 | | |
| G | 5006.4 | Tween-20 | Erucic acid | 10008.7 | 50:36:12 |
| | | 3702.1 | 1300.2 | | |
| H | 5004.3 | Tween-80 | Erucic acid | 10011.6 | 49:36:13 |
| | | 3704.1 | 1303.2 | | |
| I | 5002.9 | Tween-20 | α-Linolenic acid | 10013.1 | 49:36:13 |
| | | 3700.8 | 1309.4 | | |
| J | 5003.6 | Tween-80 | α-Linolenic | 10017.3 | 49:36:13 |
| | | 3701.6 | acid | | |
| | | | 1312.1 | | |
| K | 5002.9 | Tween-20 | “Pure” EPA-FA + | 10013.1 | 49:36:13 |
| | | 3700.8 | DHA-FA in | | |
| | | | a ratio close | | |
| | | | to K85-EE | | |
| | | | 1309.4 | | |
| L | 5002.9 | Tween-80 | “Pure” EPA-FA + | 10013.1 | 49:36:13 |
| | | 3700.8 | DHA-FA in | | |
| | | | a ratio close | | |
| | | | to K85-EE | | |
| | | | 1309.4 | | |

From Table 12 above, all preconcentrates appeared clear and homogenous, except for the formulation with erucic acid. As such, the preconcentrates can be mixed in any proportion and these mixtures will still form homogenous and clear preconcentrates.

Preconcentrates A-L were also screened for compatibility with various solvents. The outcome of this screening is show in Table 13 below. To 500 mg of preconcentrate, approximately 50 mg of each solvent was added. Preconcentrate A was used for all the solvents. Ethanol was tested in all the preconcentrates. The preconcentrates were visually inspected after mixing and again after being stored for 24 hours at room temperature. Under the Preconcentrate heading, a “clear” designation represents a transparent homogenous mixture; an “unclear” designation represents a nonhomogenous mixture, where some turbidity can be observed by visual inspection. The degree of turbidity was not determined.

TABLE 13
Preconcentrate Compatibility.

| | Preconcentrate | Preconcentrate |
|---|---|---|
| Solvent | A | B-L |
| 96% Ethanol | Clear | Clear |
| Benzyl alcohol | Clear | Nd |
| Propylene glycol | Unclear | Nd |
| Triacetin | Clear | Nd |
| PEG 300 | Unclear | Nd |
| Glycerol | Unclear | Nd |
| 1-octadecanol | Clear, but solid | Nd |
| 99% | | |
| 1-docosanol 98% | Unclear | Nd |
| Oleyl alcohol 85% | Clear | Nd |
| 1-tetradecanol | Clear | Nd |
| 97% | | |

Nd—Not determined.

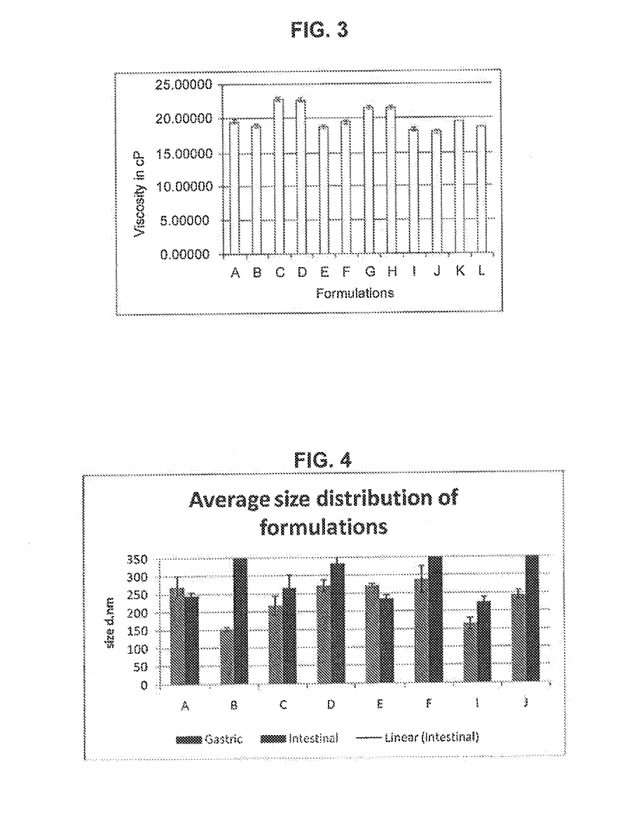

Viscosity can be used as a physical characterization parameter. Viscosity measurements were taken for preconcentrates A-L in triplicate. Generally, the viscosity showed greater sensitivity for the type of fatty acid than for the type of surfactant. FIG. 3 graphically illustrates the viscosity of preconcentrates A-L. Although the viscosity measurements cannot distinguish between Tween 20 versus Tween 80, the viscosity can be impacted by the free fatty acid.

Preconcentrates A-F, I and J were diluted in gastric and intestinal media to form an emulsion (i.e., SNEDDS/SMEDDS/SEDDS). The composition of the gastric media is shown in Table 14, and the composition of the intestinal media is shown in Table 15.

TABLE 14
Gastric Media
Gastric Media

| | |
|---|---|
| Bile salts, Porcine (mM) | 0.08 |
| Lechitin (mM) | 0.02 |
| Sodium chloride (mM) | 34.2 |
| Pepsin (mg/ml) | 0.1 |
| pH | 1.6 (adjust with 1M HCl) |
| Osmolarity (mOsm/kg) | 120 |

TABLE 15
Intestinal Media
Intestinal Media

| | |
|---|---|
| Bile salts, Porcine Bile extract, | 5 |
| Sigma 037K0196 (mM) | |
| Phospholipids, LIPOID S PC from | 1.25 |
| LIPOID AG (mM) | |
| Trizma maleate, Sigma Aldrich, T | 2 |
| 3128 (mM) | |
| Na⁺ (mM) | 150 |

Particle size was measured using a Malvern Zetasizer (Malvern Instrument, Worcestershire, UK) with particle size measuring range of 0.5-6000 nm and Zeta potential of particle range of 3 nm-10 μm. The particle size was measured in triplicate.

For the gastric media, the emulsions were prepared by adding 1 ml of gastric media to 50 mg of the preconcentrate. Table 16 below provides the particle size measurements for preconcentrates A-F, I and J in the gastric media. The particle size measurements in gastric media are also graphically illustrated in FIG. 4.

TABLE 16
Particle size measurements for preconcentrates A-F, I and J in gastric
media.

| | Preconcentrates | | | | | | | |
|---|---|---|---|---|---|---|---|---|
| | A | B | C | D | E | F | I | J |
| Size (nm) | 269.6 | 152.1 | 216.8 | 271 | 271.1 | 287.1 | 165 | 244.3 |
| Standard | 29.63 | 5.141 | 26.24 | 15.94 | 6.208 | 36.71 | 15.87 | 13.67 |
| Deviation | | | | | | | | |

For the intestinal media, the emulsions were prepared by adding the gastric media (100 μl) obtained above to intestinal media (900 μl). Table 17 below provides the particle size measurements for preconcentrates A-F, I and J in the intestinal media. The particle size measurements in intestinal media are also graphically illustrated in FIG. 4.

TABLE 17
Particle size measurements for preconcentrates A-F, I and J in
intestinal media.

| | Preconcentrates | | | | | | | |
|---|---|---|---|---|---|---|---|---|
| | A | B | C | D | E | F | I | J |
| Size (nm) | 245.9 | 2314 | 266.7 | 332.5 | 233.9 | 1891 | 224.3 | 1788 |
| Standard | 7.465 | 2438 | 35.38 | 26.63 | 10.48 | 1936 | 13.56 | 930.5 |
| Deviation | | | | | | | | |

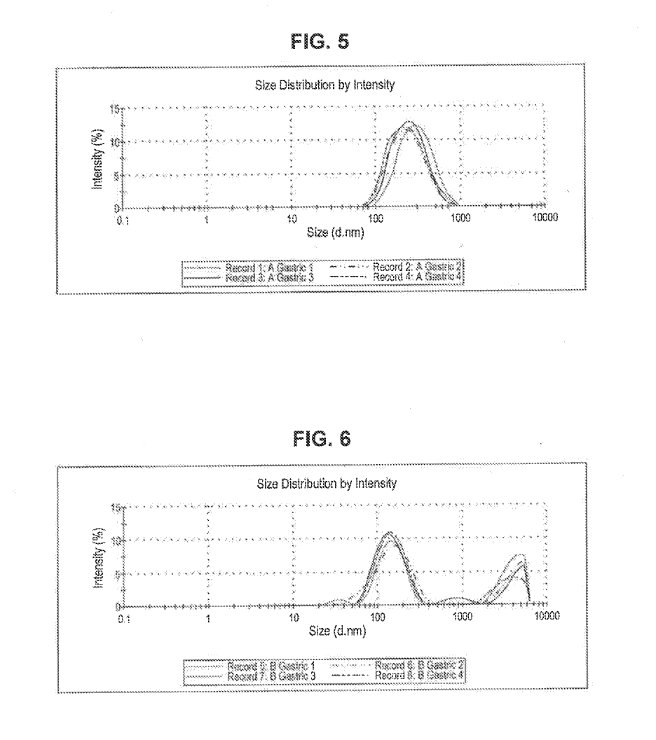

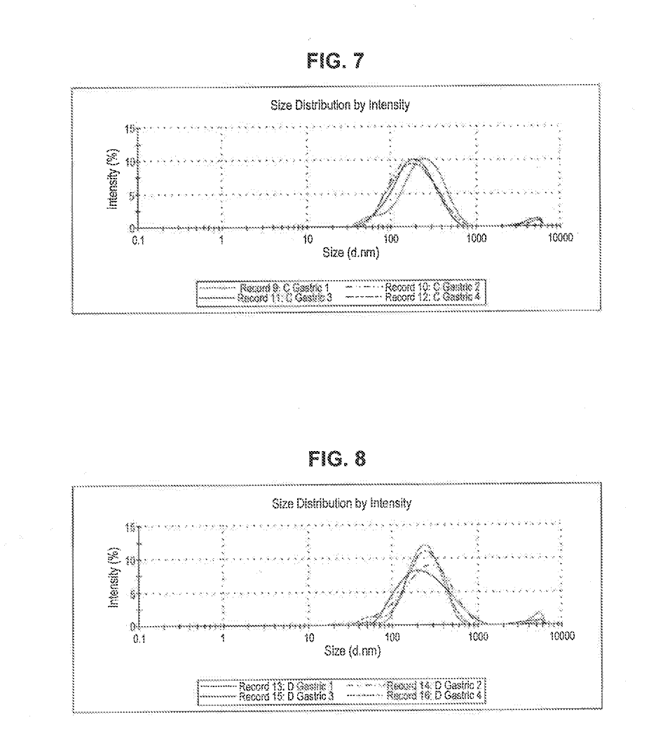

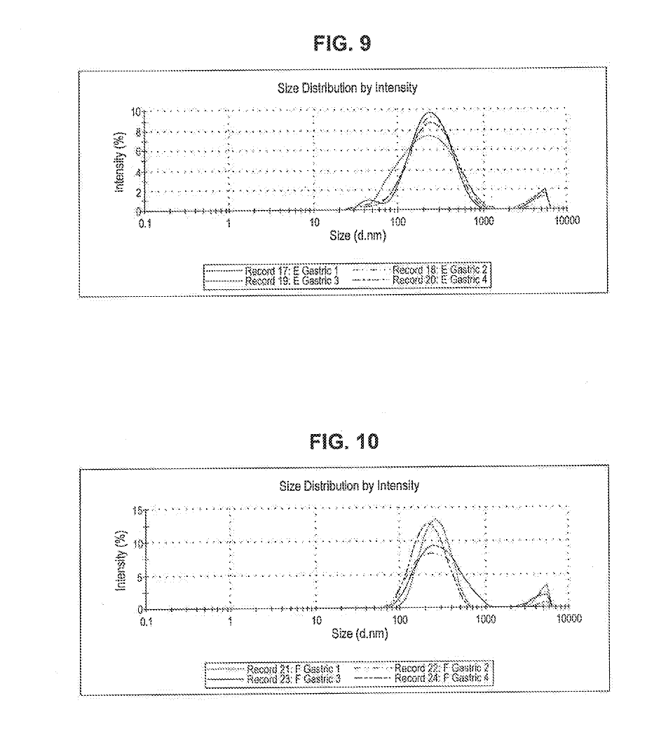

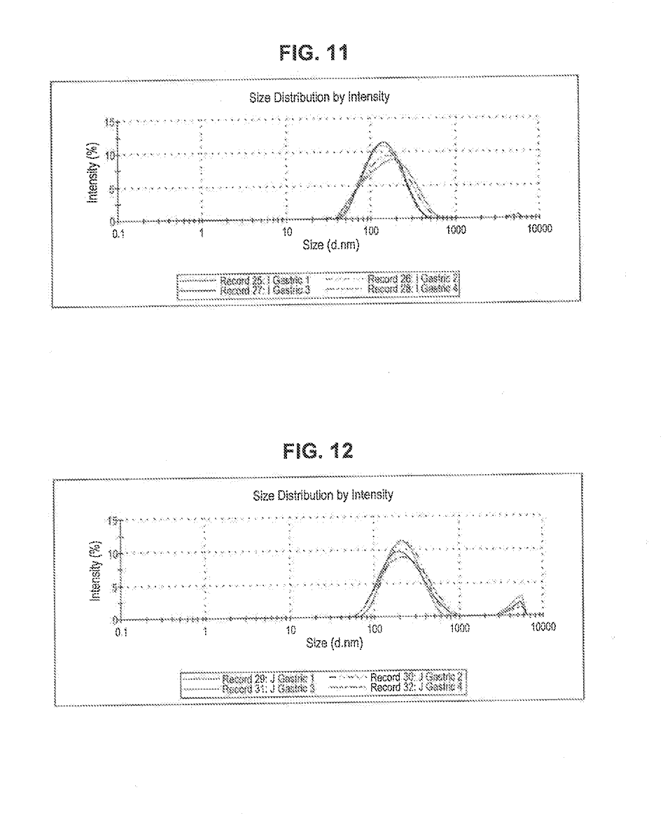

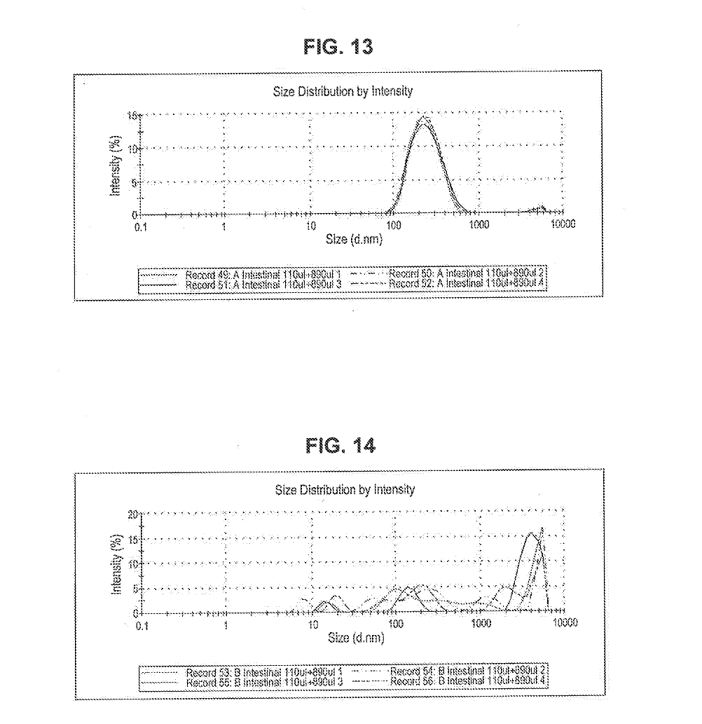

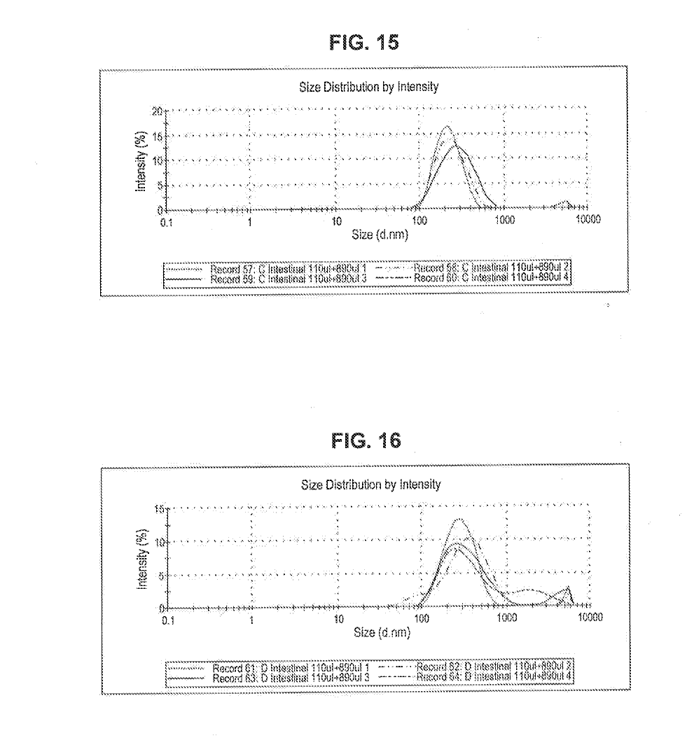

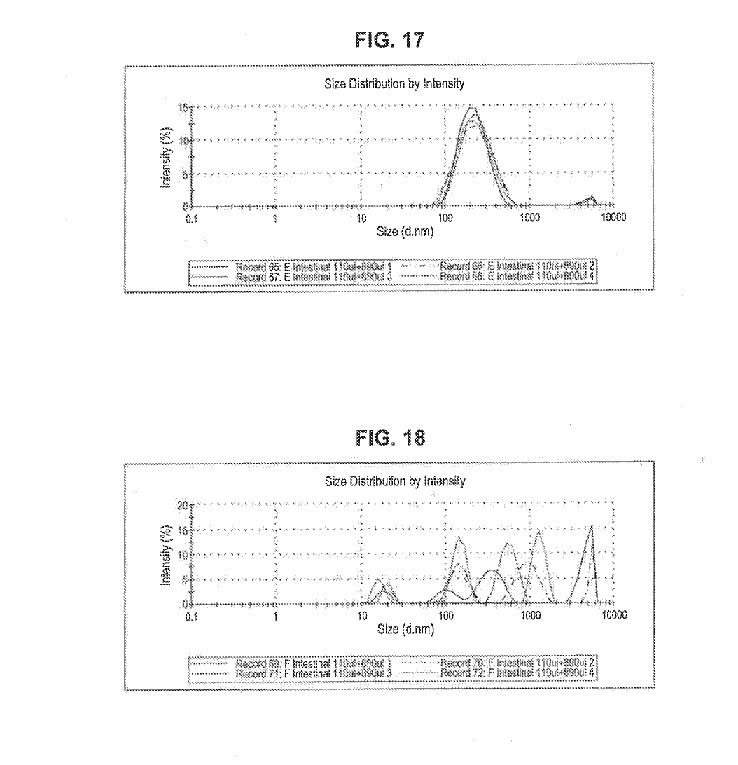

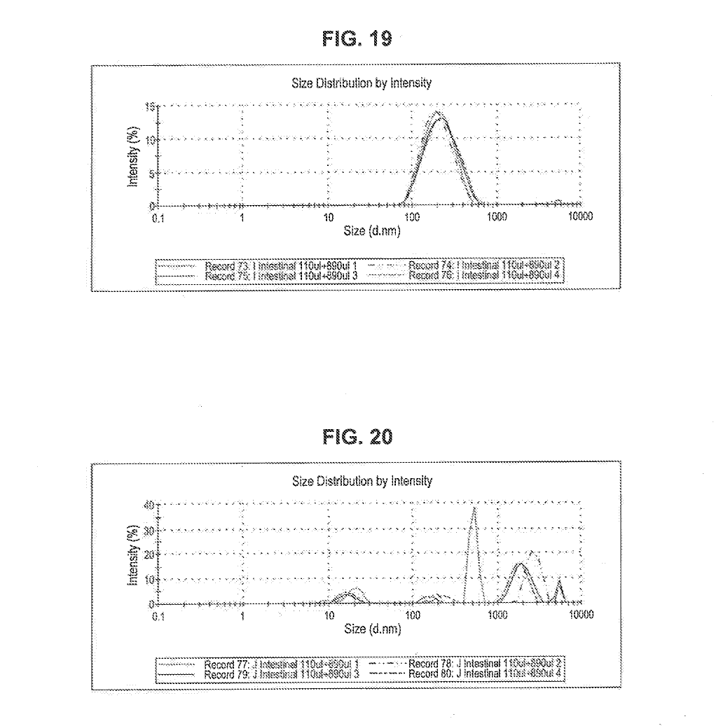

As shown in FIG. 4, intestinal media has a larger impact on the particle size distribution and particularly, preconcentrates comprising Tween 80. That observation has been visualized in FIGS. 5-20. FIGS. 5-20 show the read out from the Malvern zetasizer for four consecutive measurements on the same sample of each respective preconcentrate. All the preconcentrates give near to unimodal particle size distributions in gastric media, whereas only preconcentrates comprising Tween 20 stays unimodal when transferred to intestinal media,

### Example 5

### Lipolysis and Solubilization

Studies were done to analyze the rate of lipolysis (i.e., hydrolysis) and solubilization for different preconcentrates comprising K85EE and different free fatty acids and surfactants. Specifically, four experiments were designed to determine how the amount of surfactant influences the rate and extent of lipolysis and solubilization. The lipolysis was conducted on SMEDDS formulations comprising K85EE.

Materials
Bile salts: Porcine Bile extract (Sigma); contains glycine and taurine conjugates of hyodeoxycholic acid and other bile salts.
Pancreatic lipase, Porcine pancreas (Sigma): contains many enzymes, including amylase, trypsin, lipase, ribonuclease and protease,
Lechitin: Phospholipids (LIPOID S PC from LIPOID AG)
Trizma maleate (Sigma Aldrich)
Tween 20, Molecular Biology Grade (AppliChem Darmstadt), Tween 80 (Fluke)
α-Linoleic acid (Sigma 60%), Oleic acid (Aldrich 90%)
K85-EE and K85-FA

Preconcentrates A-E were prepared as summarized in Table 18.

TABLE 18
Preconcentrates A-E.

| Preconcen- | Fatty acid | | |
|---|---|---|---|
| trate | oil mixture | Free fatty acid | Surfactant |
| A | K85EE (400 mg) | oleic acid | Tween 20 (300 mg) |
| | | (100 mg) | |
| B | K85EE (400 mg) | oleic acid | Tween 20 (75 mg) |
| | | (100 mg) | |
| C | K85EE (500 mg) | linoleic acid | Tween 80 (200 mg) |
| | | (100 mg) | |
| D | K85EE (400 mg) | K85FA (100 mg) | Tween 20 (300 mg) |
| E | K85EE (400 mg) | — | Tween 80 (100 mg) |

Lipolysis General Procedure

The in vitro dynamic lipolysis model developed by Zangenberg et al. (Zangenberg. N. H. et al., Eur. J. Pharm. Sci. 14, 237-244, 2001; Zangenberg, et al., Eur. J. Pharm. Sci. 14, 115-122, 2001) was used with slight modifications. The lipolysis was conducted in a thermostated 600 ml jacketed glass vessel in the presence of porcine bile extract, with continuous addition calcium chloride. The lipase source was porcine pancreatin and the hydrolysis was followed by titration with sodium hydroxide solution (1.0 N) using a pH stat (pH 6.5). The initial composition of the lipolysis media is shown in Table 19.

TABLE 19
Initial composition of lipolysis media.

| Substance | Initial Concentration | |
|---|---|---|
| Pancreatic lipase, Porcine pancreas | 800 | USP units/ml |
| Bile salts, Porcine Bile extract | 5 | mM |
| Phospholipids, LIPOID S PC from LIPOID AG | 1.25 | mM |
| Trizma maleate | 2 | mM |
| Na⁺ | 150 | mM |
| K85-EE | 5.58 | mg/ml |

The final volume in all experiments was 300 ml and the calcium dispensing rate during the experiments was 0,045 mmol/min (0.09 ml/min). In all experiments, the amount of K85-EE added corresponds to 5.58 mg/ml.

To determine the course of K85-EE lipolysis by HPLC, crude samples were withdrawn and acidified with dilute hydrochloric acid. The concentrations of EPA-EE, DHA-EE, EPA-FA and DHA-FA were determined by HPLC in triplicate. Experiments were performed with LC Agilent Technologies 1200 series at a column temperature of 30° C., mobile phase (A) water (0.1% acetic acid) and (13) MeCN (0.1% acetic acid), with gradient: 0 to 8 minutes, from 70% B to 100% B; 8 to 15 minutes, 100% B; 16 to 16 minutes: from 100% B to 70% B, 16 to 20 minutes: 70% B. The flow rate was 0.5 ml/min, UV @ 210 nM, injection volume: 5 μl, and run time: 20 minutes.

Concentrations of EPA ethyl ester (EPA-EE), DHA ethyl ester (DHA-EE), EPA free acid (EPA-FA), and DHA free acid (DHA-FA) were monitored over time and the rate of lipolysis calculated as shown in Table 20 for comparison with Omacor®.

TABLE 20
Lipolysis of EPA and DHA ethyl ester
in comparison to Omacor ®.

| | | | % lipolysis |
|---|---|---|---|
| | EPA-EE lipolysis | DHA-EE lipolysis | K85EE at |
| | (μg/ml/min) | (μg/ml/min) | t = 233 min |
| Omacor ® | 1.5 | 2.3 | 17 |
| A | 2.8 | 4.5 | 41 |
| B | 2.9 | 3.9 | 35 |
| C | 3.7 | 5.0 | 47 |
| D | 3.5 | 5.0 | 55 |
| E | 3.8 | 4.3 | 45 |

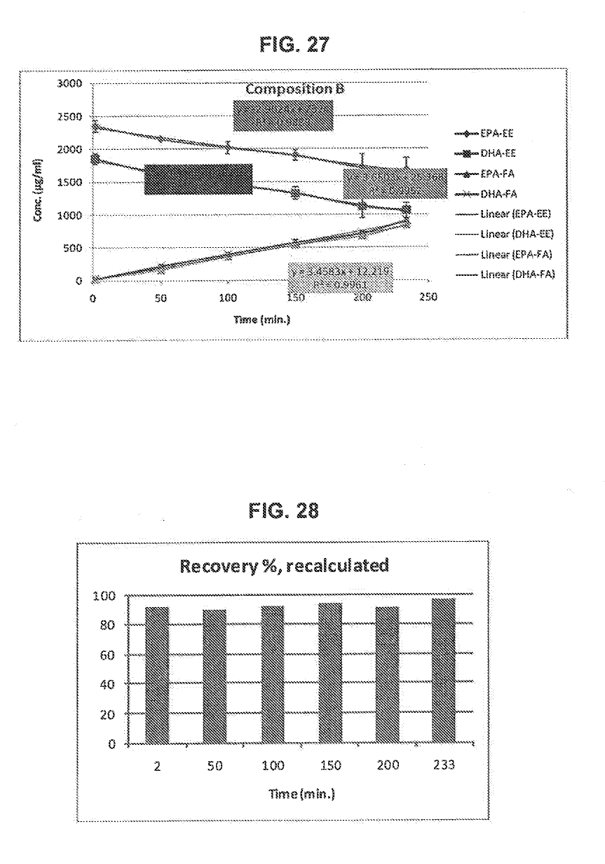

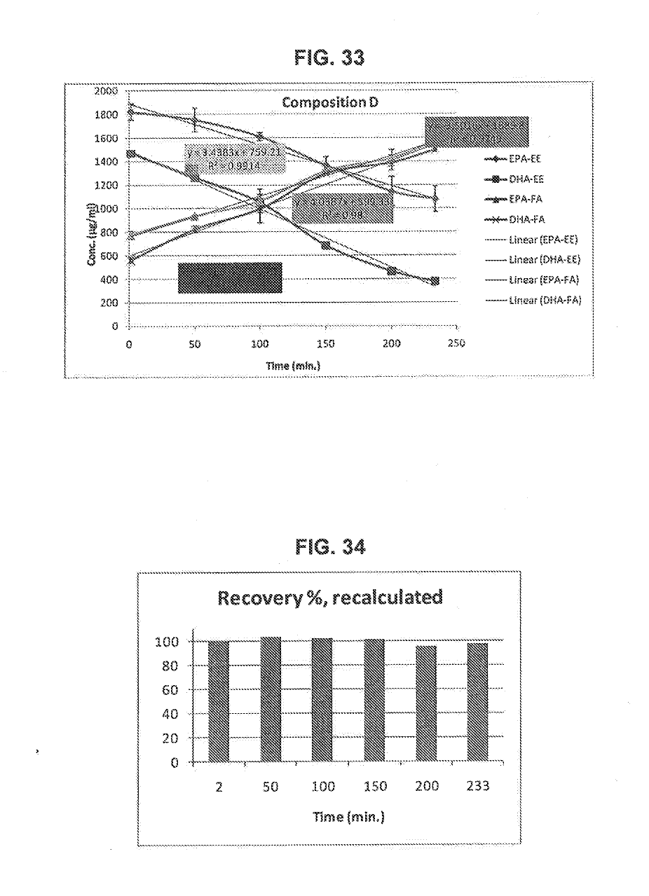

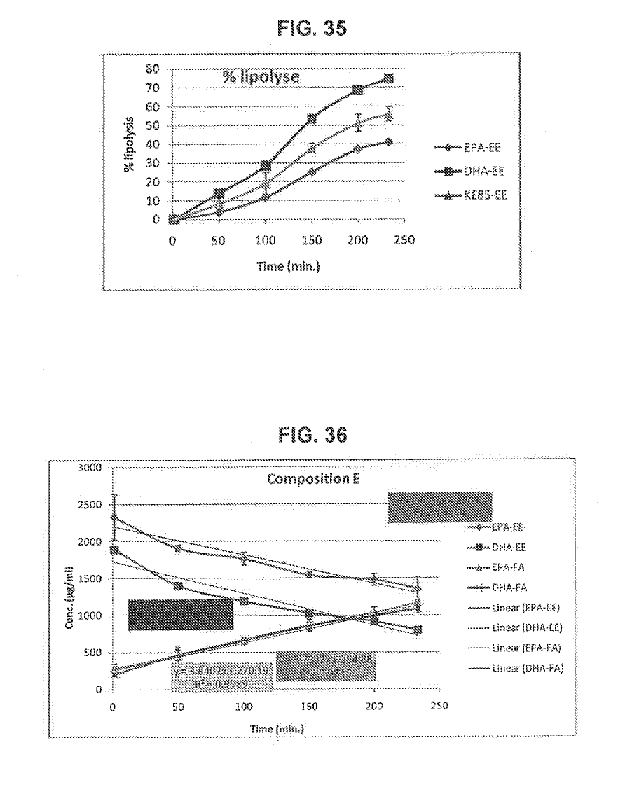

FIGS. 21, 24, 27, 30, 33, and 35 graphically illustrate the disappearance of EPA-EE and DHA-EE and the appearance of EPA-FA and DHA-FA during lipolysis of each respective sample examined. Sample points from 2 minutes to 233 minutes were included in the graphs. In addition, linear regression lines have been included.

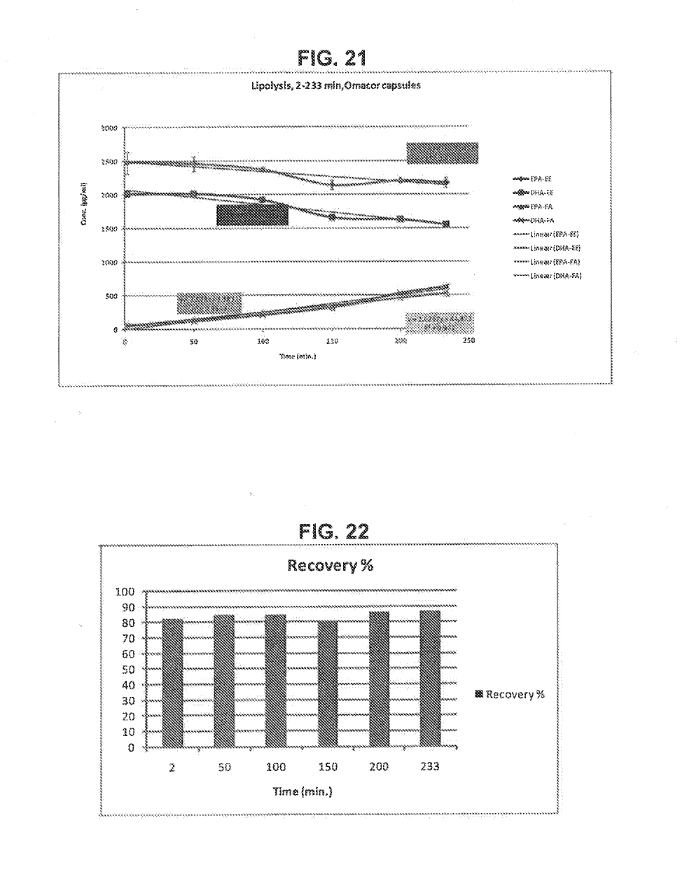

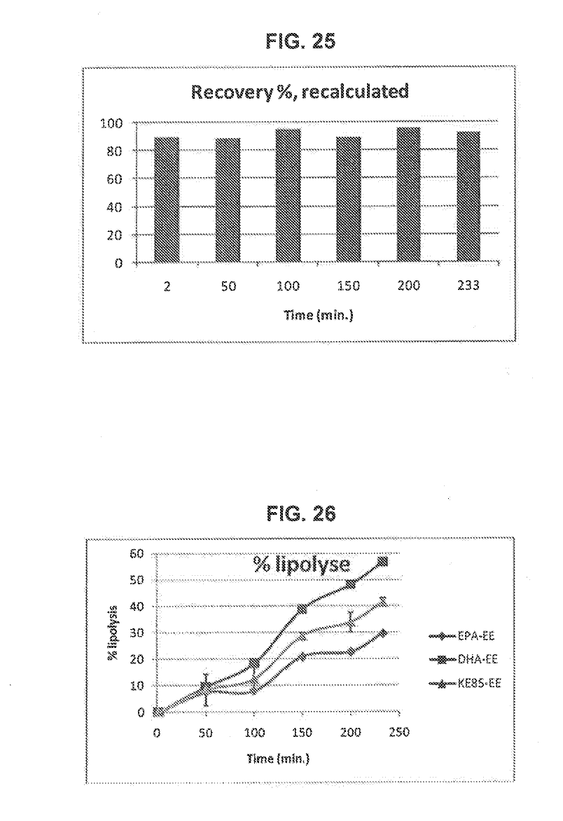

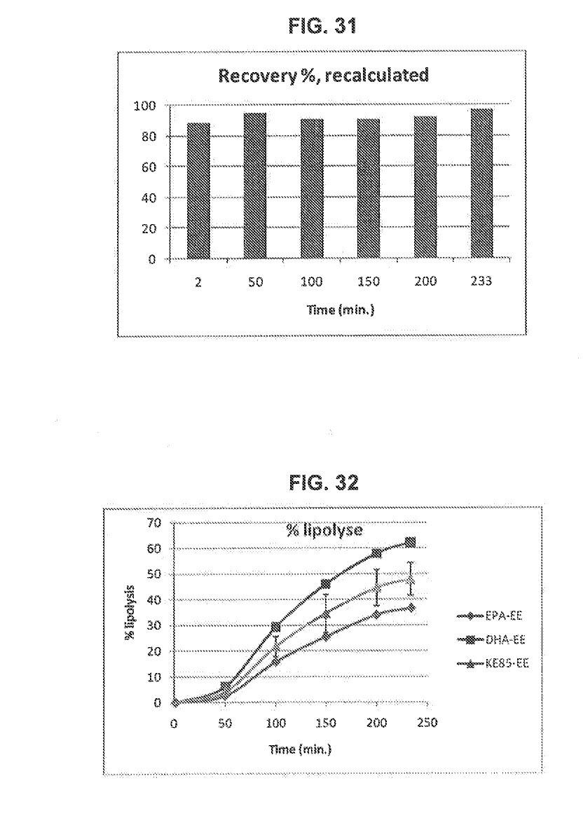

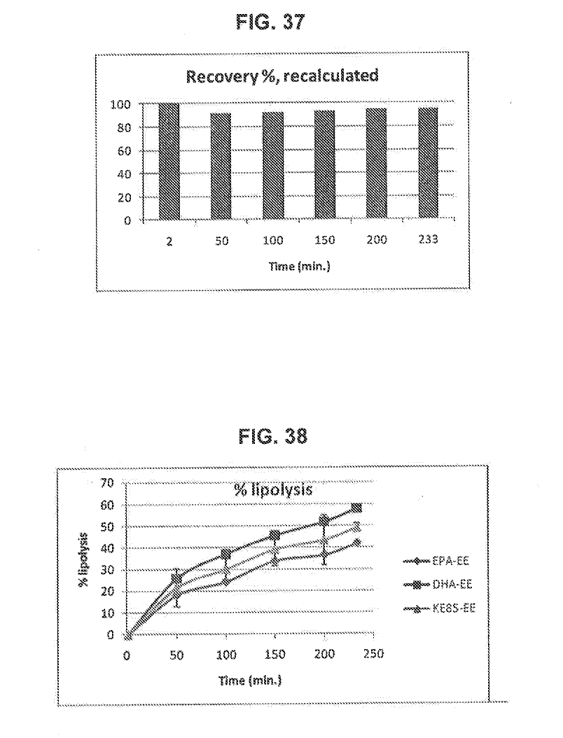

FIGS. 22, 25, 28, 31, 34, and 37 provide the percent recovery of EPA DHA at different time-points for each respective sample examined. Data are given as the sum of EPA-EE, DHA-EE, EPA-FA, and DHA-FA and given as a percentage of theoretical amount 5580 μg/ml.

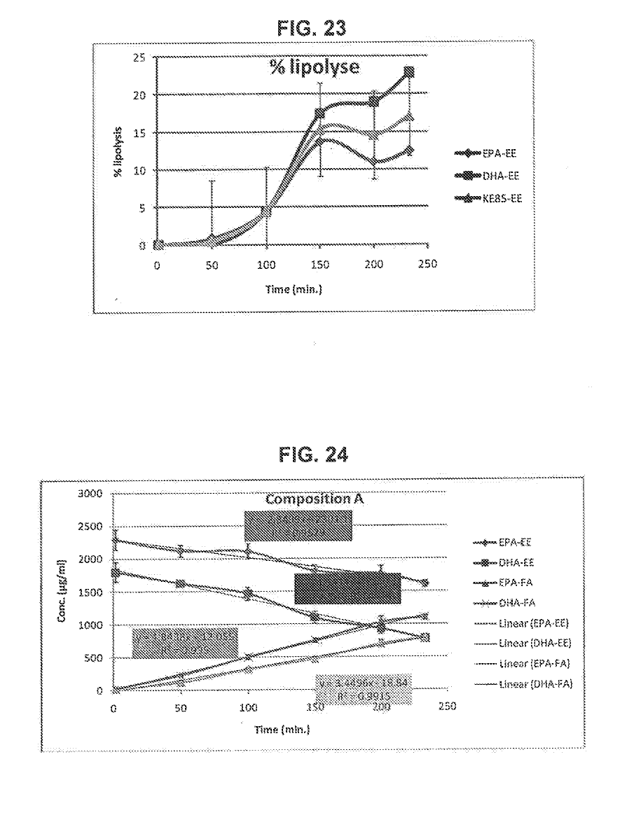

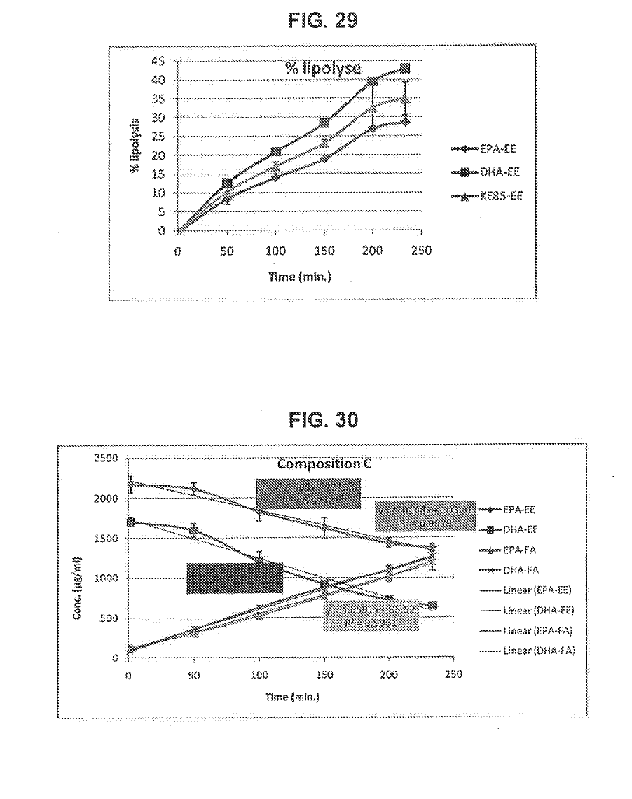

FIGS. 23, 26, 29, 32, 35, and 38 graphically illustrate the percent lipolysis at different time points for EPA-EE, DHA-EE and total K85EE. Values are calculated relative to the total amount of EPA-EE and DHA-EE determined by HPLC after lipolysis for 2 minutes.

### Example 6

### Fat Acid Oil Mixtures of Pharmaceutical Compositions/Preconcentrates

Fatty acid oil mixtures of pharmaceutical compositions or preconcentrates, wherein the fatty acid oil mixture is a K85-EE composition are presented in Table 21.

TABLE 21
Fatty acid oil mixture for pharmaceutical
compositions/preconcentrates

| Fatty acid oil mixture: | Minimum | | Maximum |
|---|---|---|---|
| 1000 mg K85EE fatty acid oil mixture | Value | | Value |
| EPAEE + DHAEE | 800 | mg/g | 880 mg/g |
| EPA EE | 430 | mg/g | 495 mg/g |
| DHA EE | 347 | mg/g | 403 mg/g |
| Total omega-3 EE | >90% | (w/w) | |

EE = ethyl ester

### Example 7

### Table Formulations

Tablets were prepared by immersing the tablet shown in Table 22 in K85EE oil. The mean liquid loading was 160 mg oil/tablet, corresponding to about 72 v/v %. The tablet can also be prepared without a superdisintegrant.

TABLE 22
Tablet compositions

| Tablet composition | Example |
|---|---|
| Neusilin US | 89% |
| Ac-Di-Sol (croscarmellose sodium) = | 10% |
| superdisintegrants | |
| Mg-stearate | 1.0% |

### Example 8

### Novel K85 Tablet Formulation

A tablet formulation is prepared with the components identified in Table 23 by immersing a tablet in a K85EE or AGP oil and an oil in free acid form.

TABLE 23
K83 tablet formulation

| K85 or AGP oil | | |
|---|---|---|
| loading per tablet | Minimum | Maximum value |
| EPA EE and DHA EE | 125 mg | 600 mg |
| Free fatty acid oil | 2% corresponding | 15% corresponding to |
| | to about 2.5 mg | about 90 mg |

### Example 9

### Preparation of SEDDS and SMEDDS

The preconcentrate can be prepared by mixing a fatty acid oil mixture together with at least one surfactant and a free fatty acid.

The preconcentrate can be visually inspected after mixing and again after being stored at 24 hours at room temperature and clear and transparent preconcentrate can be obtained.

To the preconcentrate can then an aqueous medium be added to form an oil-in-water emulsion. The dispersion rate for the formation of the oil-in-water emulsion can be very fast, less than one minute.

The microemulsions formed can then be tested regarding hydrolysis, also called lipolysis.

For example, to determine the course of KE85-EE hydrolysis by HPLC, crude samples can be withdrawn and acidified with dilute hydrochloric acid. The concentrations of EPA-ethyl ester, DHA ethyl ester, EPA-free fatty acid and DHA-free fatty acid can then determined by HPLC.

All samples withdrawn from a non-homogenous phase and some variability in recovery can be expected, especially at early time points.

TABLE 24
Initial concentrations of components in the hydrolysis medium.

| Substance | Initial concentration | |
|---|---|---|
| Pancreatic lipase, Porcine pancreas, Sigma | 800 | USP units/ml |
| 095K1149 | | |
| Bile salts, Porcine Bile extract, Sigma | 5 | mM |
| 037K0196 | | |
| Phospholipids, LIPOID S PC from LIPOID AG | 1.25 | mM |
| Trizma maleate, Sigma Aldrich, T 3128 | 2 | mM |
| Na⁺ | 150 | mM |
| KE85-EE | 5.58 | mg/ml |

An example HPLC analytical method can include the following parameters:

Use of a LC-MS manufactured by AgilentTechnologies and includes a 1200 Series LC and a 6140 Quadropole MS running ChemStation B.04.01 software;

Column: EclipseXDB C18, 2.1×150 mm, 5 μm, Agilent

Column temperature: 25° C.;

Mobile Phase: A: water (0.1% acetic acid), B: MeCN (0.1% acetic acid);

Gradient: 0 to 8 min, from 70% B to 100% B, 8 to 15 minutes: 100% B, from 16 to 16 minutes: from 100% B to 70% B, 16 to 20 minutes: 70% B;

Flow rate: 0.5 ml/min;

UV @ 210 nM;

Injection volume: 25 μl; and

Run time: 20 minutes.

The oil-in-water emulsions can then be further analyzed to determine the particle size of the oil droplets. The particle size can be determined with Malvern Zetasizer (Malvern Instrument, Worcestershire, UK) having particle size measuring range of 0.6-6000 nm and Zeta of particle range of 3 nm-10 μm.

Table 25 shows the components that can be included in pharmaceutical compositions according to the present disclosure

TABLE 25
Sample composition.

| | Pharmaceutical composition |
|---|---|
| Fatty Acid Oil Mixture | K85EE, K85TG or AGP103 drug |
| | substance |
| Surfactant | Tween ®20 or Tween ®40 |
| Free Fatty Acid | (EPA-FA and DHA-FA), |
| | EPA-FA or DHA-FA |
| Total Oil Mixture | 100% by weight |
| 100% by weight | |

Further for example, K85EE omega-3 fatty acid oil and the free fatty acid chosen from K85FA having a EPA:DHA-FA ratio more or less equal to the EPA:DHA-EE ratio in K85EE are exemplified in Table 26.

TABLE 26
Additional compositions.

| Total oil mixture content | | | | Free Fatty | |
|---|---|---|---|---|---|
| [oil:co-surfactant ratio] in | Fatty Acid | | Free Fatty | Acid: EPA and | Total oil |
| SMEDDS/SEDDS | Oil Mixture: | Free Fatty | Acid: EPA-FA | DHA mixture | mixture (by |
| Formulations | K85EE | Acid: K85-FA | or DHA-FA | in FA form | weight) |
| 1.) | 80-95% | | 5-20 w % | | 100 w % |
| 2.) | 70-80% | | 20-30% | | 100 w % |
| 3.) | 50-70% | | 30-50% | | 100 w % |
| 4.) | 50-60% | 40-50% | | | 100 w % |
| 5.) | 60-70% | 30-40% | | | 100 w % |
| 6.) | 70-80% | 20-30% | | | 100 w % |
| 7.) | 80-95% | 5-20% | | | 100 w % |
| 8.) | >80% | | | <20% | 100 w % |
| 9.) | 70-80% | | | 20-30% | 100 w % |
| 10.) | 60-70% | | | 30-40% | 100 w % |
| 11.) | 50-60% | | | 40-50% | 100 w % |
| 12.) | 85-95% | | | 5-15% | 100 w % |
| | | | | EPA > DHA | |
| 13.) | 80-90% | | | 10-20% | 100 w % |
| | | | | EPA > DHA | |
| 14.) | 70-80% | | | 20-30% | 100 w % |
| | | | | EPA > DHA | |
| 15.) | 60-70% | | | 30-40% | 100 w % |
| | | | | EPA > DHA | |

Additionally, the total oil mixtures presented above can be mixed with the surfactant Tween® 20.

Further for example, the K85EE mixed fatty acid composition comprises at least 90% omega-3 ethyl ester fatty acids, and wherein the mixed fatty acid composition comprises from about 80% to about 88% eicosapentaenoic acid ethyl ester and docosahexaenoic acid ethyl ester, by weight of the fatty acid composition.

A collection of ratios between [oil]:[surfactant]:[free fatty acid] (a):b):c)) are illustrated in the table 27. For example, a K85EE or AGP103 oil is used together with a surfactant and a co-surfactant in the [K85EE]:[surfactant]:[free fatty acid] ranges from about 4:2:0.5 to 4:4:2. Thus, the range for the surfactant may be from 2 to 4 and the free fatty acid from 0.5 to 2.

It is also included herein that the K85EE oil mixture presented in Table 27 below can be replaced by a K85TG oil mixture as well as a commercial omega-3 oil concentrate in ethyl ester and/or triglyceride form.

TABLE 27
SMEDDS formulations with Tween20, K85EE, EPA-FA or DHA-FA.

| | | | EPA- | DHA- | | 200 mg |
|---|---|---|---|---|---|---|
| | K85EE | Tween20 | FA | FA | ~K85FA | preconcentrate |
| | (mg) | (mg) | (mg) | (mg) | (mg) | in 10 ml water |
| A | 400 | 400 | 100 | | | emulsion |
| B | 400 | 400 | | 100 | | emulsion |
| C | 400 | 300 | 100 | | | emulsion |
| D | 400 | 300 | | | 100 | emulsion |

### Example 10

### Pharmaceutical Preconcentrate Composition

A pharmaceutical preconcentrate composition was prepared by mixing the following components:

as the fatty acid oil mixture: K85-EE; in an amount of 10.80 g;

as the surfactant: Tween-20 (Molecular Biology Grade, AppliChem Darmstadt, A4974,0250 lot 5N004174) in an amount of 7.44 g;

as the at least one fatty acid: EPA-FA in an amount of 1.53 g; and DHA-FA in an amount of 1.24 g.

With mixing, a transparent homogenous solution was obtained. The density of the formulation was determined to be 1.02 g/ml. The composition was then filled in vials (vial seize=4 ml) each comprising 1.25×1670 mg 2087 mg were prepared, flushed with nitrogen and sealed with parafilm,

### Example 11

### In Vivo Studies in Mini-Pig

Two different formulations were prepared and sent for in-vivo testing. Formulation 1 was prepared according to Example 10 above by mixing the following components: K85EE, Tween20, EPA-FA and DHA-FA in the specified amounts, and Formulation 2 was OMACOR gelatine capsules.

The study was performed in 8 male Göttingen SPF minipigs from Ellegaard Göttingen Minipigs ApS. The animals were housed individually in floor pens (1.2 m²) with sawdust (“Jeluxyl” from Jelu Werk GmbH, Josef Ehrler GmbH & Co KG, Ludwigsmühle, D-73494 Rosenberg, Germany) as bedding.

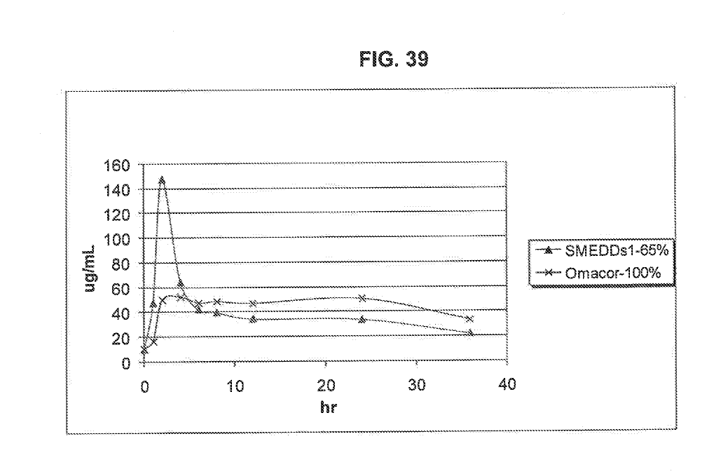

Treatment was performed in a cross-over design. The dose was 2 g per animal. The first day of treatment is designated Day 1. Treatment was performed with a wash out period of at least 10 days between each dosing. Blood samples (n=8) were taken post-dosing. Plasma samples were analysed within 2 weeks for total lipid content of EPA and DHA by a validated LC-MS/MS method. The result presented in FIG. 39 shows the plasma concentration versus time profile of the total lipid concentration of EPA, supporting supra-bioavailability (e.g., great than 40%) for the K85 SMEDDS formulation. A similar result has also been shown for the time profile of total lipid concentration of DHA (not shown in FIG. 39).

### Example 12

### Additional Preconcentrate Compositions

Preconcentrates can be prepared comprising atorvastatin and pharmaceutically acceptable salts, hydrates, solvates, or complexes thereof in omega-3 fatty acid compositions, preconcentrates, and/or SNEDDS/SMEDDS/SEDDS (e.g., self-emulsifying EPA and DHA compositions), wherein atorvastatin is either not soluble in the EPA and DHA oil composition, or soluble but without crystallizing in the mixed oil composition.

TABLE 28
Examples of API combinations according to the present disclosure.

| Mixed fatty acid oil mixture (API I) | Statin (API II) |
|---|---|
| K85EE/Tween 20/K85FA | Atorvastatin (Lipitor ®) 10-80 mg |
| (e.g., about 400/300/110) | |
| K85EE/Tween 20/oleic acid | Atorvastatin (Lipitor ®) 10-80 mg |
| (e.g., about 400/300/100) | |
| K85EE/Tween 20/oleic acid | Atorvastatin (Lipitor ®) 10-80 mg |
| (e.g., about 400/75/100) | |
| K85EE/Tween 20/PRB- | Atorvastatin (Lipitor ®) 10-80 mg |
| 01005/lecithin | |
| (e.g., about 400/250/100/100) | |
| K85FA/Tween 20/lecithin | Atorvastatin (Lipitor ®) 10-80 mg |
| (e.g., about 450/200/50) | |
| K85EE/Tween 80 | Atorvastatin (Lipitor ®) 10-80 mg |
| (e.g., about 400/100) | |
| K85EE/at least one surfactant | Atorvastatin (Lipitor ®) 10-80 mg |
| such as Tween 20 | |
| (e.g., about 400/100) | |

### Example 13

### Formulations Comprising Atorvastatin

The following atorvastatin salts, hydrates, and cyclodextrin (CD) complexes were prepared for testing in compositions, preconcentrates, and/or SNEDDS/SMEDDS/SEDDS according to the present disclosure:

Sample 1: Amorphous atorvastatin calcium

Sample 2: Atorvastatin meglumin salt

Sample 3: Atorvastatin meglumin CD complex

Sample 4: Atorvastatin calcium CD complex

Sample 5: Atorvastatin calcium trihydrate

Sample 6: Atorvastatin calcium

The following atorvastatin formulations were also prepared for testing in compositions, preconcentrates, and/or SNEDDS/SMEDDS/SEDDS according to the present disclosure.

Sample 1: Atorvastatin calcium amorphous, (Drug Discovery Laboratory AS, No)

Sample 2: Atorvastatin meglumin salt (Drug Discovery Laboratories AS, No), batch 010-85.

Sample 3 Atorvastatine meglumin beta-CD complex (Drug Dicovery Laboratories AS, No).

Sample 4: Atorvastatine calcium beta-CD complex (Drug Discovery Laboratories AS, No)

Sample 5: Atorvastatin free acid, batch EXP-10-AB7860-1

Sample 6: Atorvastatine-crysmeb complex crystallized: BF-10-AB7862-CA-1.

Sample 7: Atorvastatine-beta cyclodextrin complex crystallized: BF-10-A87862-BA-1.

Sample 8: Atorvastatine-kleptose complex crystallized: BF-10-AB7862-KA-1:

Sample 9: Atorvastatine-crysmeb complex: BF-10-AB7857-CA-B.

Sample 10: Atorvastatine-beta cyclodextrin complex: BF-10-AB7857-BA-B.

Sample 11: Atorvastatine-kleptose complex: BF-10-AB7882-KA-B

Sample 12: Atorvastatine-crysmeb complex crystallized BF-10-AB7862-CA-

Sample 13: Atorvastatine-beta cyclodextrin complex crystallized 2BF-10-A87862-BA-2

Sample 14: Atorvastatine-kleptose complex crystallised: BF-10-AB7862-KA-2

Sample 14: Atorvastatine-kleptose complex crystallised: BF-10-A87882-KA-2

Cyclodextrin complexes of atorvastatine calcium trihydrate were prepared by evaporating a solution of a mixture of atorvastatine and the appropriate cyclodextrin. The purity of salts, free acids and cyclodextrin complexes to be included in later solubility and stability studies was determined by HPLC,

### Example 14

### Additional Preconcentrate Compositions

The following additional preconcentrate compositions were prepared and evaluated visually, which are summarized in Table 29. To these preconcentrate compositions, a statin may be added, such as, for example, atorvastatin, rosuvastatin, simvastatin, and pharmaceutically acceptable salts, hydrates, solvates, or complexes thereof:

Under the Preconcentrate heading, a “homogeneous” designation represents that a homogenous mixture was formed. The % in the “% K85-FA” heading represents the weight percentage of K85-FA in the preconcentrate composition.

TABLE 29
Examples of additional preconcentrate compositions.

| | K85- | | | | % | |
|---|---|---|---|---|---|---|
| Ref. | FA | | Tween | Lechitin | K85- | |
| No. | (mg) | K85-EE (mg) | 20 (mg) | (mg) | FA | Preconcentrate |
| 1 | 200 | 100 | 200 | | 40 | homogeneous |
| 2 | 200 | 150 | 200 | | 36 | homogeneous |
| 3 | 200 | 200 | 200 | | 33 | homogeneous |
| 4 | 200 | 250 | 200 | | 31 | homogeneous |
| 5 | 200 | 300 | 200 | | 29 | homogeneous |
| 6 | 200 | 100 | 200 | 100 | 33 | homogeneous |
| 7 | 200 | 150 | 200 | 100 | 31 | homogeneous |
| 8 | 200 | 200 | 200 | 100 | 29 | homogeneous |
| 9 | 200 | 250 | 200 | 100 | 27 | homogeneous |
| 10 | 200 | 300 | 200 | 100 | 25 | homogeneous |
| 11 | 200 | 200 | 200 | | 33 | homogeneous |
| 12 | 200 | 200 | 250 | | 31 | homogeneous |
| 13 | 200 | 200 | 300 | | 29 | homogeneous |
| 14 | 200 | 200 | 350 | | 27 | homogeneous |
| 15 | 200 | 200 | 400 | | 25 | homogeneous |

Preconcentrates can be prepared comprising atorvastatin, rosuvastatin, simvastatin, and pharmaceutically acceptable salts, hydrates, solvates, or complexes thereof in preconcentrates and/or SNEDDS/SMEDDS/SEDDS self-emulsifying EPA and DHA compositions), wherein the atorvastatin, rosuvastatin, simvastatin, and pharmaceutically acceptable salts, hydrates, solvates, or complexes thereof are either not soluble in the EPA and DHA oil composition, or soluble but without crystallizing in the mixed oil composition.

### Example 15

### Additional Preconcentrate Compositions

The following additional preconcentrate compositions were prepared and evaluated visually, which are summarized in Tables 30 and 31. To these preconcentrate compositions, a statin may be added, such as, for example, atorvastatin, rosuvastatin, simvastatin, and pharmaceutically acceptable salts, hydrates, solvates, or complexes thereof.

Under the Preconcentrate heading, a “homogeneous” designation represents that a homogenous mixture was formed, and a “turbid” designation represents that a nonhomogeneous mixture was formed, where some turbidity can be observed by visual inspection. The degree of turbidity was not determined.

The “%” in the “% K85-EE” heading represents the weight percentage of K85-EE in the preconcentrate composition.

TABLE 30
Examples of additional preconcentrate compositions comprising Tween 20
and 80, and oleic acid,

| | Tween | Tween | | | | |
|---|---|---|---|---|---|---|
| Ref. | 20 | 80 | Oleic | K85-EE | % K85- | |
| No. | (mg) | (mg) | acid (mg) | (mg) | EE | Preconcentrate |
| 101 | 450 | 450 | 100 | 1200 | 55 | homogeneous |
| 101a | 450 | 450 | 75 | 1000 | 51 | homogeneous |
| 101b | 450 | 450 | 50 | 1000 | 51 | homogeneous |
| 101c | 400 | 500 | 100 | 1100 | 52 | homogeneous |
| 101d | 350 | 550 | 100 | 1100 | 52 | homogeneous |
| 101e | 300 | 600 | 100 | 1100 | 52 | homogeneous |
| 101f | 200 | 700 | 100 | 1100 | 52 | homogeneous |
| 101g | 100 | 800 | 100 | 1100 | 52 | homogeneous |
| 101h | 300 | 300 | 400 | 825 | 45 | homogeneous |
| 101i | 450 | 450 | 100 | 900 | 47 | homogeneous |
| 101j | 500 | 400 | 100 | 900 | 47 | homogeneous |
| 101k | 550 | 350 | 100 | 900 | 47 | homogeneous |
| 101l | 600 | 300 | 100 | 900 | 47 | homogeneous |
| 101m | 700 | 200 | 100 | 900 | 47 | homogeneous |
| 101n | 700 | 200 | 75 | 1000 | 51 | homogeneous |
| 101o | 700 | 200 | 50 | 1000 | 51 | homogeneous |
| 101p | 450 | 450 | 100 | 675 | 40 | homogeneous |
| 101q | 450 | 450 | 100 | 700 | 41 | homogeneous |
| 101r | 450 | 450 | 75 | 700 | 42 | homogeneous |
| 101s | 450 | 450 | 50 | 700 | 42 | homogeneous |
| 101t | 450 | 450 | 100 | 800 | 44 | homogeneous |
| 101u | 450 | 450 | 75 | 800 | 45 | homogeneous |
| 101v | 450 | 450 | 50 | 800 | 46 | homogeneous |

TABLE 31
Examples of additional preconcentrate compositions, comprising Tween
20 and 80, and K85-FA.

| | Tween | | | | | |
|---|---|---|---|---|---|---|
| Ref. | 20 | Tween 80 | K85-FA | K85-EE | % K85- | |
| No. | (mg) | (mg) | (mg) | (mg) | EE | Preconcentrate |
| 102 | 450 | 450 | 100 | 1200 | 55 | homogeneous |
| 102a | 450 | 450 | 75 | 1000 | 51 | homogeneous |
| 102b | 450 | 450 | 50 | 1000 | 51 | homogeneous |
| 102c | 400 | 500 | 100 | 1100 | 52 | homogeneous |
| 102d | 350 | 550 | 100 | 1100 | 52 | homogeneous |
| 102e | 300 | 600 | 100 | 1100 | 52 | homogeneous |
| 102f | 200 | 700 | 100 | 1100 | 52 | homogeneous |
| 102g | 100 | 800 | 100 | 1100 | 52 | homogeneous |
| 102h | 300 | 300 | 400 | 825 | 45 | homogeneous |
| 102i | 450 | 450 | 100 | 900 | 47 | homogeneous |
| 102j | 500 | 400 | 100 | 900 | 47 | homogeneous |
| 102k | 550 | 350 | 100 | 900 | 47 | homogeneous |
| 102l | 600 | 300 | 100 | 900 | 47 | homogeneous |
| 102m | 700 | 200 | 100 | 900 | 47 | homogeneous |
| 102n | 700 | 200 | 75 | 1000 | 51 | homogeneous |
| 102o | 700 | 200 | 50 | 1000 | 51 | homogeneous |
| 102p | 450 | 450 | 100 | 675 | 40 | homogeneous |
| 102q | 450 | 450 | 100 | 700 | 41 | homogeneous |
| 102r | 450 | 450 | 75 | 700 | 42 | homogeneous |
| 102s | 450 | 450 | 50 | 700 | 42 | homogeneous |
| 102t | 450 | 450 | 100 | 800 | 44 | homogeneous |
| 102u | 450 | 450 | 75 | 800 | 45 | homogeneous |
| 102v | 450 | 450 | 50 | 800 | 46 | homogeneous |

Preconcentrates can be prepared comprising atorvastatin, rosuvastatin, simvastatin, and pharmaceutically acceptable salts, hydrates, solvates, or complexes thereof in omega-3 fatty acid compositions, preconcentrates, and/or SNEDDS/SMEDDS/SEDDS (e.g., self-emulsifying EPA and DHA compositions), wherein the atorvastatin, rosuvastatin, simvastatin, and pharmaceutically acceptable salts, hydrates, solvates, or complexes thereof are either not soluble in the EPA and DHA oil composition, or soluble but without crystallizing in the mixed oil composition,

### Example 16

### Statin Solubility in a Preconcentrate

The solubility of 3 different stains in a formulation was evaluated. The following materials and equipment were used:
HPLC: Dionex Ultimate 3000
Column: Phenomenex luna 5μ C18(2) 100A 125×4.0 mm (batch no.: 00E-4252-D0)
Atorvastatin calcium trihydrate: AvaChem Scientific (Lot. no: AF803)
Simvastatin: Toronto Research Chemicals (8-ABY-98-1)
Rosuvastatin calcium: Sequoia Research Pruducts (control no: 04010113265r)
Acetonitrile: HiperSolv Isocratic grade
Acetic acid: Glacial 100%
K85-EE: batch no. 2100033
Tween 20: lot. no. 5N004174
Cremophor: lot. no. 35-2026

A preconcentrate composition was prepared according to Table 32 below.

TABLE 32
Preparation of a preconcentrate composition

| Ref. No. | K85-EE (mg) | Tween 20 (mg) | Oleic acid (mg) |
|---|---|---|---|
| 1 | 400 | 300 | 100 |

Procedure for Making the Four Formulations

Approximately 1000 mg of preconcentrate composition 1 in Table 32 was added to three Eppendorf tubes (n=2). To the first tube, 30 mg/gram atorvastatin calcium trihydrate was added. To the second tube, 30 mg/gram rosuvastatin calcium was added. To the third tube, 100 mg/gram simvastatin was added. The tubes were then incubated in an end-over-end rotator for 48 hours.

After 48 hours of incubation, the samples were centrifuged at 15.000 rpm for 10 min. A sample of approximately 200 mg was withdrawn from each tube. Each sample was added to an Eppendorf tube containing 1000 μl 2-propanol/MeCN (25/75). This solution was further diluted 100 μl+900 μl 2-propanol/MeCN (25/75), followed by analysis by HPLC.

The following HPLC parameters were used:
Injection volume: 5 μl
Column temp.: 30° C.
Detection (UV): 254 nm
Flow rate: 0.5 ml
Used column: Phenomenex luna 5μ C18(2) 100A 125×4.0 mm (batch no.: 00E-4252-D0)
Solvents:
A: 1 ml acetic acid added to 1000 ml with milli Q water
B: 1 ml acetic acid added to 1000 ml with MeCN

HPLC Gradient:

| Time (min) | Solvent A (%) | Solvent B (%) |
|---|---|---|
| 0 | 30 | 70 |
| 8 | 0 | 100 |
| 15 | 0 | 100 |
| 16 | 30 | 0 |
| 20 | 30 | 70 |

By looking at the area, one can determine the solubility of the statin in the preconcentrate composition. For example, the atorvastatin calcium trihydrate exhibited the following HPLC results summarized in Table 33. From the data, the solubility of the statins in mg per gram of formulation was calculated. The rosuvastatin calcium and simvastatin exhibited the HPLC results summarized in Tables 34 and 35, respectively,

TABLE 33
Results for Atorvastatin Calcium Trihydrate

| | | | mg | | | Standard |
|---|---|---|---|---|---|---|
| | | | weighed | | Average | Deviation |
| Ref No. | Area | μg/ml | in 1000 μl | mg/g | mg/g | (mg/g) |
| 1 | 131 | 3243.0 | 177 | 15.57 | 15.44 | 0.17 |
| | 126 | 3129.4 | 174 | 15.32 | | |

TABLE 34
Results for Rosuvastatin Calcium

| | | | mg | | | Standard |
|---|---|---|---|---|---|---|
| | | | weighed | | Average | Deviation |
| Ref No. | Area | μg/ml | in 1000 μl | mg/g | mg/g | (mg/g) |
| 1 | 41 | 1394.8 | 169.7 | 7.03 | 7.91 | 1.25 |
| | 50 | 1709.8 | 166.6 | 8.80 | | |

TABLE 35
Results for Simvastatin

| | | | mg | | | Standard |
|---|---|---|---|---|---|---|
| | | | weighed | | Average | Deviation |
| Ref No. | Area | μg/ml | in 1000 μl | mg/g | mg/g | (mg/g) |
| 1 | 406 | 8748.1 | 174.8 | 42.60 | 44.06 | 2.07 |
| | 410 | 8836.1 | 166.4 | 45.53 | | |

1-200. (canceled)

201. A pharmaceutical composition comprising:
a fatty acid oil mixture comprising at least 75% eicosapentaenoic acid (EPA) and docosahexaenoic acid (DHA), by weight of the fatty acid oil mixture, wherein the EPA and DHA are in a form chosen from ethyl ester and triglyceride;
at least one free fatty acid; and
at least one statin or pharmaceutically acceptable salt, hydrate, solvate, or complex thereof.

202. The composition according to claim 201, wherein of the at least 75% EPA and DHA of the fatty acid oil mixture, at least 95% is EPA.

203. The composition according to claim 201, wherein of the at least 75% EPA and DHA of the fatty acid oil mixture, at least 95% is DHA.

204. The composition according to claim 201, wherein the fatty acid oil mixture comprises at least 90% omega-3 fatty acids, by weight of the fatty acid oil mixture.

205. The composition according to claim 201, wherein the fatty acid oil mixture further comprises at least one other fatty acid other than EPA and DHA in a form chosen from ethyl ester and triglyceride, wherein the at least one other fatty acid is chosen from α-linolenic acid (ALA), heneicosapentaenoic acid (HPA), docosapentaenoic acid (DPA), eicosatetraenoic acid (ETA), eicosatrienoic acid (ETE), stearidonic acid (STA), linoleic acid, α-linolenic acid (ALA), gamma-linolenic acid (GLA), arachidonic acid (AA), osbond acid, and mixtures thereof.

206. The composition according to claim 201, wherein the at least one free fatty acid is chosen from EPA, DHA, ALA, HPA, DPA, ETA, ETE, STA, linoleic acid, GLA. AA, osbond acid, oleic acid, ricinoleic acid, erucic acid, and mixtures thereof.

207. The composition according to claim 201, wherein the at least one statin or pharmaceutically acceptable salt, hydrate, solvate, or complex thereof is chosen from atorvastatin, cerivastatin, fluvastatin, itavastatin, lovastatin, mevastatin, rosuvastatin, simvastatin, pravastatin, pitavastatin, and pharmaceutically acceptable salts, hydrates, solvates, and complexes thereof.

208. The composition according to claim 207, wherein the at least one statin or pharmaceutically acceptable salt, hydrate, solvate, or complex thereof is chosen from simvastatin, atorvastatin, rosuvastatin, and pharmaceutically acceptable salts, hydrates, solvates, and complexes thereof.

209. The composition according to claim 208, wherein the at least one statin or pharmaceutically acceptable salt, hydrate, solvate, or complex thereof comprises atorvastatin or a calcium salt of atorvastatin.

210. The composition according to claim 201, wherein the at least one statin or pharmaceutically acceptable salt, hydrate, solvate, or complex thereof is present in an amoung ranging from about 10 mg to about 80 mg.

211. The composition according to claim 201, wherein the fatty acid oil mixture is derived from at least one oil chosen from marine oil, algae oil, plant-based oil, and microbial oil.

212. The composition according to claim 211, wherein the marine oil is a purified fish oil.

213. The composition according to claim 201, wherein the fatty acid oil mixture comprises from about 50% to about 95% by weight and the at least one free fatty acid comprises from about 5% to about 50% by weight, each relative to the total weight of the composition.

214. The composition according to claim 201, wherein the EPA:DHA weight ratio of the fatty acid oil mixture ranges from about 1:10 to 10:1, from about 1:8 to 8:1, from about 1:6 to 6:1, from about 1:5 to 5:1, from about 1:4 to 4:1, from about 1:3 to 3:1, from about 1:2 to 2:1, from about 1:1 to 2:1, or from about 1:2 to 1:3.

215. The composition according to claim 201, further comprising at least one antioxidant.

216. The composition according to claim 201, further comprising at least one superdisintegrant chosen from crosscarmelose, crospovidone, and sodium starch glycolate, and wherein the composition is loaded into a tablet.

217. The composition according to claim 201, wherein the composition is in the form of a gelatin capsule.

218. A pharmaceutical preconcentrate comprising:
a fatty acid oil mixture comprising at least 75% eicosapentaenoic acid (EPA) and docosahexaenoic acid (DHA), by weight of the fatty acid oil mixture, wherein the EPA and DHA are in a form chosen from ethyl ester and triglyceride;
at least one free fatty acid;
at least one surfactant; and
at least one statin or pharmaceutically acceptable salt, hydrate, solvate, or complex thereof.

219. The preconcentrate according to claim 218, wherein of the at least 75% EPA and DHA of the fatty acid oil mixture, at least 95% is EPA.

220. The preconcentrate according to claim 218, wherein of the at least 75% EPA and DHA of the fatty acid oil mixture, at least 95% is DHA.

221. The preconcentrate according to claim 218, wherein the fatty acid oil mixture comprises at least 90% omega-3 fatty acids, by weight of the fatty acid oil mixture.

222. The preconcentrate according to claim 218, wherein the fatty acid oil mixture further comprises at least one other fatty acid other than EPA and DHA in a form chosen from ethyl ester and triglyceride chosen from α-linolenic acid (ALA), heneicosapentaenoic acid (HPA), docosapentaenoic acid (DPA), eicosatetraenoic acid (ETA), eicosatrienoic acid (ETE), stearidonic acid (STA), linoleic acid, α-linolenic acid (ALA), gamma-linolenic acid (GLA), arachidonic acid (AA), osbond acid, and mixtures thereof.

223. The preconcentrate according to claim 218, wherein the at least one free fatty acid is chosen from EPA, DHA, ALA, HPA, DPA, ETA, ETE, STA, linoleic acid, GLA, AA, osbond acid, oleic acid, ricinoleic acid, erucic acid, and mixtures thereof.

224. The preconcentrate according to claim 218, wherein the fatty acid oil mixture is derived from at least one oil chosen from marine oil, algae oil, plant-based oil, and microbial oil.

225. The preconcentrate according to claim 224, wherein the marine oil is a purified fish oil.

226. The preconcentrate according to claim 218, wherein the EPA:DHA weight ratio of the fatty acid oil mixture ranges from about 1:10 to 10:1, from about 1:8 to 8:1, from about 1:6 to 6:1, from about 1:5 to 5:1, from about 1:4 to 4:1, from about 1:3 to 3:1, from about 1:2 to 2:1, from about 1:1 to 2:1, or from about 1:2 to 1:3.

227. The preconcentrate according to claim 218, further comprising at least one additional oil chosen from medium-chain triglyceride, long-chain triglyceride and sesame oil.

228. The preconcentrate according to claim 218, further comprising at least one antioxidant.

229. The preconcentrate according to claim 218, wherein the preconcentrate is in the form of a gelatin capsule.

230. The preconcentrate according to claim 218, wherein the at least one surfactant is chosen from anionic surfactants chosen from salts of perfluorocarboxylic acids and perfluorosulphonic acid, alkyl sulphate salts, sulphate ethers, alkyl benzene sulphonate salts, and mixtures thereof; nonionic surfactants chosen from diacetyl monoglycerides, diethylene glycol monopalmitostearates, ethylene glycol monopalmitostearates, glyceryl behenates, glyceryl distearates, glyceryl monolinoleates, glyceryl mono-oleates, glyceryl monostearates, macrogol cetostearyl ethesr, macrogol 15 hydroxystearates, macrogol lauril ethers, macrogol monomethyl ethers, macrogol oleyl ethers, macrogol stearal, menfegol, mono and diglycerides, nonoxinols, octoxinols, polyoxamers, polyoxamer 188, polyoxamer 407, polyoxyl castor oils, polyoxyl hydrogenated castor oils, propylene glycol diacetates, propylene glycol laureates, propylene glycol monopalmitostearates, quillaia, sorbitan esters, sucrose esters, and mixtures thereof, and nonionic copolymers comprised of a central hydrophobic polymer of polyoxypropylene(poly(propylene oxide)) with a hydrophilic polymer of at least one of polyethylene(poly(ethylene oxide)), polyethylene ethers, sorbitan esters, polyoxyethylene fatty acid esters, polyethylated castor oil, and mixtures thereof; cationic surfactants chosen from quaternary ammonium compounds, cetylpyridinium chlorides, benzethonium chlorides, cetyl trimethylammonium bromides, and mixtures thereof; zwitterionic surfactants chosen from dodecyl betaines, coco amphoglycinates, cocamidopropyl betaines, and mixtures thereof; and mixtures thereof; a phospholipid, derivative thereof, analogue thereof, wherein the phospholipid or derivative or analogue thereof is chosen from phosphatidylcholines, phosphatidylethanolamines, phosphatidylglycerols, phosphatidylserines, phosphatidylinositols, and mixtures thereof, or any mixture thereof.

231. The preconcentrate according to claim 230, wherein the nonionic surfactants are chosen from polysorbate 20, polysorbate 40, polysorbate 60, polysorbate 80, and mixtures thereof.

232. The preconcentrate according to claim 218, wherein the ratio of fatty acid oil mixture:surfactant ranges from about 1:1 to about 10:1, from about 1:1 to about 8:1, from about 1:1 to about 6:1, from about 1:1 to about 4:1, or from about 1:1 to about 3:1.

233. The preconcentrate according to claim 218, wherein the at least one surfactant comprises from about 5% to about 55%, from about 10% to about 30%, or from about 10% to about 25%, by weight relative to the total weight of the preconcentrate.

234. The preconcentrate according to claim 233, wherein the at least one surfactant comprises about 20%, by weight relative to the total weight of the preconcentrate.

235. The preconcentrate according to claim 218, wherein the at least one statin or pharmaceutically acceptable salt, hydrate, solvate, or complex thereof is chosen from atorvastatin, cerivastatin, fluvastatin, itavastatin, lovastatin, mevastatin, rosuvastatin, simvastatin, pravastatin, pitavastatin, and pharmaceutically acceptable salts, hydrate, solvates, and complexes thereof.

236. The preconcentrate according to claim 235, wherein the at least one statin or pharmaceutically acceptable salt, hydrate, solvate, or complex thereof is chosen from simvastatin, atorvastatin, rosuvastatin, and pharmaceutically acceptable salts, hydrate, solvates, and complexes thereof.

237. The preconcentrate according to claim 236, wherein the at least one statin or pharmaceutically acceptable salt, hydrate, solvate, or complex thereof comprises atorvastatin or a calcium salt of atorvastatin.

238. The preconcentrate according to claim 218, wherein the at least one statin or pharmaceutically acceptable salt, hydrate, solvate, or complex thereof is present in an amount ranging from about 10 mg to about 80 mg.

239. The preconcentrate according to claim 218, wherein the at least one pharmaceutically-acceptable solvent is chosen from lower alcohols and polyols.

240. A pharmaceutical preconcentrate comprising:
a fatty acid oil mixture comprising from about 80% to about 88% eicosapentaenoic acid (EPA) and docosahexaenoic acid (DHA), by weight of the fatty acid oil mixture, wherein the EPA and DHA are in ethyl ester form;
at least one free fatty acid comprising from about 80% to about 88% eicosapentaenoic acid (EPA) and docosahexaenoic acid (DHA), by weight of the at least one free fatty acid, wherein the EPA and DHA are in free fatty acid form;
at least one surfactant chosen from polysorbate 20, polysorbate 80, and mixtures thereof; and
at least one statin chosen from atorvastatin, rosuvastatin, pravastatin, simvastatin, and a pharmaceutically acceptable salt, hydrate, solvate, or complex thereof.

241. A pharmaceutical preconcentrate comprising:
from about 45% to about 55% by weight, relative to the weight of the preconcentrate, of a fatty acid oil mixture comprising from about 80% to about 88% eicosapentaenoic acid (EPA) and docosahexaenoic acid (DHA) by weight of the fatty acid oil mixture, wherein the EPA and DHA are in a form chosen from ethyl ester and triglyceride;
from about 5% to about 15% of at least one free fatty acid, by weight relative to the weight of the preconcentrate;
from about 30% to about 40% of at least one surfactant, by weight relative to the weight of the preconcentrate; and
from about 0.5% to about 15% of at least one statin, or pharmaceutically acceptable salt, hydrate, solvate, or complex thereof by weight relative to the weight of the preconcentrate.

242. A pharmaceutical preconcentrate comprising:
from about 55% to about 65% by weight, relative to the weight of the preconcentrate, of a fatty acid oil mixture comprising from about 80% to about 88% eicosapentaenoic acid (EPA) and docosahexaenoic acid (DHA) by weight of the fatty acid oil mixture, wherein the EPA and DHA are in a form chosen from ethyl ester and triglyceride;
from about 5% to about 15% of at least one free fatty acid, by weight relative to the weight of the preconcentrate;
from about 20% to about 30% of at least one surfactant, by weight relative to the weight of the preconcentrate; and
from about 1% to about 10% of at least one statin or pharmaceutically acceptable salt, hydrate, solvate, or complex thereof, by weight relative to the weight of the preconcentrate.

243. The preconcentrate according to claim 241, wherein the at least one free fatty acid is chosen from oleic acid, ricioleic acid, linoleic acid, α-linolenic acid (ALA), gamma-linolenic acid (GLA) and erucic acid and the at least one surfactant is chosen from polysorbate 20 and polysorbate 80.

244. The preconcentrate according to claim 242, wherein the at least one free fatty acid is chosen from oleic acid, ricioleic acid, linoleic acid, α-linolenic acid (ALA), gamma-linolenic acid (GLA) and erucic acid and the at least one surfactant is chosen from polysorbate 20 and polysorbate 80.

245. A pharmaceutical preconcentrate comprising:
a fatty acid oil mixture comprising from about 80% to about 88% eicosapentaenoic acid (EPA) and docosahexaenoic acid (DHA) by weight of the fatty acid oil mixture, wherein the EPA and DHA are in ethyl ester form;
at least one free fatty acid comprising oleic acid;
at least one surfactant chosen from polysorbate 20 and polysorbate 80; and
at least one statin chosen from atorvastatin, rosuvastatin, simvastatin, and a pharmaceutically acceptable salt, hydrate, solvate, or complex thereof.

246. A self-nanoemulsifying drug delivery system (SNEDDS), self-microemulsifying drug delivery system (SMEDDS), or self-emulsifying drug delivery system (SEDDS) comprising a pharmaceutical preconcentrate comprising:
a fatty acid oil mixture comprising at least 75% eicosapentaenoic acid (EPA) and docosahexaenoic acid (DHA), by weight of the fatty acid oil mixture, wherein the EPA and DHA are in a form chosen from ethyl ester and triglyceride;
at least one free fatty acid;
at least one surfactant; and
at least one statin or pharmaceutically acceptable salt, hydrate, solvate, or complex thereof;
wherein the preconcentrate forms an emulsion in an aqueous solution.

247. The system according to claim 246, wherein the fatty acid oil mixture comprises at least 90% omega-3 fatty acids, by weight of the fatty acid oil mixture.

248. The system according to claim 246, wherein the fatty acid oil mixture further comprises at least one other fatty acid other than EPA and DHA in a form chosen from ethyl ester and triglyceride, wherein the at least one other fatty acid is chosen from α-linolenic acid (ALA), heneicosapentaenoic acid (HPA), docosapentaenoic acid (DPA), eicosatetraenoic acid (ETA), eicosatrienoic acid (ETE), stearidonic acid (STA), linoleic acid, α-linolenic acid (ALA), gamma-linolenic acid (GLA), arachidonic acid (AA), osbond acid, and mixtures thereof.

249. The system according to claim 246, wherein the at least one free fatty acid is chosen from EPA, DHA, ALA, HPA, DPA, ETA, ETE, STA, linoleic acid, GLA, AA, osbond acid, oleic acid, ricinoleic acid, erucic acid, and mixtures thereof.

250. The system according to claim 246, wherein the fatty acid oil mixture is derived from at least one oil chosen from marine oil, algae oil, plant-based oil, and microbial oil.

251. The system according to claim 250, wherein the marine oil is a purified fish oil.

252. The system according to claim 246, wherein the EPA:DHA weight ratio of the fatty acid oil mixture ranges from about 1:10 to 10:1, from about 1:8 to 8:1, from about 1:6 to 6:1, from about 1:5 to 5:1, from about 1:4 to 4:1, from about 1:3 to 3:1, from about 1:2 to 2:1, from about 1:1 to 2:1, or from about 1:2 to 1:3.

253. The system according to claim 246, further comprising at least one additional oil chosen from medium-chain triglyceride, long-chain triglyceride and sesame oil.

254. The system according to claim 246, wherein the preconcentrate further comprises at least one antioxidant.

255. The system according to claim 246, wherein the system is in the form of a gelatin capsule.

256. The system according to claim 246, wherein the at least one surfactant is chosen from anionic surfactants chosen from salts of perfluorocarboxylic acids and perfluorosulphonic acid, alkyl sulphate salts, sulphate ethers, alkyl benzene sulphonate salts, and mixtures thereof; nonionic surfactants chosen from diacetyl monoglycerides, diethylene glycol monopalmitostearates, ethylene glycol monopalmitostearates, glyceryl behenates, glyceryl distearates, glyceryl monolinoleates, glyceryl mono-oleates, glyceryl monostearates, macrogol cetostearyl ethesr, macrogol 15 hydroxystearates, macrogol lauril ethers, macrogol monomethyl ethers, macrogol oleyl ethers, macrogol stearas, menfegol, mono and diglycerides, nonoxinols, octoxinols, polyoxamers, polyoxamer 188, polyoxamer 407, polyoxyl castor oils, polyoxyl hydrogenated castor oils, propylene glycol diacetates, propylene glycol laureates, propylene glycol monopalmitostearates, quillaia, sorbitan esters, sucrose esters, and mixtures thereof, and nonionic copolymers comprised of a central hydrophobic polymer of polyoxypropylene(poly(propylene oxide)) with a hydrophilic polymer of at least one of polyethylene(poly(ethylene oxide)), polyethylene ethers, sorbitan esters, polyoxyethylene fatty acid esters, polyethylated castor oil, and mixtures thereof; cationic surfactants chosen from quaternary ammonium compounds, cetylpyridinium chlorides, benzethonium chlorides, cetyl trimethylammonium bromides, and mixtures thereof; zwitterionic surfactants chosen from dodecyl betaines, coco amphoglycinates, cocamidopropyl betaines, and mixtures thereof; and mixtures thereof; a phospholipid, derivative thereof, analogue thereof, wherein the phospholipid or derivative or analogue thereof is chosen from phosphatidylcholines, phosphatidylethanolamines, phosphatidylglycerols, phosphatidylserines, phosphatidylinositols, and mixtures thereof, or any mixture thereof.

257. The system according to claim 256, wherein the nonionic surfactants are chosen from polysorbate 20, polysorbate 40, polysorbate 60, polysorbate 80, and mixtures thereof.

258. The system according to claim 246, wherein the ratio of fatty acid oil mixture:surfactant ranges from about 1:1 to about 10:1, from about 1:1 to about 8:1, from about 1:1 to about 6:1, from about 1:1 to about 4:1, or from about 1:1 to about 3:1.

259. The system according to claim 246, wherein the at least one surfactant comprises from about 5% to about 55%, from about 10% to about 30%, or from about 10% to about 25%, by weight relative to the total weight of the system.

260. The system according to claim 259, wherein the at least one surfactant comprises about 20%, by weight relative to the total weight of the system.

261. The system according to claim 246, wherein the at least one statin or pharmaceutically acceptable salt, hydrate, solvate, or complex thereof is chosen from atorvastatin, cerivastatin, fluvastatin, itavastatin, lovastatin, mevastatin, rosuvastatin, simvastatin, pravastatin, pitavastatin, and pharmaceutically acceptable salts, hydrates, solvates, and complexes thereof.

262. The system according to claim 261, wherein the at least one statin or pharmaceutically acceptable salt, hydrate, solvate, or complex thereof is chosen from simvastatin, atorvastatin, rosuvastatin, and pharmaceutically acceptable salts, hydrates, solvates, and complexes thereof.

263. The system according to claim 262, wherein the at least one statin or pharmaceutically acceptable salt, hydrate, solvate, or complex thereof comprises atorvastatin or a calcium salt of atorvastatin.

264. The system according to claim 246, wherein the at least one statin or pharmaceutically acceptable salt, hydrate, solvate, or complex thereof is present in an amoung ranging from about 10 mg to about 80 mg.

265. The system according to claim 246, wherein the particle size of the emulsion ranges from about 150 nm to about 350 nm.

266. A method of treating at least one health problem in a subject in need thereof comprising administering to the subject a pharmaceutical composition comprising:
a pharmaceutically-effective amount of a fatty acid oil mixture comprising at least 75% eicosapentaenoic acid (EPA) and docosahexaenoic acid (DHA), by weight of the fatty acid oil mixture, wherein the EPA and DHA are in a form chosen from ethyl ester and triglyceride;
at least one free fatty acid; and
at least one statin or pharmaceutically acceptable salt, hydrate, solvate, or complex thereof;
wherein the at least one health problem is chosen from irregular plasma lipid levels, cardiovascular functions, immune functions, visual functions, insulin action, neuronal development, heart failure, and post myocardial infarction, mixed dyslipidemia, dyslipidemia, hypertriglyceridemia, and hypercholesterolemia.

267. The method according to claim 266, wherein said method treats elevated triglyceride levels, non-HDL cholesterol levels, LDL cholesterol levels and/or VLDL cholesterol levels.

268. The method according to claim 266, wherein the fatty acid oil mixture comprises at least 90% omega-3 fatty acids, by weight of the fatty acid oil mixture.

269. The method according to claim 266, wherein the fatty acid oil mixture further comprises at least one other fatty acid other than EPA and DHA in a form chosen from ethyl ester and triglyceride, wherein the at least one other fatty acid is chosen from α-linolenic acid (ALA), heneicosapentaenoic acid (HPA), docosapentaenoic acid (DPA), eicosatetraenoic acid (ETA), eicosatrienoic acid (ETE), stearidonic acid (STA), linoleic acid, α-linolenic acid (ALA), gamma-linolenic acid (GLA), arachidonic acid (AA), osbond acid, and mixtures thereof.

270. The method according to claim 266, wherein the at least one free fatty acid is chosen from EPA, DHA, ALA, HPA, DPA, ETA, ETE, STA, linoleic acid, GLA, AA, osbond acid, oleic acid, ricinoleic acid, erucic acid, and mixtures thereof.

271. The method according to claim 266, wherein the fatty acid oil mixture is derived from at least one oil chosen from marine oil, algae oil, plant-based oil, and microbial oil.

272. The method according to claim 271, wherein the marine oil is a purified fish oil.

273. The method according to claim 266, wherein the EPA:DHA weight ratio of the fatty acid oil mixture ranges from about 1:10 to 10:1, from about 1:8 to 8:1, from about 1:6 to 6:1, from about 1:5 to 5:1, from about 1:4 to 4:1, from about 1:3 to 3:1, from about 1:2 to 2:1, from about 1:1 to 2:1, or from about 1:2 to 1:3.

274. The method according to claim 266, further comprising at least one additional oil chosen from medium-chain triglyceride, long-chain triglyceride and sesame oil.

275. The method according to claim 266, wherein the composition further comprises at least one antioxidant.

276. The method according to claim 266, wherein the composition further comprises at least one superdisintegrant chosen from crosscarmelose, crospovidone, and sodium starch glycolate, and wherein the composition is loaded into a tablet.

277. The method according to claim 276, wherein the at least one superdisintegrant comprises from about 1% to about 20%, by weight relative to the total weight of the pharmaceutical composition.

278. The method according to claim 266, wherein the composition is in the form of a gelatin capsule.

279. The method according to claim 266, wherein the composition is administered once, twice, or three times per day.

280. The method according to claim 266, wherein the composition further comprises at least one surfactant chosen from anionic surfactants chosen from salts of perfluorocarboxylic acids and perfluorosulphonic acid, alkyl sulphate salts, sulphate ethers, alkyl benzene sulphonate salts, and mixtures thereof; nonionic surfactants chosen from diacetyl monoglycerides, diethylene glycol monopalmitostearates, ethylene glycol monopalmitostearates, glyceryl behenates, glyceryl distearates, glyceryl monolinoleates, glyceryl mono-oleates, glyceryl monostearates, macrogol cetostearyl ethesr, macrogol 15 hydroxystearates, macrogol lauril ethers, macrogol monomethyl ethers, macrogol oleyl ethers, macrogol stearas, menfegol, mono and diglycerides, nonoxinols, octoxinols, polyoxamers, polyoxamer 188, polyoxamer 407, polyoxyl castor oils, polyoxyl hydrogenated castor oils, propylene glycol diacetates, propylene glycol laureates, propylene glycol monopalmitostearates, quillaia, sorbitan esters, sucrose esters, and mixtures thereof, and nonionic copolymers comprised of a central hydrophobic polymer of polyoxypropylene(poly(propylene oxide)) with a hydrophilic polymer of at least one of polyethylene(poly(ethylene oxide)), polyethylene ethers, sorbitan esters, polyoxyethylene fatty acid esters, polyethylated castor oil, and mixtures thereof; cationic surfactants chosen from quaternary ammonium compounds, cetylpyridinium chlorides, benzethonium chlorides, cetyl trimethylammonium bromides, and mixtures thereof; zwitterionic surfactants chosen from dodecyl betaines, coco amphoglycinates, cocamidopropyl betaines, and mixtures thereof; and mixtures thereof, a phospholipid, derivative thereof, analogue thereof, wherein the phospholipid or derivative or analogue thereof is chosen from phosphatidylcholines, phosphatidylethanolamines, phosphatidylglycerols, phosphatidylserines, phosphatidylinositols, and mixtures thereof, or any mixture thereof; to form a pharmaceutical preconcentrate.

281. The method according to claim 280, wherein the nonionic surfactants are chosen from polysorbate 20, polysorbate 40, polysorbate 60, polysorbate 80, and mixtures thereof.

282. The method according to claim 280, wherein the preconcentrate forms a self-nanoemulsifying drug delivery system (SNEDDS), self-microemulsifying drug delivery system (SMEDDS), or self-emulsifying drug delivery system (SEDDS) in an aqueous solution.

283. The method according to claim 266, wherein the at least one statin or pharmaceutically acceptable salt, hydrate, solvate, or complex thereof is chosen from atorvastatin, cerivastatin, fluvastatin, itavastatin, lovastatin, mevastatin, rosuvastatin, simvastatin, pravastatin, pitavastatin, and pharmaceutically acceptable salts, hydrates, solvates, and complexes thereof.

284. The method according to claim 283, wherein the at least one statin or pharmaceutically acceptable salt, hydrate, solvate, or complex thereof is chosen from simvastatin, atorvastatin, rosuvastatin, and pharmaceutically acceptable salts, hydrates, solvates, and complexes thereof.

285. The method according to claim 284, wherein the at least one statin or pharmaceutically acceptable salt, hydrate, solvate, or complex thereof comprises atorvastatin or a calcium salt of atorvastatin.

286. A method for enhancing at least one parameter chosen from hydrolysis, solubility, bioavailability, absorption, and combinations thereof of eicosapentaenoic acid (EPA) and docosahexaenoic acid (DHA) comprising combining:
a fatty acid oil mixture comprising at least 75% eicosapentaenoic acid (EPA) and docosahexaenoic acid (DHA), by weight of the fatty acid oil mixture, wherein the EPA and DHA in a form chosen from ethyl ester and triglyceride;
at least one free fatty acid; and
at least one statin or pharmaceutically acceptable salt, hydrate, solvate, or complex thereof.

287. A method for enhancing at least one parameter chosen from hydrolysis, solubility, bioavailability, absorption, and combinations thereof of eicosapentaenoic acid (EPA) and docosahexaenoic acid (DHA) comprising combining:
a fatty acid oil mixture comprising at least 75% eicosapentaenoic acid (EPA) and docosahexaenoic acid (DHA), by weight of the fatty acid oil mixture, wherein the EPA and DHA in a form chosen from ethyl ester and triglyceride;
at least one free fatty acid;
at least one surfactant; and
at least one statin or pharmaceutically acceptable salt, hydrate, solvate, or complex thereof thereof;
wherein the fatty acid oil mixture, the at least one free fatty acid, the at least one surfactant, and the at least one statin or pharmaceutically acceptable salt, hydrate, solvate, or complex thereof form a preconcentrate.

288. The method according to claim 287, wherein the at least one surfactant is chosen from anionic surfactants chosen from salts of perfluorocarboxylic acids and perfluorosulphonic acid, alkyl sulphate salts, sulphate ethers, alkyl benzene sulphonate salts, and mixtures thereof; nonionic surfactants chosen from diacetyl monoglycerides, diethylene glycol monopalmitostearates, ethylene glycol monopalmitostearates, glyceryl behenates, glyceryl distearates, glyceryl monolinoleates, glyceryl mono-oleates, glyceryl monostearates, macrogol cetostearyl ethesr, macrogol 15 hydroxystearates, macrogol lauril ethers, macrogol monomethyl ethers, macrogol oleyl ethers, macrogol stearas, menfegol, mono and diglycerides, nonoxinols, octoxinols, polyoxamers, polyoxamer 188, polyoxamer 407, polyoxyl castor oils, polyoxyl hydrogenated castor oils, propylene glycol diacetates, propylene glycol laureates, propylene glycol monopalmitostearates, quillaia, sorbitan esters, sucrose esters, and mixtures thereof, and nonionic copolymers comprised of a central hydrophobic polymer of polyoxypropylene(poly(propylene oxide)) with a hydrophilic polymer of at least one of polyethylene(poly(ethylene oxide)), polyethylene ethers, sorbitan esters, polyoxyethylene fatty acid esters, polyethylated castor oil, and mixtures thereof; cationic surfactants chosen from quaternary ammonium compounds, cetylpyridinium chlorides, benzethonium chlorides, cetyl trimethylammonium bromides, and mixtures thereof; zwitterionic surfactants chosen from dodecyl betaines, coca amphoglycinates, cocamidopropyl betaines, and mixtures thereof; and mixtures thereof; a phospholipid, derivative thereof, analogue thereof, wherein the phospholipid or derivative or analogue thereof is chosen from phosphatidylcholines, phosphatidylethanolamines, phosphatidylglycerols, phosphatidylserines, phosphatidylinositols, and mixtures thereof, or any mixture thereof.

289. The method according to claim 288, wherein the nonionic surfactants are chosen from polysorbate 20, polysorbate 40, polysorbate 60, polysorbate 80, and mixtures thereof.

290. The method according to claim 287, wherein the preconcentrate comprises:
a fatty acid oil mixture comprising from about 80% to about 88% eicosapentaenoic acid (EPA) and docosahexaenoic acid (DHA) by weight of the fatty acid oil mixture, wherein the EPA and DHA are in ethyl ester form;
at least one free fatty acid comprising oleic acid;
at least one surfactant chosen from polysorbate 20 and polysorbate 80; and
at least one statin chosen from pravastatin, atorvastatin, rosuvastatin, simvastatin, and a pharmaceutically acceptable salt, hydrate, solvate, or complex thereof.

291. The method according to claim 287, wherein the preconcentrate comprises:
a fatty acid oil mixture comprising from about 80% to about 8 eicosapentaenoic acid (EPA) and docosahexaenoic acid (DHA) by weight of the fatty acid oil mixture, wherein the EPA and DHA are in ethyl ester form;
at least one free fatty acid comprising linoleic acid;
at least one surfactant chosen from polysorbate 20 and polysorbate 80; and
at least one statin chosen from pravastatin, atorvastatin, rosuvastatin, simvastatin, and a pharmaceutically acceptable salt, hydrate, solvate, or complex thereof.

292. The method according to claim 287, wherein the preconcentrate comprises:
a fatty acid oil mixture comprising from about 80% to about 88% eicosapentaenoic acid (EPA) and docosahexaenoic acid (DHA), by weight of the fatty acid oil mixture, wherein the EPA and DHA are in ethyl ester form;
at least one free fatty acid comprising from about 80% to about 88% eicosapentaenoic acid (EPA) and docosahexaenoic acid (DHA), by weight of the at least one free fatty acid, wherein the EPA and DHA are in free fatty acid form;
at least one surfactant chosen from polysorbate 20, polysorbate 80, and mixtures thereof;
and at least one statin chosen from pravastatin, atorvastatin, rosuvastatin, simvastatin, and a pharmaceutically acceptable salt, hydrate, solvate, or complex thereof.

293. The method according to claim 287, wherein the preconcentrate
forms a self-nanoemulsifying drug delivery system (SNEDDS), self-microemulsifying drug delivery system (SMEDDS), or self-emulsifying drug delivery system (SEDDS) in an aqueous solution.

294. The method according to claim 293, wherein the system comprises an emulsion with a particle size ranging from about 150 nm to about 350 nm.

295. A pharmaceutical composition comprising a fatty acid oil mixture comprising at least 75% eicosapentaenoic acid (EPA) and docosahexaenoic acid (DHA), by weight of the fatty acid oil mixture, wherein the EPA and DHA are in a form chosen from ethyl ester and triglyceride; at least one free fatty acid; and at least one statin or pharmaceutically acceptable salt, hydrate, solvate, or complex thereof for the treatment of at least one health problem chosen from post myocardial infarction, mixed dyslipidemia, dyslipidemia, hypertriglyceridemia, and hypercholesterolemia.

296. A pharmaceutical preconcentrate comprising a fatty acid oil mixture comprising at least 75% eicosapentaenoic acid (EPA) and docosahexaenoic acid (DHA), by weight of the fatty acid oil mixture, wherein the EPA and DHA are in a form chosen from ethyl ester and triglyceride; at least one free fatty acid; at least one surfactant; and at least one statin or pharmaceutically acceptable salt, hydrate, solvate, or complex thereof for the treatment of at least one health problem chosen from post myocardial infarction, mixed dyslipidemia, dyslipidemia, hypertriglyceridemia, and hypercholesterolemia.

297. A self-nanoemulsifying drug delivery system (SNEDDS), self-microemulsifying drug delivery system (SMEDDS), or self-emulsifying drug delivery system (SEDDS) comprising a pharmaceutical preconcentrate comprising: a fatty acid oil mixture comprising at least 75% eicosapentaenoic acid (EPA) and docosahexaenoic acid (DHA), by weight of the fatty acid oil mixture, wherein the EPA and DHA are in a form chosen from ethyl ester and triglyceride; at least one free fatty acid; at least one surfactant; and at least one statin or pharmaceutically acceptable salt, hydrate, solvate, or complex thereof; wherein the preconcentrate forms an emulsion in an aqueous solution for the treatment of at least one health problem chosen from post myocardial infarction, mixed dyslipidemia, dyslipidemia, hypertriglyceridemia, and hypercholesterolemia.

298. The composition according to claim 217, wherein the capsule comprises two compartments, a first compartment comprising the fatty acid oil mixture and a second compartment comprising the at least one statin or pharmaceutically acceptable salt, hydrate, solvate, or complex thereof.

299. The composition according to claim 298, wherein the first compartment comprising the fatty acid oil mixture further comprises the at least one free fatty acid.

300. The preconcentrate according to claim 229, wherein the capsule comprises two compartments, a first compartment comprising the fatty acid oil mixture and a second compartment comprising the at least one statin or pharmaceutically acceptable salt, hydrate, solvate, or complex thereof.

301. The preconcentrate according to claim 300, wherein the first compartment comprising the fatty acid oil mixture further comprises the at least one free fatty acid and the at least one surfactant.

302. The composition according to claim 217, wherein the at least one statin or pharmaceutically acceptable salt, hydrate, solvate, or complex thereof comprises microcapsules suspended in the fatty acid oil mixture

303. The composition according to claim 302, wherein the microcapsules are encapsulated in a material chosen from cyclodextrin and alginate.

304. The preconcentrate according to claim 229, wherein the at least one statin or pharmaceutically acceptable salt, hydrate, solvate, or complex thereof comprises microcapsules suspended in the fatty acid oil mixture.

305. The preconcentrate according to claim 304, wherein the microcapsules are encapsulated in a material chosen from cyclodextrin and alginate.

306. The composition according to claim 217, wherein the gelatin capsule comprises a gelatin shell comprising the at least one statin or pharmaceutically acceptable salt, hydrate, solvate, or complex thereof.

307. The composition according to claim 217, wherein the at least one statin or pharmaceutically acceptable salt, hydrate, solvate, or complex thereof forms an outer layer on the gelatin capsule.

308. The preconcentrate according to claim 229, wherein the gelatin capsule comprises a gelatin shell comprising the at least one statin or pharmaceutically acceptable salt, hydrate, solvate, or complex thereof.

309. The preconcentrate according to claim 229, wherein the at least one statin or pharmaceutically acceptable salt, hydrate, solvate, or complex thereof forms an outer layer on the gelatin capsule.

310. A pharmaceutical preconcentrate comprising:
from about 45% to about 55% by weight, relative to the weight of the preconcentrate, of a fatty acid oil mixture comprising from about 80% to about 88% eicosapentaenoic acid (EPA) and docosahexaenoic acid (DHA) by weight of the fatty acid oil mixture, wherein the EPA and DHA are in ethyl ester form;
polysorbate 20;
oleic acid:
antioxidant; and
at least one statin or pharmaceutically acceptable salt, hydrate, solvate or complex thereof.

311. A pharmaceutical preconcentrate comprising:
from about 45% to about 55% by weight, relative to the weight of the preconcentrate, of a fatty acid oil mixture comprising from about 80% to about 88% eicosapentaenoic acid (EPA) and docosahexaenoic acid (DHA) by weight of the fatty acid oil mixture, wherein the EPA and DHA are in ethyl ester form;
from about 30% to about 40% of polysorbate 20, by weight relative to the weight of the preconcentrate;
from about 5% to about 15% of oleic acid, by weight relative to the weight of the e preconcentrate;
antioxidant; and
from about 0.5 to about 15% of at least one statin or pharmaceutically acceptable salt, hydrate, solvate or complex thereof by weight relative to the weight of the preconcentrate.

312. A pharmaceutical preconcentrate comprising:
a fatty acid oil mixture comprising from about 45% to about 55% by weight, relative to the weight of the preconcentrate, of a fatty acid oil mixture comprising at least 95% of eicosapentaenoic acid (EPA) by weight of the fatty acid oil mixture, wherein the EPA is in ethyl ester form;
polysorbate 20;
oleic acid;
antioxidant; and
at least one statin or pharmaceutically acceptable salt, hydrate, solvate or complex thereof.

313. A pharmaceutical preconcentrate comprising:
a fatty acid oil mixture comprising from about 45% to about 55% by weight, relative to the weight of the preconcentrate, of a fatty acid oil mixture comprising at least 95% of docosahexaenoic acid (DHA) by weight of the fatty acid oil mixture, wherein the DHA is in ethyl ester form;
polysorbate 20;
oleic acid;
antioxidant; and
at least one statin or pharmaceutically acceptable salt, hydrate, solvate or complex thereof.

314. A self-nanoemulsifying drug delivery system (SNEDDS), self-microemulsifying drug delivery system (SMEDDS), or self-emulsifying drug delivery system (SEDDS) comprising a pharmaceutical preconcentrate comprising:
a fatty acid oil mixture comprising at least 75% eicosapentaenoic acid (EPA) and docosahexaenoic acid (DHA), by weight of the fatty acid oil mixture, wherein the EPA and DHA are in a form chosen from ethyl ester and triglyceride;
polysorbate 20;
oleic acid;
antioxidant; and
at least one statin or pharmaceutically acceptable salt, hydrate, solvate, or complex thereof;
wherein the preconcentrate forms an emulsion in an aqueous solution.
